(19) United States
(12) Patent Application Publication
(10) Pub. No.: US 2006/0045884 A1
(43) Pub. Date: Mar. 2, 2006

(54) Vaccines for autoimmune and infectious disease

(75) Inventor: Jeffrey Molldrem, Houston, TX (US)

Correspondence Address: Steven L. Highlander;FULBRIGHT & JAWORSKI L.L.P., Suite 2400, 600 Congress Avenue, Austin, TX, 78701-3271, US

(73) Assignees: Board of Regents; The University of Texas System

(21) Appl. No.: 10/927,435

(22) Filed: Aug. 26, 2004

(51) Int. Cl. A61K 39/00

(52) U.S. Cl. 424/185.1

(57) ABSTRACT

The present invention provides HLA-restricted antigens as vaccines for treating or preventing autoimmune diseases or conditions, translpant rejection or vasculitis in a patient. In particular aspects, there is provided Pr3, a myeloid tissue-restricted protein and a HLA-A2.1-restricted self-peptide, PR1, derived from Pr3, which can be used to elicit PR1-specific CTLs. The present invention also provides a HLA-restricted-peptide myeloperoxidases-based methods.

This application claims benefit of priority to U.S. Provisional Application Ser. No. 60/489,238, filed Aug. 26, 2003, the entire contents of which are hereby incorporated by reference.

The government owns rights in the present invention pursuant to grant numbers RO1 CA81247-02 and RO1 CA49639-11 from the National Institutes of Health.

### BACKGROUND OF THE INVENTION

1. Field of the Invention

The present invention relates generally to the fields of cancer and immunotherapy. More particularly, it concerns the identification of immunotherapeutic peptides and the development of peptide vaccines for the treatment and prevention of autoimmune diseases, transplant rejection, post-transplant antitumor immunity and vasculitis.

2. Description of Related Art

The immune system has long been implicated in the control of cancer, however, evidence for specific and efficacious immune responses in human cancer have been lacking. In chronic myelogenous leukemia (CML), either allogeneic bone marrow transplant (BMT) or interferon-α2b (IFN-α2b) therapy have resulted in complete remission, but the mechanism for disease control is unknown and may involve immune antileukemic responses.

Based on evidence in the art, it is thought that lymphocytes play a role in meditating an antileukemia effect. Studies have demonstrated that allogeneic donor lymphocyte infusions (DLI) have been used to treat relapse of myeloid leukemia after allogeneic BMT (Giralt and Kolb, 1996; Kolb and Holler, 1997; Kolb et al., 1995; Kolb et al., 1996; Antin, 1993). Lymphocyte transfusion from the original bone marrow (BM) donor induces both hematological and cytogenetic responses in approximately 70% to 80% of patients with chronic myelocytic leukemia (CML) in chronic phase (CP) (Kolb et al., 1996, Holler, 1997). Remissions after DLI for AML are generally not as durable as those obtained in chronic phase CML, which may reflect the rapid kinetics of tumor growth outpacing the kinetics of the developing immune response. Additionally, most patients with myeloid forms of leukemia will die from the disease unless they can be treated with allogeneic bone marrow transplant, where the associated graft versus leukemia (GVL) effect cures patients. However, graft-versus-host disease (GVHD) and transplant-related toxicity limit this treatment. It is believed that GVL may be separable from GHVD, and that targeting the immune response toward leukemia-associated antigens will allow for the transfer of GVL to patients without GVHD.

Thus, if more antigens (i.e., leukemia antigens or antigens aganist other cancers) could be determined, and if large numbers of the most potent antigen-specific cytotoxic T lymphocytes (CTLs) could be obtained, it would allow for development of leukemia-specific therapies, breast cancer specific therapies, etc. using the antigens as a targets for vaccines or for generating specific T cells for use in adoptive immunotherapy.

Various methods have been used in the art to determine the nature of the target antigens, such as leukemia-specific antigens, of the GVL effect. For instance, tissue-restricted minor histocompatibility antigens (mHA) that are derived from proteins expressed only in recipient hematopoietic tissue have been shown to be the targets of alloreactive donor T cells. Heterologous T cell clones that demonstrate alloreactivity toward different mHA have been established from patients with severe GVHD following BMT with an HLA-matched donor (Faber et al., 1995a; Faber et al., 1995b; Faber et al., 1996; van der Harst et al., 1994; Molldrem et al., 2000; Gao et al., 2000; den Haan et al., 1998; Clark et al., 2001). Some of these mHA-specific CTL clones react only with hematopoietic-derived cells, suggesting tissue specificity (Faber et al., 1996; den Haan et al., 1998), and therefore potentially shared antigens on leukemia. Expression of two human mHAs, identified as HA-1 and HA-2, is confined to hematopoietic tissues. HA-2 was identified as a peptide derived from the non-filament-forming class I myosin family by using mHA-reactive CTL clones to screen peptide fractions eluted from MHC class I molecules (den Haan et al., 1995; Faber et al., 1995a).

Thus, while various methodologies has successfully defined some CTL alloantigens, it is extremely labor intensive and it is unclear whether CTL specific for any minor antigens identified thus far convey leukemia-specific immunity without concomitant GVHD. In one study, GVHD correlated closely with differences in the minor antigen HA-I in HLA identical sibling transplants (Goulmy et al., 1996; Dolstra et al., 1997). Furthermore, a practical limit of any immunotherapy approach targeting these mHAs is that only 10% of individuals would be expected to have the relevant HA-1 alternate allele, and <1% would have the HA-2 alternate allele, which makes donor availability quite limiting.

An alternative immunological or deductive method to determine leukemia-specific CTL epitopes has been applied to determine whether BCR-ABL fusion region peptides could elicit CML-specific T cell responses. Peptides are synthesized based upon an “educated guess” of which proteins are potential target antigens for a selective anti-leukemia CTL response. The proteins are then examined for short peptides that fit the binding motif of the most common HLA alleles based on the amino acid sequence. Using this method, these peptides are then synthesized, tested for binding to the HLA allele, and used to elicit peptide-specific CTL responses in vitro. Since BCR-ABL is present in nearly all (92%) Philadelphia chromosome-positive CML patients, it is thought to represent a potentially unique leukemia antigen. The ABL coding sequences upstream (5′) of exon II on chromosome 9 are translocated to chromosome 22 and fused inframe with the BCR gene downstream (3′) of exon III, resulting in a chimeric mRNA (b3a2) (the most common transcript) which is translated into a chimeric protein (p210BCR-ABL). Translation of b3a2 mRNA results in the coding of a unique amino acid (lysine) within the fusion region. Some HLA-B8-restricted overlapping peptides inclusive of this lysine could bind to HLA-B8 and could be used to elicit T cell proliferative responses when the peptide was either pulsed onto HLA-matched normal antigen presenting cells or onto HLA-B8 positive CML cells (Dermime et al., 1995; Bocchia et al., 1995; Bocchia et al., 1996; Faber et al., 1995b; Faber et al., 1996; van der Harst et al., 1994). However, when the b3a2 peptides were used to elicit b3a2-specific T lymphocyte lines in vitro, the resulting T cells could not specifically lyse fresh CML cells which had not previously been pulsed with the peptide (Bocchia et al., 1996; van der Harst et al., 1994). This could be due to a low affinity of the peptide-specific CTL or the peptide may not be processed or presented on CML cells. More recently, b3a2-specific CTL were identified in the peripheral blood of CML patients using soluble b3a2 peptide/MHC tetramers (Clark et al., 2001). Although the tetramer-positive CTL from the patients were not tested to determine whether they could kill CML target cells, b3a2-specific CTL elicited in vitro from healthy donors were able to kill CML cells (den Haan et al., 1995). This suggests that bcr-abl fusion peptides may be targets of GVL reactions. Although peptides derived from the b3a2 fusion region given as a vaccine to CML patients elicited CTL immunity in 3 of 12 patients, no clinical benefit was noted (Pinilla-lbarz et al., 2000).

In tumors other than leukemia, such as melanoma and breast cancer, many peptide antigens have been identified as targets of tumor-specific CTL. What is clear from these studies is that nearly all of the tumor antigens identified are derived from normal tissue proteins (Nanda and Sercarz, 1995; Boon et al., 1997). It is now accepted that many self-antigenic determinants have not induced self-tolerance and that these peptide determinants supply target structures for autoimmune attack (Nanda and Sercarz, 1995; Rosenberg and White, 1996; Goulmy et al., 1996; Dermime et al., 1995). Since these proteins are often aberrantly expressed or overexpressed in the tumor, there is relative tumor specificity by CTL that recognize these epitopes (Pardoll, 2002; Pardoll, 1994; Bocchia et al., 1996; Pinilla-Ibarz et al., 2000). Similarly in leukemia, CTL immunity to the Wilm's tumor antigen WT-1, which is aberrantly expressed in various forms of leukemia, has been demonstrated to kill CML CD34+ progenitor cells (Gao et al., 2000).

Melanoma peptide antigens that are derived from MAGE-3 proteins, for example, are presented to melanoma-specific CTLs by HLA-A1 and HLA-A2 (Nanda and Sercarz, 1995; Boon et al., 1997; Rosenberg and White, 1996). This protein belongs to a family of proteins which are expressed in melanoma cells and in normal testis. A MAGE-3 derived peptide was determined to be immunogenic by separate groups using different techniques, one using an immunological method (Pardoll, 2002) and the other a genetic method that uses tumor antigen-deficient mutants (Nanda and Sercarz, 1995). Recently, a phase I clinical trial using MAGE-3 to vaccinate melanoma patients resulted in some clinical responses (Pardoll, 1994). In addition, tyrosinase, gp100, and Melan-A-MART-1 are also normal self-proteins specific to the melanocyte lineage and T-cells specific for determinants on each of these antigens can be found in a large majority of melanoma patients (Sturrock et al., 1992; Chen et al., 1994). Two recent phase II vaccine trials demonstrated clinical efficacy of active immunotherapy using these target antigens as a peptide vaccine or as a antigen-pulsed dendritic cell vaccine.

PR1, an HLAA2.1-restricted nonamer derived from proteinase 3 (P3), was identified as a leukemia-associated antigen (Molldrem et al., 2000; Molldrem et al., 1996; Molldrem et al., 1997; Molldrem et al., 1999; Molldrem et al., 2003 each incorporated herein by reference in their entirety). The finding that PR1 is a leukemia-associated antigen has been independently confirmed by Burchert et al. (2002) and Scheibenbogen et al. (2002). These studies have thus established PR1 as a human leukemia-associated antigen and have established that PR1-specific CTL contribute to the elimination of CML.

Although some tumor specific antigens have been identified as putative immunotherapeutic targets, there still is a great need in the art to identify more antigens and develop immunotherapeutic methods that target different cancers. New approaches for treatment of disease/conditions such as autoimmune diseases, organ transplant rejection and vasculitis are therefore needed.

### SUMMARY OF THE INVENTION

In accordance with the present invention, there is provided a pmethod of treating an autoimmune disease in a patient comprising administering to said patient a therapeutically effective amount of a vaccine comprising a HLA-A2-restricted peptide. The HLA-A2-restricted peptide may be a proteinase-3 peptide, a neutrophil elastase peptide, a myeloperoxidase peptide, a cyclin E1 peptide, cyclin D, or a cyclin E2 peptide.

The cyclin D may comprise the sequence LLGATCMFV (SEQ ID NO:61) or a fragment thereof, the cyclin E1 peptide may comprise the sequence ILLDWLMEV (SEQ ID NO:62), the cyclin E2 peptide may comprise the sequence ILLDWLLEV (SEQ ID NO:63) or a fragment thereof, or the myleoperoxidase peptide may comprise the sequence of MY2 (SEQ ID NO:32) or MY4 (SEQ ID NO:30) or a fragment thereof.

The vaccine may further comprise a disease-associated HLA-A3 restricted peptide, a disease-associated HLA-A11 restricted peptide, a disease-associated HLA-B7 restricted peptide, a disease-associated HLA-B27 restricted peptide, and/or a disease-associated HLA-B35 restricted peptide.

The proteinase-3 peptide may be selected from the group consisting of VLQELNVTV (SEQ ID NO:1), RFLPDFFTRV (SEQ ID NO:3), VLQELNVTVV (SEQ ID NO:4), NLSASVTSV (SEQ ID NO:5), IIQGIDSFV (SEQ ID NO:6), VLLALLLISGA (SEQ ID NO:7), QLPQQDQPV (SEQ ID NO:10) and FLNNYDAENKL (SEQ ID NO: 11) or a fragment thereof. Alternatively, the proteinase-3 peptide may be a modified peptide selected from the group consisting of VLQELWTV (SEQ ID NO:26), VLQELNVKV (SEQ ID NO:27), VLQELWKV (SEQ ID NO:28) and VMQELWTV (SEQ ID NO:29) or a fragment thereof.

The autoimmune disease may be vasculitis, Wegener's granulomatosis, Addison's disease, alopecia, ankylosing spondylitis, antiphospholipid syndrome, Behcet's disease, celiac disease, chronic fatigue syndrome, Crohn's disease, ulcerative colitis, type I diabetes, fibromyalgia, autoimmune gastritis, Goodpasture syndrome, Graves' disease, idiopathic thrombocytopenic purpura (ITP), lupus, Meniere's multiple sclerosis, myasthenia gravis, pemphigus vulgaris, primary biliary cirrhosis, psoriasis, rheumatoid arthritis, rheumatic fever, sarcoidosis, scleroderma, vitiligo, vasculitis, small vessel vasculitis, hepatitis, primary biliary cirrhosis, rheumatoid arthritis, Chrohn's disease, ulcerative colitis, sarcoidosis, scleroderma, graft versus host disease (acute and chronic), aplastic anemia, or cyclic neutropenia.

In another embodiment, there is provided a method of treating or preventing an infectious disease in a patient comprising administering to said patient a therapeutically effective amount of a vaccine comprising a HLA-A2-restricted peptide, wherein the HLA-A2-restricted peptide is a proteinase-3 peptide, a neutrophil elastase peptide, a myeloperoxidase peptide, a cyclin E1 peptide, cyclin D, or a cyclin E2 peptide. The infectious disease may be a viral hepatitis, such as hepatitis B or hepatitis C.

The cyclin D may comprise the sequence LLGATCMFV (SEQ ID NO:61) or a fragment thereof, the cyclin E1 peptide may comprise the sequence ILLDWLMEV (SEQ ID NO:62), the cyclin E2 peptide may comprise the sequence ILLDWLLEV (SEQ ID NO:63) or a fragment thereof, or the myleoperoxidase peptide may comprise the sequence of MY2 (SEQ ID NO:32) or MY4 (SEQ ID NO:30) or a fragment thereof.

The vaccine may further comprise a disease-associated HLA-A3 restricted peptide, a disease-associated HLA-Al restricted peptide, a disease-associated HLA-B7 restricted peptide, a disease-associated HLA-B27 restricted peptide, and/or a disease-associated HLA-B35 restricted peptide.

The proteinase-3 peptide may be selected from the group consisting of VLQELNVTV (SEQ ID NO:1), RFLPDFFTRV (SEQ ID NO:3), VLQELNVTVV (SEQ ID NO:4), NLSASVTSV (SEQ ID NO:5), IIQGIDSFV (SEQ ID NO:6), VLLALLLISGA (SEQ ID NO:7), QLPQQDQPV (SEQ ID NO:10) and FLNNYDAENKL (SEQ ID NO:11) or a fragment thereof. Alternatively, the proteinase-3 peptide may be a modified peptide selected from the group consisting of VLQELWTV (SEQ ID NO:26), VLQELNVKV (SEQ ID NO:27), VLQELWKV (SEQ ID NO:28) and VMQELWTV (SEQ ID NO:29) or a fragment thereof.

In yet another embodiment, there is provided a method of generating HLA-restricted peptide specific cytotoxic T-lymphocytes (CTLs) comprising (a) obtaining a target peptide pulsed onto antigen-presenting cells to expand the CTL ex vivo; (b) concentrating high affinity HLA-restricted peptide specific CTLs; and (c) expanding the concentrated HLA-restricted peptide specific CTLs. The target peptide may be provided in a limited amount such as at least 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 15, 20, 25, 30, 35, 40, 45, 50 60, 70, 80, 90, 100, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, or more [M.

The method may further comprise purifying the concentrated HLA-restricted peptide specific CTLs. Step (b) may occur prior to, after, or both before and after purifying the concentrated HLA-restricted peptide-specific CTLs. Purifying may comprise high-speed flow cytometry. The target peptide may depend upon the donor and the peptide epitope. Concentrating high affinity HLA-restricted peptide specific CTLs comprises using microbeads and/or high speed sorting. The method may further comprise concentrating low affinity HLA-restricted peptide specific CTLs, and may even further comprise expanding the concentrated low affinity HLA-restricted peptide specific CTLs. The method may also further comprising adoptive T cell immunotherapy, and may also further comprise expansion of adoptively transferred peptide-specific T lymphocytes, and may also further comprise vaccinating the recipient with the specific peptide combined with an adjuvant after the transfer of the peptide-specific T lymphocytes.

In still yet another embodiment, there is provided method for treating or preventing an autoimmune or infectious disease in a patient comprising (a) contacting CTLs of said patient with a HLA-A2-restricted peptide, wherein the HLA-A2-restricted peptide is a proteinase 3 peptide, a neutrophil elastase peptide, a myeloperoxidase peptide, a cyclin E1 peptide, cyclin D, or a cyclin E2 peptide; and (b) administering a therapeutically effective amount of said CTLs to the patient. The method may further comprise expanding said CTL's by ex vivo or in vivo methods prior to administration. Contacting may comprise providing an antigen presenting cell containing said peptide. The method may further comprise providing CTLs transfected with a T cell receptor specific for the peptide/HLA-A2 ligand.

The autoimmune disease may be vasculitis, Wegner's granulomatoses Addison's disease, alopecia, ankylosing spondylitis, antiphospholipid syndrome, Behcet's disease, celiac disease, chronic fatigue syndrome, Crohn's disease, ulcerative colitis, type I diabetes, fibromyalgia, autoimmune gastritis, Goodpasture syndrome, Graves' disease, idiopathic thrombocytopenic purpura (ITP), lupus, Meniere's multiple sclerosis, myasthenia gravis, pemphigus vulgaris, primary biliary cirrhosis, psoriasis, rheumatoid arthritis, rheumatic fever, sarcoidosis, scleroderma, vitiligo, vasculitis, small vessel vasculitis, hepatitis, primary biliary cirrhosis, rheumatoid arthritis, Chrohn's disease, ulcerative colitis, sarcoidosis, scleroderma, graft versus host disease (acute and chronic), aplastic anemia, myelodysplastic syndrome, cyclic neutropenia, T-LGL (T-large granular lymphocytic) leukemia. The infectious disease may be a viral hepatitis, such as hepatitis B or hepatitis C. The therapeutically effective amount of CTL cells required to provide therapeutic benefit is from about 0.1×10⁵ to about 5×10⁷ cells per kilogram weight of the subject.

In still even a further embodiment, there is provided a method for treating or preventing an autoimmune disease in a patient comprising administering to said patient a therapeutically effective amount of a vaccine comprising an expression construct encoding a tumor associated HLA-A2-restricted peptide, wherein the HLA-A2-restricted peptide is a proteinase-3 peptide, a neutrophil elastase peptide, a cyclin E1 peptide, cyclin D, or a cyclin E2 peptide. The expression construct may be a non-viral expression construct or a viral expression construct.

In yet another embodiment, there is provided a method for preventing an autoimmune disease in a patient comprising (a) identifying a patient at risk of developing an autoimmune disease, and (b) administering to said patient a therapeutically effective amount of a vaccine comprising a HLA-A2-restricted peptide, wherein the HLA-A2-restricted peptide is a proteinase-3 peptide, a neutrophil elastase peptide, a myeloperoxidase peptide, a cyclin E1 peptide, cyclin D, or a cyclin E2 peptide.

In still yet a further embodiment, there is provided a method of generating HLA-restricted peptide specific cytotoxic T-lymphocytes (CTLs) comprising (a) obtaining a target peptide pulsed onto antigen-presenting cells to expand the CTL ex vivo; (b) concentrating low affinity HLA-restricted peptide specific CTLs; and (c) expanding the concentrated HLA-restricted peptide specific CTLs. The target peptide may be provided in a limited amount such as at least 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 15, 20, 25, 30, 35, 40, 45, 50 60, 70, 80, 90, 100, 100, 110, 120, 130, 140, 150,160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, or more μM.

The method may further comprise purifying the concentrated HLA-restricted peptide specific CTLs. Step (b) may occur prior to, after, or both before and after purifying the concentrated HLA-restricted peptide-specific CTLs. Purifying may comprise high-speed flow cytometry. The target peptide may depend upon the donor and the peptide epitope. Concentrating low affinity HLA-restricted peptide specific CTLs comprises using microbeads and/or high speed sorting. The method may further comprise concentrating high affinity HLA-restricted peptide specific CTLs, and may even further comprise expanding the concentrated high affinity HLA-restricted peptide specific CTLs. The method may also further comprising adoptive T cell immunotherapy, and may also further comprise expansion of adoptively transferred peptide-specific T lymphocytes, and may also further comprise vaccinating the recipient with the specific peptide combined with an adjuvant after the transfer of the peptide-specific T lymphocytes.

In still a further embodiment, there is provided a method of generating HLA-restricted peptide specific regulatory T-cells comprising (a) obtaining a target peptide pulsed onto antigen-presenting cells to expand the T-cells ex vivo; (b) concentrating HLA-restricted peptide specific regulatory T-cells; and (c) expanding the concentrated HLA-restricted peptide specific regulatory T-cells.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Peptide-specific cytotoxicity of CTL against peptide-loaded T2 cells. Effector cells were plated with target cells in a 4-hr cytotoxicity assay at E:T ratios from 50:1 to 6:1. CTL raised against T2 cells pulsed with PR-1 or PR-2 were tested for specific lysis against the same respective target cell/target peptide combination. Six replicate wells were used for each dilution of effector cells. Data were pooled from 3 separate experiments using 3 separate CTL lines and displayed as mean specific lysis ± standard deviation.

FIGS. 2A-2B. PR1 specific CTL preferentially lyse fresh myeloid leukemia cells. CTL effector cells were plated with target cells in a 4-hr cytotoxicity assay at E:T ratios from 50:1 to 6:1. Six replicate wells were used for each dilution of effector cells. Data were pooled from 3 separate experiments using 3 separate CTL lines and displayed as mean specific lysis ± standard deviation. FIG. 2A—CTL specific for PR-1 demonstrating low specific lysis against K562 cells transfected with HLA-A2.1 (low proteinase 3 expression) and T2 cells loaded with PR-1 (positive controls). There was only background lysis against U 937 (low proteinase 3 expression, HLA-A2.1 negative) and T2 cells not loaded with peptide (negative controls). FIG. 2B—CTL specific for PR-1 demonstrate high specific lysis against marrow cells from patients with chronic myelogenous leukemia in chronic phase (CML-CP), accelerated phase (CML-AP), blast crisis (CML-BC), acute myelogenous leukemia (M4 subtype), and only background lysis toward marrow cells from a healthy marrow donor (D2) for patient P3.

FIG. 3. HMY-A2.1+ cells transfected with Pr3 gene express cytoplasmic protein. HMy2.CIR-A2 cells were transfected with the Pr3-containing vector pRTZ.2 and grown in culture in the presence of Zeocin. Cells were then grown in limiting dilution using increasingly higher concentrations of Zeocin. Two resulting cell lines, Clone 2.2 and Clone 1.4, and the non-transfected parent cell line HMy2.CIR-A2, were then analyzed for cytoplasmic Pr3 expression by flow cytometry using indirect staining with a FITC-labeled secondary antibody. Histograms of cell number versus the median channel of fluorescence (MCF) intensity of staining for cytoplasmic Pr3 are shown.

FIG. 4. PR1 specific CTL lyse Pr3-transfected HMY-A2.1+ cells. CTL effector cells were plated with target cells in a 4-hr cytotoxicity assay at E:T ratio of 25:1. Six replicate wells were used, and data is displayed as mean specific lysis ± standard deviation. CTL specific for PR-1 demonstrate 36% specific lysis of HMy2.CIR-A2 cells transfected with Pr3 (Clone 1.4), and 73% specific lysis of marrow cells from a patient with chronic myelogenous leukemia in blast crisis (CML-BC), (positive control). There was only background lysis of HL-60 cells (low proteinase 3 expression, HLA-A2.1 negative), non-Pr3 transfected HMy2.CIR-A2 cells, CAT transfected HMy2.CIR cells, and marrow cells from a normal donor (negative controls).

FIGS. 5A-5C. PR1-specific CTL can be identified by a PR1-tetramer. FIG. 5A—A day 17 CTL line elicited against PR1 was labeled for 30 min with anti-CD8-FITC, washed three times with PBS, and then labeled for 30 min with the PR1-tetramer-PE. Cells were then washed three more times with PBS and analyzed by flow cytometry. 2.4% of the CTL line was specific for PR1. FIG. 5B—An HLA-A2 tetramer specific for a new shock peptide stained 0.2% of the PR1-specific CTL. FIG. 5C—A day 17 CTL line elicited against influenza nucleoprotein was also labeled with anti-CD8-FITC and the PR1-tetramer-PE and is shown as a negative control.

FIG. 6. The PR-tetramer can be used to sort CTL with PR1-specificity. A day 32 CTL line elicited against PR1 was stained for 2 hr with anti-CD8-FITC, washed 3 times with PBS, then labeled for 2 hr on ice with the PR1-tetramer-PE conjugate. Cells were then labeled a third time with anti-CD4 and PI, washed 3 times with PBS, and sorted for dual-staining CD8+PR1-tetramer positive cells. FL1-H is CD8-FITC and FL2-H is PR1-tetramer-PE. The CTL line following sorting is shown.

FIG. 7. CTL sorted for PR1-specificity show higher specific lysis of leukemia targets than non-sorted bulk culture CTL, with less background lysis. Day 32 bulk culture PR1-pulsed CTL were compared to PR1-tetramer-sorted CTL as effector cells plated with marrow cells from an HLA-A2.1+ patient with CML in accelerated phase (CML BM) or marrow cells from an HLA identical normal donor (Normal BM). CTL were derived from an unrelated normal donor matched only for HLA-A2.

FIG. 8. SSCP analysis of the 5 PR3 exons. Shown here are the best results obtained for each exon (Ex 1 to 5) in 10 pairs of donor-recipient (D-R) pairs. Arrowheads indicated bands present in the recipient but not in the donor.

FIG. 9. Pr3 exon 3 containing polymorphism codes for peptides that bind to HLA-A2.1. Surface HLA-A2.1 expression after 18 hr of incubation of T2 cells with 100 microgram of either PR71, PR7V, or control influenza peptide. Isotype control is shown at far left.

FIG. 10. Tissue Expression of Proteinase 3.

FIGS. 11A-11D. HLA-A*0201 Expression on T2. Lower doses of PR1 peptide induce CTLs with higher intensity PR1/HLA-A2 tetramer staining that correlates with TCR avidity and inversely with effector function threshold. FIG. 11A—PBMCs collected from healthy HLA-A2.1⁺ donors were stimulated weekly with PR1 peptide-pulsed T2 cells at the peptide concentrations indicated above each FACS plot. After 4 weeks, resulting cultures were stained with CD8 (FITC) Ab and PR1/HLA-A2 tetramer, and the percentage of CD8⁺ cells that stain with tetramer are noted within each FACS plot. FIG. 11B—Surface HLA-A2 expression on T2 cells increases linearly with increasing concentration of PR1 peptide from 2 μM and 200 μM. T2 cells were incubated with PR1 peptide at the concentrations shown and surface HLA-A2 expression was measured by flow cytometry. FIG. 11C—CTLs elicited with PR1 at 0.2 μM (open circles) or 20 μM (filled squares) PR1 were incubated for 4 hr at 37° C. with PR1 peptide-pulsed T2 cells at the indicated peptide concentrations at an effector/target (E/T) ratio of 10:1 (adjusted based on the number of tetramer-positive CTLs), and percentage of specific lysis was determined. FIG. 11D—Tetramer decay (t_{1/2}) was determined to be 58 min and 19 min by plotting normalized antigen-specific fluorescence at the indicated time points for 28-day-old PR1/HLA-A2 tetramer-stained CTLs elicited with 0.2 μM (open circles) or 20 μM PR1 (filled squares), respectively. Dissociation kinetics of PR1/HLA-A2 tetramer staining were determined at 4° C. in the presence of saturating concentrations of BB7.2 Ab to prevent rebinding of tetramer and in the presence of PI (1 μg/ml) to eliminate dead cells from the FACS gate.

FIGS. 12A-12B. Spectratyppe from high and low avidity PR1-specific CTL cultures. Vβ2 and Vβ11 at day 0 and day 26 are shown. FIG. 12A—0.2 μM PR1. FIG. 12B—20 μPR1.

FIGS. 13A-13B. High-avidity PR1-specific CTLs cause more specific lysis of CML BM cells than low-avidity PR1-specific CTLs. After 4 weeks in culture, PR1-stimulated CTLs were coincubated in a 4-hr microcytotoxicity assay with bone marrow cells, and specific lysis was determined. Six replicate wells were used for each dilution of effector cells. Specific lysis is plotted versus E/T ratio, and effector number was normalized for the number of PR1/HLA-A2 tetramer-staining cells in the bulk culture. FIG. 13A—High-avidity PR1-specific CTLs from a healthy donor showed greater specific lysis of CML target cells than low-avidity PR1-specific CTLs. FIG. 13B—PR1-specific CTL line from a CML patient 3 months after IFN treatment preferentially lyse autologous BM target cells taken at time of diagnosis over healthy HLA-A2⁺ BM cells from a third party, and the amount of CML target cell lysis is similar to that produced by healthy donor-derived low-avidity PR1-specific CTLs.

FIGS. 14A-14C. Only low-avidity PR1-specific CTLs are elicited from peripheral blood of CML patients. PBMCs from three different HLA-A2⁺ CML patients were stimulated weekly with PR1-pulsed T2 cells with PR1 ranging from 0.002 μM to 200 μM. After 4 weeks, resultant cultures were stained with CD8⁺ Ab and PR1/HLA-A2 tetramer and analyzed by FACS. The percentage of CD8⁺ cells that stain with relevant tetramer is indicated within each FACS plot. FIG. 14A—Cultures elicited with 0.2 μM, 0.02 μM, and 0.002 μM PR1 resulted in CTLs with lower-intensity tetramer staining than CTLs from healthy donors elicited with similar doses of PR1. FIG. 14B—PBMCs from an untreated chronic phase CML patient (CML no. 4) were studied weekly prior to restimulation with PR1-pulsed T2 cells with PR1/HLA-A2 tetramer. Only PR1-specific CTLs with low-intensity tetramer staining emerge over the 4 weeks, and no relatively high tetramer intensity CTLs are present. FIG. 14C—PBMCs from CML no. 2 stimulated weekly with 0.2 μM pp65 peptide elicited CTLs with high-intensity pp65/HLA-A2 tetramer staining after 4 weeks in culture.

FIGS. 15A-15C. High-avidity PR1-specific CTLs are identified in the peripheral blood of IFN sensitive CML patients (FIG. 15A) in cytogenetic remission, but not in (FIG. 15B) IFN-resistant or in (FIG. 15C) untreated newly diagnosed CML patients. PBMCs were stained with CD8, dump (CD14+CD19), and PR1-HLA-A2 tetramer. Patients 5-8 were treated for a minimum of 9 months with IFN. The percentage of Ph⁺ chromosomes in a simultaneous BM specimen is indicated above each FACS plot, and the percentage of CD8⁺ cells with high-avidity PR1-specific CTLs is indicated within each FACS plot.

FIGS. 16A-16B. High-avidity PR1 -specific CTLs undergo apoptosis 18 hr after stimulation with high-concentration PR1 peptide. PBMCs from a healthy donor 28 days after weekly restimulation with either 0.2 μM or 20 μM PR1-pulsed T2 cells established relatively high- and low-avidity PR1-CTL, respectively (far left panels). The resulting PR1-CTLs were washed and combined in a 1:1 ratio, based on the number of tetramer-positive cells, with T2 cells pulsed with either 0.2 μM or 20 μM PR1 peptide. After 16 to 18 hr, cells were stained with Annexin V Ab, and live cells were analyzed based on PI staining. The percentage of CD8⁺ cells that are tetramer-positive is shown in the far left panels, and the percentage of tetramer-positive cells that stain with annexin V are shown in the remaining panels. FIG. 16A—Annexin V expression increased on high-avidity PR1-CTLs exposed to high-concentration (20 μM) PR1, but not after exposure to low (0.2 μM) concentration PR1. Annexin V upregulation was blocked by pretreating peptide-pulsed T2 cells with anti-HLA-A2 (BB7.2) prior to coculture with PR1-CTL. FIG. 16B—Annexin V was not upregulated 18 hr after coculture of low-avidity PR1-CTLs with either low-concentration (0.2 μM) or high concentration (20 μM) PR1 peptide.

FIGS. 17A-17D. High-avidity PR1-CTLs undergo apoptosis 18 hr after coincubation with HLA-A2⁺ CML cells that overexpress proteinase 3. High- and low-avidity PR1-CTLs were combined in a 1:1 ratio, based upon the number of tetramer-positive cells, with CML BM cells from untreated HLA-A2⁺ and HLA-A2⁻ patients. Annexin V staining was measured on live cells, based on PI staining, 18 hr after coincubation. The percentage of CD8⁺ cells that are tetramer-positive is shown in the left panels, and the percentage of tetramer-positive cells that stain with annexin V are shown in the remaining panels. FIG. 17A—Annexin V was upregulated in the high-avidity PR1-CTLs after coincubation with HLA-A2⁺ cells, but not after coincubation with HLA-A2-cells. Remaining low-avidity PR1-CTLs in the culture did not upregulate annexin V. FIG. 17B—In contrast, low-avidity PR1-CTLs did not upregulate annexin V after coincubation with either HLA-A2⁺ or HLA-A2⁻ CML BM cells. FIG. 17C—Overall MHC-I expression and proteinase 3 expression was similar in both CML BM target cells, as measured by surface staining with pan-HLA-A,B,C Ab. FIG. 17D—Proteinase 3 expression was 2.8- and 3.3-fold higher in the HLA-A2⁺ and the HLA-A2⁻ patient BM, respectively, compared with healthy donor BM cells.

FIG. 18. High avidity PR1-CTL in IFN-sensitive CML patients off therapy.

FIG. 19. PR1-CTL phenotype in CML patients in CCR off interferon: High avidity PR1-CTL have an effector memory phenotype.

FIG. 20. PR1 vaccine elicits PR1-CTL immunity at injection site.

FIG. 21. PR1 vaccine induces immune responses and clinical responses.

FIG. 22. PR1 vaccine induces PR1-CTL immunity and molecular remission in UPN4 with AML. The fraction of functional PR1-CTL vs pp65-CTL is similar in CMV⁺ patients with an immune response to the PR1 vaccine.

FIG. 23. PR1 vaccine elicits functional PR1-CTL immunity that persiste beyond 6 months. UPN9: PBMC analyzed six months after 3^{rd} injection of PR1 (1.0 mg) for tetramer⁺ and CD69/IFN⁺ PR1-CTL.

FIGS. 24A-24B. In UPN4 patient with AML t(15;17) long term molecular remission after vaccine-induced expansion of PR1-CTL was test (FIG. 24A) and vaccine-induced PR1-CTL was shown to preferentially kill leukemia cells (FIG. 24B).

FIG. 25. PR1 vaccine induces molecular remission in UPN15 patient with inv(16) AML.

FIG. 26. Clinical response correlates with immune response and higher T Cell Receptor avidity of PR1-CTL. The data shows 5 clinical responses in 8 patients with immune response versus 0 clinical responses in 7 patients without immune response. p=0.02.

FIGS. 27A-27D. High avidity PR1-CTL are preferentially lost after vaccination and remaining low avidity PR1-CTL specifically kill autologous leukemia cells in UPN6 patient. FIG. 27A—Using sample from patient 6 (UPN 6) percent antigens specific CTL was assessed. FIG. 27B—Pre and Post-vaccine. FIG. 27C—PR1/A2 Tetramer-Sorted CTL Are Peptide Specific. FIG. 27D—Tetramer-Sorted PR1-CTL Preferentially Kill CML.

FIG. 28. High affinity PR1-CTL are absent in UPN 6 after vaccination. UPN6 (CML) and UPN4 (AML) patient samples are compared 30 days post-PR1 at 200 μM and 0.2 μM. High PR1-CTL avidity was found to be expanded in UPN4 but not UPN61 patient.

FIGS. 29A-29B. PR1-CTL immunity at day 60 correlates with remission at 1 year after NST for AML.

FIG. 30. Efficiency of various APC populations to elicit CTL. Cell count comparison of CTL cultures elicited with PR1-pulsed T2 DC/IGM or DC/4GM antigen presenting cells.

FIGS. 31A-31B. Cytotoxicity comparison of PR1-CTL elicited with various APCs. Percent specific lysis for T2 alone versus T2+PR1 in DC/IGM (FIG. 31A) or DC/4GM (FIG. 31B) antigen presenting cells is shown.

FIG. 32. Comparison of DC growth conditions on CTL stimulation. Cell proliferation of PR1-CTL lines stimulated with autologous DC/IGM under various culture conditions is shown.

FIGS. 33A-33C. IFN-Induced P3 expression correlates with PR1-CTL response and CCR to IFN.

FIG. 34. Five MPO-Deduced Peptides Bind to HLA-A2.1.

FIG. 35. MY2-Specific CTL Kill Leukemia Cells and Also Healthy Donor Marrow Cells.

FIG. 36. MY4-Sepcific CTL Preferentially Kill AML Bone Marrow Cells And Not Healthy Bone Marrow Cells.

FIG. 37. MY4-Specific CTL Lysis of MY4-Pulsed T2 Cells is HLA-A2-Restricted.

FIG. 38. MY4-CTL Inhibit Leukemia Progenitors.

FIG. 39. MPO Protein is Present in CD34⁺ Leukemia Cells, but not Normal CD34⁺ Cells.

FIG. 40. Tissue Expression of Myeloperoxidase.

FIG. 41. Multiple Peptide/HLA-A2-Tetramers Can Be Used To Simultaneously Stain Single Patient Samples.

FIG. 42. PR1-, MY2- & MY4-CTL Are Not Present In Healthy Donors Or NST Recipients With Lymphoid Malignancies.

FIG. 43. PR1-CTL & MY4-CTL, But No MY2-CTL, Are Detectable In Nonmyeloablative Stem Cell Transplant (NST) Recipients In Complete Remission.

FIG. 44. Percentage of PR1-TL, MY4-CTL & HA1-CTL All Correlate with Remission Status.

FIG. 45. PR1-CTL Kill Leukemia but not Normal Marrow.

FIG. 46. PR1-CTL Lysis of Targets is HLA-A2-Restricted.

### DESCRIPTION OF ILLUSTRATIVE EMBODIMENTS

### I. THE PRESENT INVENTION

The present invention serves to overcome the deficiencies in the art by providing HLA-restricted peptides, derived from myeloid self-proteins, that can be used to elicit peptide-reactive CTL that are useful in treating treating or preventing of autoimmune diseases/conditions, transplant rejection and vasculitis.

The findings of the present invention suggest that the PR1 peptide is an important tumor antigen for CTL immune responses against this form of leukemia, and provide the first direct evidence that an antigen-specific T cell response contributes to its control. PR1-specific CTL are found in the majority of patients that achieve remission with IFN-α2b therapy or with allogeneic BMT, therapies thought to potentially work through an immune mechanism. The inventors have shown that normal healthy donors have existent CTL immunity to PR1 and that CML patients who have a cytogenetic remission after treatment with interferon also have effective PR1-specific CTL immunity toward their leukemia cells, while patients without cytogenetic responses do not, thus establishing PR1 as the first leukemia-associated tumor antigen. The present invention also seeks to determine whether vaccination with PR1 peptide can enhance immunity toward leukemia, whether the responding T lymphocytes are memory or naive, and whether the PR1-specific CTL exhibit T cell receptors (TCRs) with higher or lower affinity for PR1/HLA-A2 compared to healthy individuals.

In other aspects of the present invention the in vivo relevance of MY2 and MY4 as leukemia antigens is examined since the peptides were identified using in vitro techniques. Pre-existing MY2 and MY4 reactivity by CTL in leukemia patients and their HLA-matched marrow donors will also be examined. The present invention will determine whether MY2- and MY4-specific CTL can be elicited in vitro from leukemia patients using the established methods for eliciting similar responses in normal donors. In the present invention, there is also provided a phase I clinical trial to adoptively transfer MY4-specific CTL to acute myeloid leukemia patients following NST as a way to enhance GVL while abrogating GVHD. Ways to enhance MY4 responses in vitro will also be assessed, and the remainder of the HLA-A2.1-restricted, HLA-A3-restricted, and HLA-B7-restricted peptides from MPO with predicted high MHC binding will be examined for their capacity to elicit myeloid leukemia-reactive CTL responses similar to MY4 and PR1.

### II. DEFINITIONS

The phrases “isolated” or “biologically pure” refer to material which is substantially or essentially free from components which normally accompany the material as it is found in its native state. Thus, isolated peptides in accordance with the invention preferably do not contain materials normally associated with the peptides in their in situ environment.

“Major histocompatibility complex” or “MHC” is a cluster of genes that plays a role in control of the cellular interactions responsible for physiologic immune responses. In humans, the MHC complex is also known as the HLA complex. For a detailed description of the MHC and HLA complexes (see Paul, 1993).

“Human leukocyte antigen” or “HLA” is a human class I or class II major histocompatibility complex (MHC) protein (see, e.g., Stites, 1994).

An “HLA supertype or family”, as used herein, describes sets of HLA molecules grouped on the basis of shared peptide-binding specificities. HLA class I molecules that share somewhat similar binding affinity for peptides bearing certain amino acid motifs are grouped into HLA supertypes. The terms HLA superfamily, HLA supertype family, HLA family, and HLA xx-like supertype molecules (where xx denotes a particular HLA type), are synonyms.

The term “motif” refers to the pattern of residues in a peptide of defined length, usually a peptide of from about 8 to about 13 amino acids for a class I HLA motif and from about 6 to about 25 amino acids for a class II HLA motif, which is recognized by a particular HLA molecule. Peptide motifs are typically different for each protein encoded by each human HLA allele and differ in the pattern of the primary and secondary anchor residues.

A “supermotif” is a peptide binding specificity shared by HLA molecules encoded by two or more HLA alleles. Thus, a preferably is recognized with high or intermediate affinity (as defined herein) by two or more HLA antigens.

“Cross-reactive binding” indicates that a peptide is bound by more than one HLA molecule; a synonym is degenerate binding. A “protective immune response” refers to a CTL and/or an HTL response to an antigen derived from an infectious agent or a tumor antigen, which prevents or at least partially arrests disease symptoms or progression. The immune response may also include an antibody response which has been facilitated by the stimulation of helper T cells.

### III. HLA-RESTRICTED PEPTIDES

The present provides a vaccine comprising a tumor associated HLA restricted peptide. “Human leukocyte antigen” or “HLA” is a human class I or class II major histocompatibility complex (MHC) protein (see, e.g., Stites, 1994). An “HLA supertype or family”, as used herein, describes sets of HLA molecules grouped on the basis of shared peptide-binding specificities. HLA class I molecules that share somewhat similar binding affinity for peptides bearing certain amino acid motifs are grouped into HLA supertypes. The terms HLA superfamily, HLA supertype family, HLA family, and HLA xx-like supertype molecules (where xx denotes a particular HLA type), are synonyms. HLA-restricted molecules of the present invention may include HLA-A2, HLA-A3, HLA-A11, HLA-B7, HLA-B27, or HLA-B35; but are not limited to such.

HLA-restricted antigens/peptides include, but are not limited to: 707 alanine proline (707-AP); alpha (α)-fetoprotein (AFP); adenocarcinoma antigen recognized by T cells 4 (ART-4); B antigen (BAGE); β-catenin/m, β-catenin/mutated; breakpoint cluster region-Abelson (Bcr-abl); CTL-recognized antigen on melanoma (CAMEL); carcinoembryonic antigen peptide-1 (CAP-1); caspase-8 (CASP-8); cell-division-cycle 27 mutated (CDC27m); cycline-dependent kinase 4 mutated (CDK4/m); carcinoembryonic antigen (CEA); cancer/testis (antigen) (CT); cyclophilin B (Cyp-B); differentiation antigen melanoma (the epitopes of DAM-6 and DAM-10 are equivalent, but the gene sequences are different. DAM-6 is also called MAGE-B2 and DAM-10 is also called MAGE-B1) (DAM); elongation factor 2 mutated (ELF2M); Ets variant gene 6/acute myeloid leukemia 1 gene ETS (ETV6-AM1); glycoprotein 250 (G250); G antigen (GAGE); N-acetylglucosaminyltransferase V (GnT-V); glycoprotein 100 Kd (Gp100); helicose antigen (HA GE); human epidermal receptor-2/neurological (HER-2/neu); arginine (R) to isoleucine (I) exchange at residue 170 of the α-helix of the α2-domain in the HLA-A2 gene (HLA-A*020]-R]701); human papilloma virus E7 (HPV-E7); heat shock protein 70-2 mutated (HSP70-2M); human signet ring tumor-2 (HST-2); human telomerase reverse transcriptase (hTERT or hTRT); intestinal carboxyl esterase (iCE); name of the gene as it appears in databases (KIAA0205); L antigen (LAGE); low density lipid receptor/GDP-L-fucose (LDLR/FUI); β-D-galactosidase 2-α-L-fucosyltransferase; melanoma antigen (MAGE); melanoma antigen recognized by T cells-1/Melanoma antigen A (MART-1/Melan-A); melanocortin 1 receptor (MC1R); myosin mutated (Myosinim); mucin 1 (MUCG); melanoma (MUM-1, -2, -3); ubiquitous mutated 1, 2, 3; NA cDNA clone of patient M88 (NA88-A); New York—esophageous 1 (NY-ESO-1); protein 15 (P15); protein of 190 (p190 minor bcr-abl); KD bcr-abl; promyelocytic leukaemia/retinoic acid receptor α (Pml/RARα); preferentially expressed antigen of melanoma (PRAME); prostate-specific antigen (PSA); prostate-specific membrane antigen (PSM); renal antigen (RAGE); renal ubiquitous 1 or 2 (RU1 or RU2); sarcoma antigen (SAGE); squamous antigen rejecting tumor 1 or 3 (SART-1 or SART-3); translocation Ets-family leukemia/acute myeloid leukemia 1 (TEL/AML1); triosephosphate isomerase mutated (TPI/m); tyrosinase related protein 1, or gp75 (TRP-1); tyrosinase related protein 2 (TRP-2); TRP-2/intron 2 (TRP-2/INT2); Wilms' tumor gene (WT1) or any such HLA-restricted antigen or peptide known to one of ordinary in the art.

In particular embodiments, the present invention contemplates the use of HLA-restricted peptide for treating diseases or conditions including but not limited to, autoimmune disorders, transplant rejections, or vasculitis.

### IV. MYELOID-RESTRICTED ANTIGENS

To adapt what has been learned about immunity against melanoma antigens to the study of myeloid leukemia antigens, myeloid-restricted normal proteins that are highly expressed in the leukemia are studied. Myeloid leukemias express a number of differentiation antigens associated with granule formation. These antigens may include proteinease-3 (Pr3 or P3), neutrophil elastase, myeloperoxidase, cylcin E1, cyclin D, or a cyclin E2; but are not limited to such. Particular examples of myeloid-restricted peptides, such as Pr3 and myeloperoxidase, are provided herein.

P3 and two other azurophil granule proteins, neutrophil elastase and azurocidin, are coordinately regulated and the transcription factors PU.1 and C/EBPα, which are responsible for normal myeloid differentiation from stem cells to monocytes or granulocytes, are important in mediating their expression (Lewin et al., 2002). These transcription factors have been implicated in leukemogenesis (Behre et al., 1999), and P3 itself may also be important in maintaining a leukemia phenotype since P3 antisense oligonucleotides halt cell division and induce maturation of the HL-60 promyelocytic leukemia cell line (Bories et al., 1989). Critical to identifying T cell antigens in these proteins is the observation that P3 is the target of autoimmune attack in Wegener's granulomatosis (Franssen et al., 1994).

A. Proteinase3 Peptides and Vaccines

Pr3 is a 26 kDa neutral serine protease that is stored in primary azurophil granules and is maximally expressed at the promyelocyte stage of myeloid differentiation (Sturrock et al., 1992; Chen et al., 1994; Muller-Berat et al., 1994; Lewin et al., 2002; Behre et al., 1999). The human gene contains 5 exons, is localized on chromosome 19p and has been cloned (Sturrock et al., 1992). Pr3 is overexpressed in a variety of myeloid leukemia cells including 75% of CML patients, approximately 50% of acute myeloid leukemia patients, and approximately 30% of the cases of myelodysplastic syndrome patients (Dengler et al., 1995).

Pr3 also has several characteristics that make it an appealing target for vaccine and T cell directed therapy. It is overexpressed in human myeloid leukemia and is generally homogeneous throughout the leukemia. An immune response generated against the antigen could result in complete eradication of the leukemia. Since Pr3 may be important for maintenance of the leukemia phenotype, any selective pressure resulting in Pr3-loss mutants following immunotherapy may not result in “tumor escape.” As a protein that is targeted to the endoplasmic reticulum by means of a pre-propeptide leader sequence, any TAP-deficient tumor mutants might remain susceptible to an anti-leukemia immune response since the protein would still be available to the MHC class I antigen processing pathway. Additionally, the use of a synthetic peptide derived from Pr3 as the immunizing antigen in a leukemia vaccine offers practical advantages: relatively easy construction and production, chemical stability, and a lack of infectious or oncogenic potential.

It has been shown that a small peptide called PR1, a portion of the larger molecule of proteinase 3 (P3) found in myeloid leukemia cells, can be used to generate immune cells, particularly, cytotoxic T lymphocytes (CTL). PR1 is a 9 aa peptide comprising amino acid 169-177, that binds to HLA-A2.1, thereby eliciting CTL from an HLA-A2.1+ normal donor in vitro. These PR1-specific CTL show preferential cytotoxicity toward allogeneic HLA-A2.1+ myeloid leukemia cells over HLA-identical normal donor marrow (Molldrem et al., 1996). PR1-specific CTL also inhibit colony-forming unit granulocyte-macrophage (CFU-GM) from the marrow of CML patients, but not CFU-GM from normal HLA-matched donors (Molldrem et al., 1997). These CTL, generated from normal healthy donors, preferentially kill leukemia cells while leaving normal bone marrow cells unharmed. More recently, it was found that CML patients who enter remission after treatment with either BMT or interferon have highly increased numbers of very effective PR1-specific CTL that kill their leukemia cells. PR1 is therefore the first peptide antigen identified that can elicit specific CTL lysis of fresh human myeloid leukemia cells.

B. Myeloperoxidase (MPO) Peptides and Vaccines

The invention also provides peptides of myeloperoxidase (MPO), another myeloid-restricted protein, which is a heme protein synthesized during early myeloid differentiation that constitutes the major component of neutrophil azurophilic granules. Produced as a single chain precursor, myeloperoxidase is subsequently cleaved into a light and heavy chain. The mature myeloperoxidase is a tetramer composed of 2 light chains and 2 heavy chains (Franssen et al., 1996). This enzyme produces hypohalous acids central to the microbicidal activity of netrophils. Importantly, MPO (like Pr3) is overexpressed in a variety of myeloid leukemia cells including 75% of CML patients, approximately 50% of acute myeloid leukemia patients, and approximately 30% of the cases of myelodysplastic syndrome patients (Williams et al., 1994). While Pr3 is the target of autoimmune attack in Wegener's granulomatosis MPO is the target antigen in small vessel vasculitis (Franssen et al., 1996; Brouwer et al., 1994; Molldrem et al., 1996) respectively, with evidence for both T-cell and antibody immunity in patients with these diseases. Wegener's granulomatosis is associated with production of cytoplasmic antineutrophil cytoplasmic antibodies (cANCA) with specificity for Pr3 (Molldrem et al., 1997), while microscopic polyangiitis and Churg-Strauss syndrome are associated with perinuclear ANCA (PANCA) with specificity for MPO (Molldrem et al., 1999; Savage et al., 1999). T-cells taken from such leukemia patients proliferate in response to crude extracts from neutrophil granules and to the purified proteins (Brouwer et al., 1994; Yee et al., 1999). These findings (summarized in Table 1) indicate that T cell responses against these proteins might be relatively easy to elicit.

TABLE 1
T cell Responses Myeloperoxidase (MPO)

Granule protein of 80 kD, gene on chromosome 17q
Target antigen of the pANCA antibody in small vessel
Vasculitis, Churg-Strauss syndrome, and crescentic Glomerulonephritis
Expressed in very early myeloid progenitors
Most abundant protein in myeloid cells
Aberrantly expressed in CD34+ myeloid leukemia cells (MDS, AML &
CML)

### V. HLA-RESTRICTED PEPTIDES AND VACCINES

In certain embodiments, the present invention concerns HLA-restricted peptide or antigen compositions comprising at least one HLA-restricted peptide, such as proteinase3 (P3 or Pr3) or myeloperoxidase (MYO) for use as a vaccine in treating autoimmune diseases or conditions, transplant rejection, or vasuclitis.

As used herein, an “antigenic composition” may comprise an antigen (e.g., a peptide or polypepide), a nucleic acid encoding an antigen (e.g., an antigen expression vector), or a cell expressing or presenting an antigen. For an antigenic composition, such as a tumor-associated HLA-restricted peptide or antigen of the present invention, to be useful as a vaccine, the antigenic composition must induce an immune response to the antigen in a cell, tissue or animal (e.g., a human). In particular embodiments, the antigenic composition comprises or encodes all or part of the sequences shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63 or an immunologically functional equivalent thereof.

As used herein, an “amino acid molecule” or “amino acid residue” refers to any naturally occurring amino acid, any amino acid derivative or any amino acid mimic known in the art, including modified or unusual amino acids. In certain embodiments, the residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties. In specific aspects, the composition of the present invention employs a peptide of from about 5 to about 100 amino acids or greater in length.

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases located at the National Institutes of Health website. The coding regions for known genes may be amplified and/or expressed using the techniques disclosed herein or as would be know to those of ordinary skill in the art. Alternatively, various commercial preparations of proteins, polypeptides and peptides are known to those of skill in the art.

The term “peptide” is used interchangeably with “oligopeptide” in the present specification to designate a series of residues, typically L-amino acids, connected one to the other, typically by peptide bonds between the a-amino and carboxyl groups of adjacent amino acids. The preferred CTL-inducing oligopeptides of the invention are 13 residues or less in length and usually consist of between about 8 and about 11 residues, preferably 9 or 10 residues. The preferred HTL-inducing oligopeptides are less than about 50 residues in length and usually consist of between about 6 and about 30 residues, more usually between about 12 and 25, and often between about 15 and 20 residues.

In certain embodiments the size of the at least one peptide molecule may comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, or greater amino molecule residues, and any range derivable therein.

An “immunogenic peptide” or “peptide epitope” is a peptide which comprises an allele-specific motif or supermotif such that the peptide will bind an HLA molecule and induce a CTL and/or HTL response. Thus, immunogenic peptides of the invention are capable of binding to an appropriate HLA molecule and thereafter inducing a cytotoxic T cell response, or a helper T cell response, to the antigen from which the immunogenic peptide is derived.

Accordingly, the term “proteinaceous composition” encompasses amino molecule sequences comprising at least one of the 20 common amino acids in naturally synthesized proteins, or at least one modified or unusual amino acid, including but not limited to those shown on Table 2 below.

TABLE 2
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | 2-alanine, -Amino-propionic acid | Ahyl | Allo-Hydroxylysine |
| Abu | 2-Aminobutyric acid | 3Hyp | 3-Hydroxyproline |
| 4Abu | 4-Aminobutyric acid, piperidinic | 4Hyp | 4-Hydroxyproline |
| | acid | | |
| Acp | 6-Aminocaproic acid | Ide | Isodesmosine |
| Ahe | 2-Aminoheptanoic acid | Aile | Allo-Isoleucine |
| Aib | 2-Aminoisobutyric acid | MeGly | N-Methylglycine, |
| | | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

In certain embodiments, the proteinaceous composition may comprise at least one antibody, for example, an antibody against PR1 or myeloperoxidase. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Harlow et al., 1988; incorporated herein by reference).

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

A. Fusion Proteins of HLA-Restiricted Peptides

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions.

B. Variants of HLA-Restiricted Peptides

It is contemplated that peptides of the present invention may further employ amino acid sequence variants such as substitutional, insertional or deletion variants. Deletion variants lack one or more residues of the native protein. Insertional mutants typically involve the addition of material at a non-terminal point in the polypeptide. -Substitutions are changes to an existing amino acid. These sequence variants may generate truncations, point mutations, and frameshift mutations. As is known to one skilled in the art, synthetic peptides can be generated by these mutations.

It also will be understood that amino acids sequence variants may include additional residues, such as additional N- or C-terminal amino acids, and yet still be essentially as set forth in one of the sequences disclosed herein, so long as the sequence meets the criteria set forth above, including the maintenance of biological activity.

The following is a discussion based upon changing the amino acids of a protein, such as a HLA-restricted peptide or protein of the invention, to create a mutated, truncated, or modified protein. For example, certain amino acids may be substituted for other amino acids in the tumor-associated HLA-restricted peptide or protein such as a Pr3 or MYO protein, resulting in a greater CTL immune response in cells such as a myeloid cell. Since it is the interactive capacity and nature of a protein that defines that protein's biological functional activity, certain amino acid substitutions can be made in a protein sequence, and in its underlying nucleic acid coding sequence, thereby producing a mutated, truncated or modified protein.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Patent 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present invention may also employ the use of peptide mimetics for the preparation of polypeptides (see e.g., Johnson, 1993) having many of the natural properties of a tumor-associated HLA-restricted peptide such as Pr3 or MYO protein, but with altered and/or improved characteristics. The underlying rationale behind the use of peptide mimetics is that the peptide backbone of proteins exists chiefly to orient amino acid side chains in such a way as to facilitate molecular interactions, such as those of antibody and antigen. These principles may be used, in conjunction with the principles outline above, to engineer second generation molecules having many of the natural properties of a tumor-associated HLA-restricted peptide but with altered and even improved characteristics.

C. HLA-Restricted Peptide Purification

In certain embodiments the protein(s) of the present invention may be purified. It may be desirable to purify the HLA-restricted peptides, polypeptides or proteins or variants thereof. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an HLA-restricted peptide of the present invention, it may be desirable to express the polypeptide in a prokaryotic or eukaryotic expression system and extract the protein using denaturing conditions. The polypeptide may be purified from other cellular components using an affinity column, which binds to a tagged portion of the polypeptide. Although this preparation will be purified in an inactive form, the denatured material will still be capable of transducing cells. Once inside of the target cell or tissue, it is generally accepted that the polypeptide will regain full biological activity.

As is generally known in the art, it is believed that the order of conducting the various purification steps may be changed, or that certain steps may be omitted, and still result in a suitable method for the preparation of a substantially purified protein or peptide.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity, herein assessed by a “—fold purification number.” The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### VI. HLA-RESTRICTED ANTIGENIC SEQUENCES

It is also contemplated in the present invention that peptides corresponding to one or more antigenic determinants of the HLA-restricted peptides or polypeptides may be prepared so that an immune response against the HLA-restricted peptides, polypeptides or proteins, such as Pr3 or MYO is raised. Thus, it is contemplated that vaccination with an HLA-restricted peptide, or polypeptide, may generate an autoimmune response in an immunized animal such that autoantibodies that specifically recognize the animal's endogenous HLA-restricted protein. This vaccination technology is shown in U.S. Pat. Nos. 6,027,727; 5,785,970, and 5,609,870, which are hereby incorporated by reference.

Such peptides should generally be at least five or six amino acid residues in length and will preferably be about 10, 11, 12, 13, 14, 15, 16, 17, 18, 19, 20, 25 or about 30 amino acid residues in length, and may contain up to about 35-50 residues. For example, these peptides may comprise a amino acid sequence, such as 5, 6, 7, 8, 9, 10, 11, 12, 13, 14, 15, 16, 17, 18, 19, 20, 21, 22, 23, 24, 25, 30, 35, 40, 45, and 50 or more contiguous amino acids from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:42, SEQ ID NO:43, SEQ ID NO:44, SEQ ID NO:45, SEQ ID NO:46, SEQ ID NO:47, SEQ ID NO:48, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63. Synthetic peptides will generally be about 35 residues long, which is the approximate upper length limit of automated peptide synthesis machines, such as those available from Applied Biosystems (Foster City, Calif.). Longer peptides also may be prepared, e.g., by recombinant means.

U.S. Pat. No. 4,554,101, incorporated herein by reference, teaches the identification and preparation of epitopes from primary amino acid sequences on the basis of hydrophilicity. Through the methods disclosed in Hopp, one of skill in the art would be able to identify epitopes from within an amino acid sequence such as the tumor-associated HLA-restricted peptides sequences disclosed herein in SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:42, SEQ ID NO:43, SEQ ID NO:44, SEQ ID NO:45, SEQ ID NO:46, SEQ ID NO:47, SEQ ID NO:48, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63 or fragments thereof.

Numerous scientific publications have also been devoted to the prediction of secondary structure, and to the identification of epitopes, from analyses of amino acid sequences (Chou and Fasman, 1974a,b; 1978a,b; 1979). Any of these may be used, if desired, to supplement the teachings of Hopp in U.S. Pat. No. 4,554,101.

Moreover, computer programs are currently available to assist with predicting antigenic portions and epitopic core regions of proteins. Examples include those programs based upon the Jameson-Wolf analysis (Jameson and Wolf, 1988; Wolf et al., 1988), the program PepPlotg (Brutlag et al., 1990; Weinberger et al., 1985), and other new programs for protein tertiary structure prediction (Fetrow and Bryant, 1993). Another commercially available software program capable of carrying out such analyses is MacVector (IBI, New Haven, Conn.).

In further embodiments, major antigenic determinants of a tumor-associated HLA-restricted peptide may be identified by an empirical approach in which portions of the gene encoding the tumor-associated HLA-restricted peptides are expressed in a recombinant host, and the resulting proteins tested for their ability to elicit an immune response. For example, PCR™ can be used to prepare a range of peptides lacking successively longer fragments of the C-terminus of the protein. The immunoactivity of each of these peptides is determined to identify those fragments or domains of the polypeptide that are immunodominant. Further studies in which only a small number of amino acids are removed at each iteration then allows the location of the antigenic determinants of the polypeptide to be more precisely determined.

Another method for determining the major antigenic determinants of a polypeptide is the SPOTs system (Genosys Biotechnologies, Inc., The Woodlands, Tex.). In this method, overlapping peptides are synthesized on a cellulose membrane, which following synthesis and deprotection, is screened using a polyclonal or monoclonal antibody. The antigenic determinants of the peptides which are initially identified can be further localized by performing subsequent syntheses of smaller peptides with larger overlaps, and by eventually replacing individual amino acids at each position along the immunoreactive peptide.

Once one or more such analyses are completed, polypeptides are prepared that contain at least the essential features of one or more antigenic determinants. The peptides are then employed in the generation of antisera against the polypeptide. Minigenes or gene fusions encoding these determinants also can be constructed and inserted into expression vectors by standard methods, for example, using PCR™ cloning methodology.

The use of such small peptides for antibody generation or vaccination typically requires conjugation of the peptide to an immunogenic carrier protein, such as hepatitis B surface antigen, keyhole limpet hemocyanin or bovine serum albumin, or other adjuvants discussed above (adjuvenated peptide). Alum is an adjuvant that has proven sufficiently non-toxic for use in humans. Methods for performing this conjugation are well known in the art. Other immunopotentiating compounds are also contemplated for use with the compositions of the invention such as polysaccharides, including chitosan, which is described in U.S. Pat. No. 5,980,912, hereby incorporated by reference. Multiple (more than one) tumor-associated HLA-restricted epitopes may be crosslinked to one another (e.g., polymerized). Alternatively, a nucleic acid sequence encoding a tumor-associated HLA-restricted peptides, or polypeptides may be combined with a nucleic acid sequence that heightens the immune response. Such fusion proteins may comprise part or all of a foreign (non-self) protein such as bacterial sequences, for example.

One of ordinary skill would know various assays to determine whether an immune response against an HLA-restricted peptide was generated. The phrase “immune response” includes both cellular and humoral immune responses. Various B lymphocyte and T lymphocyte assays are well known, such as ELISAs, cytotoxic T lymphocyte (CTL) assays, such as chromium release assays, proliferation assays using peripheral blood lymphocytes (PBL), tetramer assays, and cytokine production assays. See Benjamini et al. (1991), hereby incorporated by reference.

### VII. VACCINE COMPONENTS

In other embodiments of the invention, the antigenic composition, such an HLA-restricted peptide or antigen, may comprises an additional immunostimulatory agent or nucleic acids encoding such an agent. Immunostimulatory agents include but are not limited to an additional antigen, an immunomodulator, an antigen presenting cell or an adjuvant. In other embodiments, one or more of the additional agent(s) is covalently bonded to the antigen or an immunostimulatory agent, in any combination. In certain embodiments, the antigenic composition is conjugated to or comprises an HLA anchor motif amino acids.

A. Adjuvants

As also well known in the art, the immunogenicity of a particular immunogen composition can be enhanced by the use of non-specific stimulators of the immune response, known as adjuvants. Adjuvants have been used experimentally to promote a generalized increase in immunity against unknown antigens (e.g., U.S. Pat. No. 4,877,611). Immunization protocols have used adjuvants to stimulate responses for many years, and as such adjuvants are well known to one of ordinary skill in the art. Some adjuvants affect the way in which antigens are presented. For example, the immune response is increased when protein antigens are precipitated by alum. Emulsification of antigens also prolongs the duration of antigen presentation. The use of the HLA-restricted antigens of the present invention with the inclusion of a suitable adjuvant will likely increase the anti-tumor response of the antigens. Suitable molecule adjuvants include all acceptable immunostimulatory compounds, such as cytokines, toxins or synthetic compositions.

Exemplary, often preferred adjuvants include complete Freund's adjuvant (a non-specific stimulator of the immune response containing killed Mycobacterium tuberculosis), incomplete Freund's adjuvants and aluminum hydroxide adjuvant. Other adjuvants that may also be used include IL-1, IL-2, IL-4, IL-7, IL-12, -interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MHC antigens may even be used.

In one aspect, an adjuvant effect is achieved by use of an agent, such as alum, used in about 0.05 to about 0.1% solution in phosphate buffered saline. Alternatively, the antigen is made as an admixture with synthetic polymers of sugars (Carbopolo) used as an about 0.25% solution. Adjuvant effect may also be made my aggregation of the antigen in the vaccine by heat treatment with temperatures ranging between about 70° to about 101° C. for a 30 second to 2-minute period, respectively. Aggregation by reactivating with pepsin treated (Fab) antibodies to albumin, mixture with bacterial cell(s) such as C. parvum, an endotoxin or a lipopolysaccharide component of Gram-negative bacteria, emulsion in physiologically acceptable oil vehicles, such as mannide mono-oleate (Aracel A), or emulsion with a 20% solution of a perfluorocarbon (Fluosol-DA®) used as a block substitute, also may be employed.

Some adjuvants, for example, certain organic molecules obtained from bacteria, act on the host rather than on the antigen. An example is muramyl dipeptide (N-acetylmuramyl-L-alanyl-D-isoglutamine [MDP]), a bacterial peptidoglycan. The effects of MDP, as with most adjuvants, are not fully understood. MDP stimulates macrophages but also appears to stimulate B cells directly. The effects of adjuvants, therefore, are not antigen-specific. If they are administered together with a purified antigen, however, they can be used to selectively promote the response to the antigen.

In certain embodiments, hemocyanins and hemoerythrins may also be used in the invention. The use of hemocyanin from keyhole limpet (KLH) is preferred in certain embodiments, although other molluscan and arthropod hemocyanins and hemoerythrins may be employed.

Various polysaccharide adjuvants may also be used. For example, the use of various pneumococcal polysaccharide adjuvants on the antibody responses of mice has been described (Yin et al., 1989). The doses that produce optimal responses, or that otherwise do not produce suppression, should be employed as indicated (Yin et al., 1989). Polyamine varieties of polysaccharides are particularly preferred, such as chitin and chitosan, including deacetylated chitin.

Another group of adjuvants are the muramyl dipeptide (MDP, N-acetylmuramyl-L-alanyl-D-isoglutamine) group of bacterial peptidoglycans. Derivatives of murarnyl dipeptide, such as the amino acid derivative threonyl-MDP, and the fatty acid derivative MTPPE, are also contemplated.

U.S. Pat. No. 4,950,645 describes a lipophilic disaccharide-tripeptide derivative of muramyl dipeptide which is described for use in artificial liposomes formed from phosphatidyl choline and phosphatidyl glycerol. It is the to be effective in activating human monocytes and destroying tumor cells, but is non-toxic in generally high doses. The compounds of U.S. Pat. No. 4,950,645 and PCT Patent Application WO 91/16347, are contemplated for use with cellular carriers and other embodiments of the present invention.

BCG (bacillus Calmette-Guerin, an attenuated strain of Mycobacterium) and BCG-cell wall skeleton (CWS) may also be used as adjuvants, with or without trehalose dimycolate. Trehalose dimycolate may be used itself. Trehalose dimycolate administration has been shown to correlate with augmented resistance to influenza virus infection in mice (Azuma et al., 1988). Trehalose dimycolate may be prepared as described in U.S. Pat. No. 4,579,945. BCG is an important clinical tool because of its immunostimulatory properties. BCG acts to stimulate the reticulo-endothelial system, activates natural killer cells and increases proliferation of hematopoietic stem cells. Cell wall extracts of BCG have proven to have excellent immune adjuvant activity. Molecular genetic tools and methods for mycobacteria have provided the means to introduce foreign genes into BCG (Jacobs et al., 1987; Snapper et al., 1988; Husson et al., 1990; Martinet al., 1990). Live BCG is an effective and safe vaccine used worldwide to prevent tuberculosis. BCG and other mycobacteria are highly effective adjuvants, and the immune response to mycobacteria has been studied extensively. With nearly 2 billion immunizations, BCG has a long record of safe use in man (Luelmo, 1982; Lotte et al., 1984). It is one of the few vaccines that can be given at birth, it engenders long-lived immune responses with only a single dose, and there is a worldwide distribution network with experience in BCG vaccination. An exemplary BCG vaccine is sold as TICE BCG (Organon Inc., West Orange, N.J.).

Amphipathic and surface active agents, e.g., saponin and derivatives such as QS21 (Cambridge Biotech), form yet another group of adjuvants for use with the immunogens of the present invention. Nonionic block copolymer surfactants (Rabinovich et al., 1994) may also be employed. Oligonucleotides are another useful group of adjuvants (Yamamoto et al., 1988). Quil A and lentinen are other adjuvants that may be used in certain embodiments of the present invention.

Another group of adjuvants are the detoxified endotoxins, such as the refined detoxified endotoxin of U.S. Pat. No. 4,866,034. These refined detoxified endotoxins are effective in producing adjuvant responses in mammals. Of course, the detoxified endotoxins may be combined with other adjuvants to prepare multi-adjuvant-incorporated cells. For example, combination of detoxified endotoxins with trehalose dimycolate is particularly contemplated, as described in U.S. Pat. No. 4,435,386. Combinations of detoxified endotoxins with trehalose dimycolate and endotoxic glycolipids is also contemplated (U.S. Pat. No. 4,505,899), as is combination of detoxified endotoxins with cell wall skeleton (CWS) or CWS and trehalose dimycolate, as described in U.S. Pat. Nos. 4,436,727, 4,436,728 and 4,505,900. Combinations of just CWS and trehalose dimycolate, without detoxified endotoxins, is also envisioned to be useful, as described in U.S. Pat. No. 4,520,019.

Those of skill in the art will know the different kinds of adjuvants that can be conjugated to cellular vaccines in accordance with this invention and these include alkyl lysophosphilipids (ALP); BCG; and biotin (including biotinylated derivatives) among others. Certain adjuvants particularly contemplated for use are the teichoic acids from Gram-cells. These include the lipoteichoic acids (LTA), ribitol teichoic acids (RTA) and glycerol teichoic acid (GTA). Active forms of their synthetic counterparts may also be employed in connection with the invention (Takada et al., 1995).

Various adjuvants, even those that are not commonly used in humans, may still be employed in animals, where, for example, one desires to raise antibodies or to subsequently obtain activated T cells. The toxicity or other adverse effects that may result from either the adjuvant or the cells, e.g., as may occur using non-irradiated tumor cells, is irrelevant in such circumstances.

Adjuvants may be encoded by a nucleic acid (e.g., DNA or RNA). It is contemplated that such adjuvants may be also be encoded in a nucleic acid (e.g., an expression vector) encoding the antigen, or in a separate vector or other construct. Nucleic acids encoding the adjuvants can be delivered directly, such as for example with lipids or liposomes.

B. Biological Response Modifiers

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/m2) (Johnson/Mead, N.J.), cytokines such as -interferon, IL-2, or IL-12 or genes encoding proteins involved in immune helper functions, such as B-7.

C. Chemokines

Chemokines, nucleic acids that encode for chemokines, and/or cells that express such also may be used as vaccine components. Chemokines generally act as chemoattractants to recruit immune effector cells to the site of chemokine expression. It may be advantageous to express a particular chemokine coding sequence in combination with, for example, a cytokine coding sequence, to enhance the recruitment of other immune system components to the site of treatment. Such chemokines include, for example, RANTES, MCAF, MIP1-alpha, MIP1-Beta, IP-10 and combinations thereof. The skilled artisan will recognize that certain cytokines are also known to have chemoattractant effects and could also be classified under the term chemokines.

D. Immunogenic Carrier Proteins

In certain embodiments, an antigenic composition may be chemically coupled to a carrier or recombinantly expressed with a immunogenic carrier peptide or polypetide (e.g., a antigen-carrier fusion peptide or polypeptide) to enhance an immune reaction. Exemplary and preferred immunogenic carrier amino acid sequences include hepatitis B surface antigen, keyhole limpet hemocyanin (KLH) and bovine serum albumin (BSA). Other albumins such as ovalbumin, mouse serum albumin or rabbit serum albumin also can be used as immunogenic carrier proteins. Means for conjugating a polypeptide or peptide to a immunogenic carrier protein are well known in the art and include, for example, glutaraldehyde, m-maleimidobenzoyl-N-hydroxysuccinimide ester, carbodiimide and bis-biazotized benzidine.

### VIII. ANTIBODIES AND ANTIBODY GENERATION

Another embodiment of the present invention are antibodies, in some cases, a human monoclonal antibody immunoreactive with the polypeptide sequence of an HLA-restricted peptide of the invention comprising SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:42, SEQ ID NO:43, SEQ ID NO:44, SEQ ID NO:45, SEQ ID NO:46, SEQ ID NO:47, SEQ ID NO:48, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63. It is also understood that this antibody is useful for screening samples from human patients for the purpose of detecting a particular HLA-restricted peptide present in the samples. The antibody also may be useful in the screening of expressed DNA segments or peptides and proteins for the discovery of related antigenic sequences. In addition, the antibody may be useful in passive immunotherapy for autoimmune diseases/conditions. All such uses of the antibodies and any antigens or epitopic sequences so discovered fall within the scope of the present invention.

Examples of other antibodies that may be employed in the present invention may include antibodies that react with T cells such as CD1, CD2, CD3, CD5, CD7 CD4, CD6, CD8 and CD27. Antibodies that react with myeloid cells may also be employed and include CD11b, CD11c, CD13, CD14, CD15, CD16, CD33, CD48, CD63, CD74, CD65, CD66, CD67 and CD68. Antibodies that react with undifferentiated cells may include HLA-DR, CD34 and CD38. It should be appreciated that multiple combinations of antibodies selected from the ones mentioned above are possible. Accordingly, it will be apparent to one skilled in the art that one can vary the antibody combinations In certain embodiments, the present invention involves antibodies. For example, all or part of a monoclonal, single chain, or humanized antibody may function as a vaccine for autoimmune disease/condition. Other aspects of the invention involve administering antibodies as a form of treatment or as a diagnostic to identify or quantify a particular polypeptide, such as tumor-associated HLA-restricted polypeptide, for example Pr3 or MYO polypeptide. As detailed above, in addition to antibodies generated against full length proteins, antibodies also may be generated in response to smaller constructs comprising epitopic core regions, including wild-type and mutant epitopes.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is may also be used to refer to any antibody-like molecule that has an antigen binding region, and includes antibody fragments such as Fab′, Fab, F(ab′)2, single domain antibodies (DABs), Fv, scFv (single chain Fv), and the like. The techniques for preparing and using various antibody-based constructs and fragments are well known in the art. Means for preparing and characterizing antibodies are also well known in the art (See, Harlow and Lane, 1988; incorporated herein by reference).

Monoclonal antibodies (mAbs) are recognized to have certain advantages, e.g., reproducibility and large-scale production, and their use is generally preferred. The invention thus provides monoclonal antibodies of the human, murine, monkey, rat, hamster, rabbit and even chicken origin.

The methods for generating monoclonal antibodies (mAbs) generally begin along the same lines as those for preparing polyclonal antibodies. Briefly, a polyclonal antibody may be prepared by immunizing an animal with an immunogenic polypeptide composition in accordance with the present invention and collecting antisera from that immunized animal. Alternatively, in some embodiments of the present invention, serum is collected from persons who may have been exposed to a particular antigen. Exposure to a particular antigen may occur a work environment, such that those persons have been occupationally exposed to a particular antigen and have developed polyclonal antibodies to a peptide, polypeptide, or protein. In some embodiments of the invention polyclonal serum from occupationally exposed persons is used to identify antigenic regions in the gelonin toxin through the use of immunodetection methods.

A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin also can be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection also may be given. The process of boosting and titering is repeated until a suitable titer is achieved. When a desired level of immunogenicity is obtained, the immunized animal can be bled and the serum isolated and stored, and/or the animal can be used to generate mAbs. mAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified polypeptide, peptide or domain, be it a wild-type or mutant composition. The immunizing composition is administered in a manner effective to stimulate antibody producing cells.

mAbs may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate mabs. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Humanized monoclonal antibodies are antibodies of animal origin that have been modified using genetic engineering techniques to replace constant region and/or variable region framework sequences with human sequences, while retaining the original antigen specificity. Such antibodies are commonly derived from rodent antibodies with specificity against human antigens. Such antibodies are generally useful for in vivo therapeutic applications. This strategy reduces the host response to the foreign antibody and allows selection of the human effector functions. “Humanized” antibodies are also contemplated, as are chimeric antibodies from mouse, rat, or other species, bearing human constant and/or variable region domains, bispecific antibodies, recombinant and engineered antibodies and fragments thereof. The techniques for producing humanized immunoglobulins are well known to those of skill in the art. For example U.S. Pat. No. 5,693,762 discloses methods for producing, and compositions of, humanized immunoglobulins having one or more complementarity determining regions (CDR's). When combined into an intact antibody, the humanized immunoglobulins are substantially non-immunogenic in humans and retain substantially the same affinity as the donor immunoglobulin to the antigen, such as a protein or other compound containing an epitope. Examples of other teachings in this area include U.S. Pat. Nos. 6,054,297; 5,861,155; and 6,020,192, all specifically incorporated by reference. Methods for the development of antibodies that are “custom-tailored” to the patient's disease are likewise known and such custom-tailored antibodies are also contemplated.

### IX. NUCLEIC ACIDS ENCODING HLA-RESTRICTED PROTEIN, PEPTIDES AND POLYPEPTIDES

It is contemplated in the present invention, that the HLA-restricted peptides, or polypeptides may be encoded by a nucleic acid sequence. A nucleic acid may be derived from genomic DNA, complementary DNA (cDNA) or synthetic DNA. Where incorporation into an expression vector is desired, the nucleic acid may also comprise a natural intron or an intron derived from another gene.

As used herein, the term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy. A tumor-associated HLA-restricted peptide or polypeptide cDNA, such as a Pr3 or MYO cDNA, for use in the present invention, may be derived from human cDNA but are not limited such.

As used herein, the term “nucleic acid segment” refers to a nucleic acid molecule that has been isolated free of total genomic DNA of a particular species. Therefore, a nucleic acid segment encoding a polypeptide refers to a nucleic acid segment that contains wild-type, polymorphic, or mutant polypeptide-coding sequences yet is isolated away from, or purified free from, total mammalian or human genomic DNA. Included within the term “nucleic acid segment” are a polypeptide or polypeptides, DNA segments smaller than a polypeptide, and recombinant vectors, such as, plasmids and other non-viral vectors.

The term “recombinant” may be used in conjunction with a polypeptide or the name of a specific polypeptide, and generally refers to a polypeptide produced from a nucleic acid molecule that has been manipulated in vitro or that is the replicated product of such a molecule. Recombinant vectors and isolated nucleic acid segments may variously include the PR1 or myeloperoxidase-coding regions themselves, coding regions bearing selected alterations or modifications in the basic coding region, or they may encode larger polypeptides that nevertheless include PR1 or myeloperoxidase-coding regions or may encode biologically functional equivalent proteins or peptides that have variant amino acids sequences.

A “nucleic acid” as used herein includes single-stranded and double-stranded molecules, as well as DNA, RNA, chemically modified nucleic acids and nucleic acid analogs. It is contemplated that a nucleic acid within the scope of the present invention may be of about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater nucleotide residues in length. Those of skill will recognize that in cases where the nucleic acid region encodes a tumor-associated HLA-restricted peptide, or polypeptide, the nucleic acid region can be quite long, depending upon the number of amino acids in the fusion protein.

It is contemplated that the HLA-restricted peptide, or polypeptide may be encoded by any nucleic acid sequence that encodes the appropriate amino acid sequence. The design and production of nucleic acids encoding a desired amino acid sequence is well known to those of skill in the art, using standardized codon tables (Table 3). In preferred embodiments, the codons selected for encoding each amino acid may be modified to optimize expression of the nucleic acid in the host cell of interest. The term “functionally equivalent codon” is used herein to refer to codons that encode the same amino acid, such as the six codons for arginine or serine, and also refers to codons that encode biologically equivalent amino acids. Codon preferences for various species of host cell are well known in the art. Codons preferred for use in humans, are well known to those of skill in the art (Wada et. al., 1990). Codon preferences for other organisms also are well known to those of skill in the art (Wada et al., 1990, included herein in its entirety by reference).

TABLE 3
Codon Table

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUG UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Prokaryote- and/or eukaryote-based systems can be used to produce nucleic acid sequences, or their cognate polypeptides, proteins and peptides. The present invention contemplates the use of such an expression system to produce the tumor-associated HLA-restricted peptide, or polypeptide. More specifically, the present invention employs the use of the insect cell/baculovirus system. The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the expression system disclosed in the invention, numerous expression systems exists which are commercially and widely available. One example of such a system is the STRATAGENE®'S COMPLETE CONTROL Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

A. Viral Vectors

There are a number of ways in which expression vectors may be introduced into cells. In certain embodiments of the invention, the expression vector comprises a virus or engineered vector derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986).

1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the vaccines of the present invention (Muzyczka, 1992). AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses have promise as gene delivery vectors in vaccines due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid (e.g., one encoding an antigen of interest) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

4. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

5. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

B. Nucleic Acid Delivery

Suitable methods for nucleic acid delivery to effect expression of compositions of the present invention are believed to include virtually any method by which a nucleic acid (e.g., DNA, including viral and nonviral vectors) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

### X. PHARMACEUTICAL VACCINE COMPOSITIONS, DELIVERY, AND TREATMENT REGIMENS

In an embodiment of the present invention, a method of treatment and prevention of autoimmune diseases or conditions; transplant rejection; or vasculitis, by the delivery of an HLA-restricted peptide, or polypeptide or expression construct is contemplated. Autoimmune diseases/conditions that may be treated or prevented include but are not limited to asculitis, Wegner's granulomatoses Addison's disease, alopecia, ankylosing spondylitis, antiphospholipid syndrome, Behcet's disease, celiac disease, chronic fatigue syndrome, Crohn's disease, ulcerative colitis, type I diabetes, fibromyalgia, autoimmune gastritis, Goodpasture syndrome, Graves' disease, idiopathic thrombocytopenic purpura (ITP), lupus, Meniere's multiple sclerosis, myasthenia gravis, pemphigus vulgaris, primary biliary cirrhosis, psoriasis, rheumatoid arthritis, rheumatic fever, sarcoidosis, scleroderma, vitiligo, vasculitis, small vessel vasculitis, hepatitis, primary biliary cirrhosis, rheumatoid arthritis, Chrohn's disease, ulcerative colitis, sarcoidosis, scleroderma, graft versus host disease (acute and chronic), aplastic anemia, myelodysplastic syndrome, cyclic neutropenia, T-LGL (T-large granular lymphocytic) leukemia.

An effective amount of the pharmaceutical vaccine composition, generally, is defined as that amount sufficient to detectably and repeatedly to ameliorate, reduce, minimize or limit the extent of the disease or condition or symptoms thereof. More rigorous definitions may apply, including elimination, eradication or cure of disease.

Preferably, patients will have adequate bone marrow function (defined as a peripheral absolute granulocyte count of >2,000/mm³ and a platelet count of 100,000/mm³), adequate liver function (bilirubin <1.5 mg/dl) and adequate renal function (creatinine <1.5 mg/dl).

A. HLA-Restricted Vaccine Administration

The routes of administration will vary, naturally, with the disease state, and include, e.g., intradermal, transdermal, parenteral, intravenous, intramuscular, intranasal, subcutaneous, percutaneous, intratracheal, intraperitoneal, intratumoral, perfusion, lavage, direct injection, and oral administration and formulation. Treatment regimens may vary as well, and often depend on the disease state and progression, and health and age of the patient. Obviously, certain types of disease will require more aggressive treatment, while at the same time, certain patients cannot tolerate more taxing protocols. The clinician will be best suited to make such decisions based on the known efficacy and toxicity (if any) of the therapeutic formulations.

A typical course -of treatment will involve multiple doses. Typical primary treatment involves a 6 dose application over a two-week period. The two-week regimen may be repeated one, two, three, four, five, six or more times. During a course of treatment, the need to complete the planned dosings may be re-evaluated.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of plaque forming units (pfu) for a viral construct. Unit doses range from 10³, 10⁴, 10⁵, 10⁶, 10⁷, 10⁸, 10⁹, 10¹⁰, 10¹¹, 10¹², 10¹³ pfu and higher. Alternatively, depending on the kind of virus and the titer attainable, one will deliver 1 to 100, 10 to 50, 100-1000, or up to about 1×10⁴, 1×10⁵, 1×10⁶, 1×10⁷, 1×10⁸, 1×10⁹, 1×10¹⁰, 1×10¹¹, 1×10¹¹, 1×10¹³, 1×10¹⁴, or 1×10¹⁵ or higher infectious viral particles (vp) to the patient or to the patient's cells.

B. Injectable Compositions and Formulations

One method for the delivery of a pharmaceutical according to the present invention is systemically. However, the pharmaceutical compositions disclosed herein may alternatively be administered parenterally, intravenously, intradermally, intramuscularly, transdermally or even intraperitoneally as described in U.S. Pat. No. 5,543,158; U.S. Pat. No. 5,641,515 and U.S. Pat. No. 5,399,363 (each specifically incorporated herein by reference in its entirety).

Injection of pharmaceuticals may be by syringe or any other method used for injection of a solution, as long as the agent can pass through the particular gauge of needle required for injection. A novel needleless injection system has been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermolysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

### XI. COMBINATION TREATMENTS

The compounds and methods of the present invention may be used in the context of autoimmune diseases/conditions. In order to increase the effectiveness of a treatment with the HLA-restricted compositions of the present invention, such as Pr3 or MYO peptide, polypeptide, protein, or expression construct coding therefor, it may be desirable to combine these compositions with other agents effective in the treatment of those diseases and conditions. For example, the treatment of autoimmune diseases or infections with therapeutic compounds of the present invention may be enhanced by combination with and other therapies.

Various combinations may be employed; for example, the HLA-restricted peptide is “A” and the secondary therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the therapeutic agents of the present invention to a patient will follow general protocols for the administration of that particular secondary therapy, taking into account the toxicity, if any, of the HLA-restricted peptide treatment. It is expected that the treatment cycles would be repeated as necessary.

A. Adjunct Autoimmune Therapy

Typical treatments for autoimmune diseases include various anti-inflammatories. For example, aminosalicylates, drugs that contain 5-aminosalicyclic acid (5-ASA), help control inflammation. Other 5-ASA agents such as olsalazine, mesalamine, and balsalazide, have a different carrier, offer fewer side effects, and may be used by people who cannot take sulfasalazine. Other non-steroidal anti-inflammatories include COX-selective and COX-preferential inhibitors.

Corticosteroids—such as prednisone and hydrocortisone—also reduce inflammation. They may be used by people who have moderate to severe ulcerative colitis or who do not respond to 5-ASA drugs. Corticosteroids, also known as steroids, can be given orally, intravenously, through an enema, or in a suppository, depending on the location of the inflammation. These drugs can cause side effects such as weight gain, acne, facial hair, hypertension, mood swings, and an increased risk of infection. For this reason, they are not recommended for long-term use.

Immunomodulators—such as azathioprine and 6-mercapto-purine (6-MP)—reduce inflammation by affecting the immune system. They are used for patients who have not responded to 5-ASAs or corticosteroids or who are dependent on corticosteroids. However, immunomodulators are slow-acting and may take up to 6 months before the full benefit is seen. Patients taking these drugs are monitored for complications including pancreatitis and hepatitis, a reduced white blood cell count, and an increased risk of infection. Cyclosporine A may be used with 6-MP or azathioprine to treat active, severe ulcerative colitis in people who do not respond to intravenous corticosteroids.

B. Adjunct Therapy for Infections

A varient of a biologicals are available to treat infectious diseases and include anti-bacterial (antibiotics, antiseptics), anti-virals, and anti-parastic agents. Any of these agents may be used in combination with the present invention.

### XII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Methodology

Patients and donors. Donors and patients were treated at the University of Texas M.D. Anderson Cancer Center on protocols approved by the Institutional Review Board. After informed consent, cells from the CML patients and their HLA-matched bone marrow (BM) transplantation donors were obtained. PBMCs from untreated CML patients or from patients receiving IFN were collected and cryopreserved for later analysis. Cells were separated using Ficoll-Hypaque gradient-density (Organon Teknika Corp., Durham, N.C., USA) and frozen in RPMI-1640 complete medium (CM) (25 mM HEPES buffer, 2 mM L-glutamine, 100 U/ml penicillin, 100 μg/ml streptomycin; Life Technologies Inc., Gaithersburg, Md., USA) supplemented with 20% heat-inactivated pooled human AB serum (AB; Sigma-Aldrich, St. Louis, Mo., USA) and 10% DMSO, according to standard protocols. Before use, cells were thawed, washed, and suspended in CM plus 10% AB. High-resolution HLA testing was performed by the HLA Laboratory at M.D. Anderson Cancer Center.

Peptide synthesis. PR1 (aa 169-177) peptide (VLQELNVTV (SEQ ID NO:1)) was synthesized by Bio-Synthesis (Lewisville, Tex., USA), and the HLA-A2-restricted CMV pp65 peptide (NLVPMVATV (SEQ ID NO:2)) was synthesized by the M.D. Anderson Protein and Nucleic Acid Facility, both to a minimum of 95% purity.

Cell lines and peptide binding. T2 cells (American Type Culture Collection, Rockville, Md., USA) were maintained in culture in CM plus 10% FBS (Atlanta Biologicals Inc., Norcross, Ga., USA). PR1 peptide was incubated at increasing concentrations for 18 hr at 37° C. with 100 μg/ml β₂m (Sigma-Aldrich) with 10⁶ T2 cells in 1 ml CM. Cells were washed twice with CM, stained with BB7.2 Ab, washed again, and then stained with FITC-labeled secondary Ab (Becton Dickinson Immunocytometry Systems, San Jose, Calif., USA). HLA-A*0201 expression was measured by FACS.

Peptide-specific CTLs were expanded in culture using methods described previously (Molldrem et al., 1996; Molldrem et al., 1997). Briefly, PBMCs from healthy donors or CML patients were stimulated in vitro with PR1, pp65, or flu peptides. T2 cells were washed three times in serum-free CM and incubated with peptide at the indicated peptide concentration for 2 hr in CM. The peptide-loaded T2 cells were then irradiated with 7,500 cGy, washed once, and suspended with freshly isolated PBMCs at a 1:1 to a 1:2 ratio in CM supplemented with 10% AB. After 7 days in culture, a second stimulation was performed, and the following day, 60 fU/ml of recombinant human interleukin-2 (rhIL-2) (BioSource International, Camarillo, Calif., USA) was added. After 14 days in culture, a third stimulation was performed, followed on day 15 by addition of rhIL-2. A fourth stimulation was performed on day 21, followed on day 22 by the addition of rhIL-2. After a total of 27-28 days in culture, the peptide-stimulated T cells were obtained and tested for peptide-specific and leukemia-specific cytotoxicity as well as for phenotypic analyses.

Tetramer synthesis. Production of MHC/peptide tetramers was described in detail elsewhere (Altman et al., 1996; Molldrem, 2000). Briefly, a 15-amino acid substrate peptide (BSP) for BirA-dependent biotinylation has been engineered onto the COOH terminus of HLA-A2. The A2-BSP fusion protein and human β₂m were expressed in Escherichia coli and were folded in vitro with the specific peptide ligand. The properly folded MHC-peptide complexes were extensively purified using FPLC and anion exchange and biotinylated on a single lysine within the BSP using the BirA enzyme (Avidity, Denver, Colo., USA). Tetramers were produced by mixing the biotinylated MHC-peptide complexes with phycoerythrin-conjugated (PE-conjugated) Neutravidin (Molecular Probes Inc., Eugene, Oreg., USA) at a molar ratio of 4:1. PR1 tetramers were validated by staining against a CTL line specific for PR1. CMV tetramers were validated by staining PBMCs from a CMV-immune individual.

Ab's and flow cytometry. For routine surface-antigen staining, 10⁶ PBMCs were incubated at 4° C. with Ab. After washing, cells were incubated with FITC-labeled CD8 (Caltag Laboratories Inc., Burlingame, Calif., USA) for 30 min on ice. Surface expression of TCR was determined with FITC-labeled TCR-αβ (PharMingen, San Diego, Calif., USA), and annexin V using FITC-labeled Ab (Caltag Laboratories). Proteinase 3 expression was determined with primary mouse Ab (Accurate Chemical & Scientific Corp., Westbury, N.Y., USA). Mouse monoclonal anti-HLA-A2.1 Ab BB7.2 and anti-HLA-ABC w6/32 were derived from culture supernatants of a hybridoma cell lines (American Tissue Culture Collection). Cells were washed and fixed in 2% paraformaldehyde and analyzed on a FACScan (Becton Dickinson Immunocytometry Systems), and data were analyzed using CELLQuest (Becton Dickinson Immunocytometry Systems) software. The minimum concentration of tetramer necessary to show distinctly different avidities was determined in titration experiments. Each tetramer reagent was titered individually and used at the optimal concentration. The tetramer concentration showing the maximal separation of fluorescence intensity of CTL populations was generally 10-20 μg/ml. Propidium iodide (PI) (Becton Dickinson Immunocytometry Systems) staining (1 μg/ml) was performed to exclude dead cells, according to the manufacturer's instructions. A “dump” channel was used with tetramer staining to eliminate monocytes and B lymphocytes with nonspecific binding to the HLA-A2 heavy chain by staining with PerCP-labeled CD14 and CD19 (both Becton Dickinson Immunocytometry Systems).

Detailed methodology for detection of t_{1/2} of tetramer dissociation was described elsewhere (Savage et al., 1999). Briefly, T cells were stained for 45 min with PR1/HLA-A2 tetramer, washed, and cooled to 4° C. To prevent rebinding of tetramer, cells were incubated in the presence of PI (1 μg/ml) with saturating amounts of BB7.2 Ab, and flow cytometry was used to measure fluorescence decay at 10-min intervals. A constant number of CD8⁺ events was acquired at each time point. Total fluorescence within the tetramer-positive gate was normalized per CD8⁺ cell, and the antigen-specific fluorescence was determined by subtracting the total fluorescence of control healthy donor lymphocytes from the observed total fluorescence of the lymphocyte populations following peptide stimulation. This antigen-specific fluorescence was normalized to the percentage of the total fluorescence at the initial time point and plotted on a logarithmic scale. Tetramer dissociation t_{1/2} was determined by plotting ln (normalized fluorescence) versus time, and is given by t_{1/2}=0.693/k, where k is the slope of the normalized fluorescence decay curve determined by the method of linear least squares.

CTL cytotoxicity assay. A semi-automated minicytotoxicity assay was used to determine specific lysis as described previously (Molldrem, 2000; Molldrem et al., 1996). Briefly, effector cells were prepared in doubling dilutions from 6×10³ to 25×10³ cells/well and were plated in 40 μl, 60-well Terasaki trays (Robbins Scientific, Sunnyvale, Calif., USA) with six replicates per dilution. Target cells (T2 cells, marrow-derived leukemic cells, or BM from a healthy donor) at a concentration of 2×10⁶ cells/ml were stained with 10 μg/ml of Calcein-AM (Molecular Probes Inc.) for 60 min at 37° C. After washing three times in CM plus 10% AB, target cells were resuspended at 10⁵ cells/ml, and 10³ target cells in 10 μl medium were added to each well containing effector cells. Wells with target cells alone and medium alone were used for maximum (max) and minimum (min.) fluorescence emission, respectively. After 4 hr incubation at 37° C. in 5% CO₂, 5 μl FluoroQuench (One Lambda Inc., Canoga Park, Calif., USA) was added to each well, and the trays were centrifuged for 1 min at 60 g before measurement of fluorescence using an automated CytoFluor II plate reader (PerSeptive Biosystems, Framingham, Mass., USA). The percentage of lysis was calculated as follows: % lysis={1−[(mean experimental mission−mean min.)/(mean max−mean min.)]}×100%.

### Example 2

### Clinical Trials

Based upon pre-clinical studies, the toxicity and efficacy of PR1 peptide vaccination for patients with refractory or relapsed myeloid leukemias was investigated. This study is being conducted in two phases: a Phase I initial toxicity phase (in order to determine initial vaccine safety), and a Phase II efficacy and toxicity phase. Nine patients will be studied in the Phase I portion, and up to 60 patients will be studied in the Phase II portion. Four patients have been enrolled thus far on Phase I. Details of the protocol are described below.

PR1 peptide is injected subcutaneously in incomplete Freund's adjuvant every 3 weeks for 3 injections to induce a PR1 specific host response against the myeloid leukemia. Both in Phase I and in Phase II, patients will also be evaluated for signs of immune reactivity. Before, during, and at the end of the 9 week period of vaccination, the peripheral blood mononuclear cells (PBMC) from the patients will be tested for the development of PR1 immune reactivity in vitro using cytotoxic T lymphocyte precursor frequency (CTLPf) assays against PR1-loaded target cells and against the patient's own leukemia (a measure of efficacy), PR1/HLA-A2 tetramer staining, 8-color flow cytometry for surface phenotype (memory/naive, activation), and cytolysis assays of bulk PR1-stimulated PBMC from the patients. The amount of Proteinase 3 expression in the leukemia cells is studied by cytoplasmic flow cytometry analysis. Any clinical responses (defined by standard criteria as hematological and/or cytogenetic responses) will be correlated with the in vitro testing. Vaccination at 3 dose levels of peptide with a fixed amount of IFA will be conducted, and stopping criteria will be guided by established toxicity criteria.

Phase I. In Phase I of this study, patients are assigned to three escalating peptide dose levels starting with dose level 1. Patients are followed in dose cohorts of size 3 for signs of dose limiting toxicity. Patients are assigned to the next highest dose level cohort only if no more than 1 of the 3 patients at any time at any dose level has ≧grade 3 non-hematological toxicity or autoimmune phenomena (i.e., dose limiting toxicity). At each dose level, the first patient entered must complete two of the three vaccinations prior to initiation of the second patient in that cohort at that dose level. Before the third patient is enrolled, the first patient in each dose cohort must complete all vaccinations and the second patient must complete at least two vaccinations prior to initiation of vaccination in the third patient. Again, if <grade 3 non-hematological toxicity and no autoimmune phenomena are observed in the first three patients, then the next dose level will accrue patients in a similar manner. If more than ⅓ of the patients enrolled at any time at any dose level have grade III or IV non-hematological toxicity, then maximal tolerated dose (MTD) has been exceeded and this and all higher peptide dose levels will be eliminated from Phase II of the study. Dose escalation to the next dose level will occur only after the third patient has been followed for three weeks after the third vaccination in the series and has no dose limiting toxicity.

If an allergic reaction ≧grade 2 occurs in any patient, no further vaccinations will be given in that patient and they will be taken off study. Allergic reactions will be treated with solumedrol 1 mg/kg IV bolus, benadryl 50 mg IV bolus, and epinephrine 0.5 mg S.C.

Phase II. Phase II of the study will be conducted according to a continuous reassessment statistical model. Up to 60 patients will be randomized among three dose levels, with a maximum of 20 patients per dose level. Only those dose levels without dose limiting toxicity as determined in Phase I of this study will be examined in Phase II. Both toxicity and efficacy will be determined as primary endpoints. Patients will be monitored in cohorts of size 4, and all patients in any cohort will be observed for at least 2 weeks from the first dose of the last patient in the group in the absence of grade III or IV non-hematological toxicity before continuing to the next cohort. Toxicity information will be carefully accrued using established. If grade III or IV non-hematological toxicity is observed during the 9 week study period in 2 of the first 4 patients (the first cohort), 3 of the first 8, 4 of the first 12, or 5 of the first 16 in any of the three dose groups, then that dose level will be terminated and patients will be treated only on the remaining dose levels. Moving to the next cohort will occur only if dose-limiting toxicity is not reached in the number of patients defined above for each cohort.

There will be a two-week observation period before the next vaccination will be given in any patient that experiences grade II non-hematological toxicity <dose limiting toxicity. If the toxicity decreases to grade I or less within those two weeks, the patient will be given the next vaccination. If the grade II toxicity does not decrease to grade I or less within two weeks, the patient will be taken off protocol (removed from study).

Any grade toxicity that provides clear evidence of an autoimmune reaction will be considered a dose limiting toxicity. Such a reaction will preclude further administration of peptide under this protocol, and the study will be terminated. If there is evidence that vaccine administration has produced a Wegener's-like vasculitis or inflammatory disease, then the study will be terminated.

Efficacy, defined as an immune response to PR1 vaccine, will also be determined as a primary endpoint of the study. If none of the first 12 patients have an immune response at a particular dose level, then that dose level will be terminated. Patients will not be retreated in this protocol after the required 3 immunizations, nor will the dose be escalated beyond 1.0 mg of PR1 peptide.

The maximal tolerated dose (MTD) is defined as the highest peptide dose that does not cause dose-limiting non-hematological toxicity beyond the allowable number of patients stated for each phase at each dose level cohort (dose limiting toxicity). The MTD will be determined in either Phase I or Phase II of this study if: (1) dose-limiting toxicity is reached in more than 1 patient of 3 at each dose level cohort in Phase I, or (2) if dose-limiting non-hematological toxicity is exceeded in 3 of the first 8, 4 of the first 12, or 5 of the first 16 in any of the three dose groups in Phase II.

### Example 3

### Generation of PR1-CTL and Ex Vivo Studies

Experimental and clinical evidence indicates that lymphocytes from HLA-matched allogeneic normal donors exert a powerful graft-versus-leukemia (GVL) effect when used to treat patients with myeloid leukemia (Drobyski et al., 1994; Horowitz et al., 1996). Donor lymphocyte infusions (DLI) alone, when administered to patients that have relapsed after allogeneic bone marrow transplantation (BMT) can cure patients with myeloid leukemia (Collins et al., 1997; Kolb and Holler, 1997). However, graft-versus-host disease (GVHD) is an unwanted and sometimes lethal complication of DLI treatment that occurs in nearly 50% of patients. The GVL and GVHD target antigens of these lymphocytes are largely unknown. GVL may be enhanced and GVHD eliminated after DLI if (1) antigens that were the favored targets of GVL are known and (2) an efficient ex vivo process for enrichment of GVL-causing lymphocytes based on antigen specificity is developed.

In this study, it was determined whether high and low affinity PR1-specific CTL can be expanded from normal donors ex vivo and then enriched using antigen-coated microbeads for transfer to myeloid leukemia patients, in place of conventional DLI, in order to deliver GVL without GVHD. Methods to more efficiently generate PR1-CTL, enrich high affinity PR1-CTL using microbeads, and expand selected PR1-CTL ex vivo for adoptive immunotherapy were also investigated.

Methodology. PR1 peptide was combined into the binding region of the HLA-A2 heavy chain, and the resulting protein folded with P₂-microglobulin and attached the resulting PR1/HLA-A2 monomer onto 50 nm magnetic microbeads (Miltenyi Co.) (Wang et al., 2000). To do this, the technology used was adapted to assemble PR1/HLA-A2 tetramers, where heavy chain monomers are biotinylated at the C terminus and combined in a 4:1 molar ratio with streptavidin, which has in turn been conjugated to phycoerythrin (PE). The PR1 monomer-conjugated microbeads can then be passed through a sterile column that is surrounded by a magnet that traps microbead-adherent T cells. After the non-adherent cells pass through the column, the column is removed from the magnet and the PR1-specific T cells attached to the microbeads can be collected. This method allows for the selection of PR1-specific T cells, which could be further expanded and given to patients with myeloid leukemia to facilitate GVL without GVHD.

Determining the most efficient in vitro method for short-term expansion of PR1 antigen-specific T lymphocytes. The results show that PR1-specific cytotoxic T lymphocytes (CTL) are present at frequencies from 1/15,000 to 1/300,000 in normal donors. Therefore, the PR1-specific CTL will first be expanded so enough cells are available for subsequent enrichment by magnetic bead separation. The efficiency of antigen presenting cells (APC) produced by different methods to expand short-term polyclonal PR1-specific CTL will be compared. Peripheral blood mononuclear cells (PBMC), as APC, will be compared to dendritic cells (DC) derived from peripheral blood monocytes after a 2-hr adherence step. One week-old DC will be derived after incubating adherent cells from the same healthy HLA-A2+ donors with either 10 ng/ml IL-4+1000 U/ml GM-CSF or 1000 U/ml IFN-α+1000 U/ml GM-CSF (Verdijk et al., 1999; Santini et al., 2000). The resulting APC will be pulsed weekly with PR1 at 10 μg/ml plus IL-2 at 20 U/ml and combined with responder PBMC in ratios from 1:1 to 1:20. These APC will be compared to standard T2 cells, a cell line unable to present endogenous antigens due to TAP ½ deficiency and can only present peptides pulsed onto the cell surface (Salter and Cresswell, 1986). Preliminary studies using T2 cells as peptide-pulsed APCs expanded the PR1-specific CTL to between 2% to 8% of the total lymphocyte culture by 28 days (Molldrem et al., 2000).

In addition, the APC methodology described above will also be compared to the results obtained from pulsing Drosophila melanogaster S2 cells with PR1 peptide (Janetzki et al., 2000). S2 cells have been transfected with the HLA-A2.1 heavy chain, β-2M, ICAM-1, and the co-stimulatory molecule B7.1. By pulsing peptides on the surface of transfected S2, these cells have demonstrated the ability to act as efficient APC during the critical first round of peptide antigen stimulation of CTL in vitro. The advantages of using S2 cells as an APC population is that they can be propagated at room temperature and subsequently require low maintenance, a critical element for eventual clinical application.

After 3 to 4 weeks in culture, the resulting PR1-specific CTL will be quantified and characterized using the PR1/HLA-A2 tetramer in combination with antibodies to CD8, CD69, HLA-DR, CD25, LFA-1, CD54, CD2, intracellular y-interferon, and CD45(RO) conjugated to different fluorochromes (Molldrem et al., 1999; Molldrem et aL., 2000). Cells will subsequently be analyzed using a MoFlo cytometer capable of simultaneous 10-color cytofluorometric analysis. Efficiency of the various APC conditions can be directly compared by quantifying PR1-specific CTL using the tetramer and the other markers will allow comparison of activation state, functional state and memory phenotype.

To determine whether high and low affinity antigen-specific cytotoxic T lymphocytes can be separated and enriched using PR1/HLA-A2 monomer-coated microbeads. From the preliminary studies, it has been shown that by using low doses of tetramer reagent a discrimination can be made between CTL with high and low affinity T cell receptor (TCR) based on high and low tetramer staining as assessed by FACS (Molldrem et al., 1998). The T_{1/2} of tetramer binding has been shown to correlate with staining intensity, and specific lysis of CML target cells correlates with TCR affinity. High affinity PR1-CTL were elicited from normal donors in the presence of low-dose PR1 (2 μg/ml) and low affinity PR1-CTL were elicited using high-dose PR1 (200 μg/ml) (Molldrem et al., 1998).

To preferentially select PR1 antigen-specific T-cells, streptavidin-coated microbeads (from Miltenyi, Inc.) will be used. Beads will be conjugated with PR1 plus HLA-A2 through the biotin-labeled C-terminus of the heavy chain (Wang et al., 2000). PR1-pulsed short-term CTL lines will be incubated for 30 min with PR1/HLA-A2 coated microbeads and selected using the MACS™ magnetic column. Bead-selected and non-selected CTL will be quantified by flow cytometry using the PR1/HLA-A2 tetramer and compared to the starting polyclonal CTL lines. It is estimated that yields will range from 85% to 100% and PR1-specific CTL purity will range from 11% to 21% after the selection process (i.e. a 3- to 5-fold increase in purity of antigen-specific CTL), based on preliminary studies using PR1-pulsed T2 cells to elicit PR1-specific CTL (Molldrem et al., 1999; Wang et al., 2000).

The selected PR1-specific CTL will be tested for functional activity. Specific lysis of PR1 -coated T2 cells, CML and AML marrow cells, and normal HLA-A2 marrow will be assessed using the PR1-specific CTL and compared to lysis by the non-selected CTL. The method to select the highest affinity PR1-specific CTL will be optimized using different ratios of PR1/HLA-A2 monomers, microbeads, and starting cell populations to achieve the highest yield and greatest purity of high affinity PR1-specific CTL.

To determine whether lymphocytes, separated using PR1/HLA-A2 monomer-coated microbeads, can be expanded to a sufficient number for use in adoptive immunotherapy of myeloid leukemia. It is estimated that from 1 to 5×10⁶ CTL may be needed to treat patients with relapsed leukemia after BMT (Mackinnon et aL., 1995). By optimizing a CTL expansion technique in combination with the bead enrichment, the requirement of a leukapheresis product may be reduced to as little as 100 ml of peripheral blood. To achieve this goal, it will be determined whether the bead-enriched PR1-specific CTL can be further expanded in short term culture. Two methods will be directly compared: the optimized technique, as determined previously, will be used to expand PR1-CTL by weekly restimulation with PR1-coated APCs, versus a non-specific method. Anti-CD3+ anti-CD28 coated Dynal beads will be coincubated with the bead-enriched PR1-specific CTL in culture media containing varying doses of IL-2 (Garlie et al., 1999; Levine et al., 1998). In controlled experiments, other cytokines such as IL-12 and interferon will be supplemented in order to optimize the number of PR1-CTL (Fallarino et al., 1996). PR1-specificity will be confirmed at the end of culture using PR1/HLA-A2 tetramers and CTL functional activity will be ascertained using standard cytotoxicity experiments against PR1-coated T2 cells and HLA-A2+ leukemia cell targets.

If non-specific expansion of CTL (with anti-CD3 and anti-CD28) is found to be efficient and can maintain PR1-specific CTLs, then this method will also be tested PR1 or to bead selection and compared to the PR1-specific expansion method described above. This may eliminate the requirement for further CTL expansion after bead enrichment.

These methods, once optimized, can be easily transferred to the clinical setting to treat patients with myeloid leukemia using a Miltenyi MACS™ magnetic bead system to select CD34⁺ progenitor cells from marrow donors for clinical use. Furthermore, these methods may be used to select CTL with different specificities for other tumor antigens, minor antigens, and viral antigens. Such CTL may be elicited and selected in a single step and adoptively transferred using the methods described herein.

Based on the techniques described herein efficient clinical scale-up procedures will be determined for adoptive transfer of antigen-specific T lymphocytes.

### Example 4

### Preliminary Studies

Peptide selection and binding assays. In the first step to generating T cells which could be used for adoptive immunotherapy of myeloid leukemias, several peptides derived from the published sequence of Pr3 which were predicted to bind to HLA-A*0201 using a published algorithm (Parker et al., 1994) were identified. This allele was chosen because its high frequency in the US population (49% of individuals) would maximize the therapeutic relevance of any eventual immunotherapeutic strategy. Of the 9 peptides predicted to have sufficiently high binding affinities based on the known HLA-A2.1 binding motif, the two with the highest predicted binding were subsequently synthesized (designated PR1 and PR2) (Table 4). The peptides were synthesized by Biosynthesis (Lewisville, Tex.) to a minimum of 95% purity

TABLE 4

Predicted Binding-Half Life of
HLA-A*0201-Restricted Pr3 Peptides

| | | | |
|---|---|---|---|
| | | SEQ | Predicted |
| Peptide | Peptide | ID | Binding Half-life |
| Abbreviation | Sequence | NO: | (minutes) |

| | | | |
|---|---|---|---|
| PR1 | VLQELNVTV | 1 | 304 |
| PR2 | RLFPDFFTRV | 3 | 1169 |
| PR3 | VLQELNVTVV | 4 | 215 |
| PR4 | NLSASVTSV | 5 | 160 |
| PR5 | IIQGIDSFV | 6 | 83 |
| PR6 | VLLALLLISGA | 7 | 66 |
| PR71 | KLNDILLIQL | 8 | 221 |
| PR7V | KLNDVLLIQL | 9 | 220 |
| PR8 | QLPQQDQPV | 10 | 66 |
| PR9 | FLNNYDAENKL | 11 | 53 |

Peptide binding to HLA-A2.1 was confirmed using two assays. In the first, indirect flow cytometry was used to measure HLA-A2.1 surface expression on the A2+T2 cell line coated with the peptide. T2 cells are a human lymphocyte line that lacks TAP1 and TAP2 genes and therefore cannot present endogenous MHC class I restricted antigens. If the peptide effectively bound HLA-A2.1, it stabilized the complex with (β2-microglobulin and increased HLA-A2.1 surface expression, which could be measured using flow cytometry. An HLA-A2.1 specific monoclonal antibody (BB7.2, ATCC, Rockville, Md.) followed by a FITC-labeled secondary antibody (CalTag Laboratories, Burlingame, Calif.) was used to measure surface expression of HLA-A2.1.

In the second assay, the dissociation rate of I¹²⁵-labeled (β2-microglobulin from the heterotrimer complex of the HLA-A2.1 heavy chain, peptide, and β2-microglobulin was measured, which allowed calculation of binding half-life (T_{1/2}). The labeled heterotrimer complex was separated from unincorporated (β2-microglobulin by high-performance liquid chromatography gel filtration, and the half-time of dissociation of β2-microglobulin was determined by subjecting aliquots of the complex to a second round of gel filtration.

Both PR1 and PR2 showed increased surface HLA-A2.1 expression compared with T2 cells with no added peptide, as the background for HLA-A2.1 expression, (Table 5). The control influenza peptide is an Influenza B nucleoprotein (aa 85-94; Flu), and Tax is an HTLV-1 peptide (aa 11-19), both with known high binding affinity to HLA-A2.1. The long measured TU2 as measured using (β2-microglobulin dissociation confirmed the binding of PR1 and PR2 to HLA-A2.1.

TABLE 5
Measured Binding of PR1 and PR2 to HLA-A2.1

| | Median Channel of | |
|---|---|---|
| | Fluorescence of HLA-A2 | |
| Peptide | Expression on T2 | T_{1/2} (minutes) |
| PR1 | 294 | 1460 |
| PR2 | 273 | 140 |
| HTLV-1 Tax | — | 8,000 |
| Influenza nucleoprotein | 194 | — |
| Background HLA-A2 | 22 | — |

Induction of Primary CTL responses to peptides. These peptides were next used to stimulate T cells specific for peptide-coated targets. PBMC from a normal healthy donor heterozygous for HL-A2.1 were stimulated with peptide-pulsed T2 cells. The T2 cell line has been used by others as an antigen presenting cell for the generation of peptide-specific CTL. Briefly, T2 cells (which co-express the costimulatory molecule B7.1) were washed 3 times in serum-free RPMI culture media supplemented with penicillin/streptomycin and glutamine (CM) and incubated with peptide at 20 μg/mL for 2 hr in CM. The peptide loaded T2 cells were then irradiated with 7500 cGy, washed once, and suspended with freshly isolated PBMC at a 1:1 ratio in CM supplemented with 10% human serum (HS) (Sigma, St. Louis, Mo.). After 7 days in culture, a second stimulation was performed and the following day, 60 IU/mL of recombinant human interleukin-2 (rhIL2) (Biosource International, Camarillo, Calif.) was added. After 14 days in culture a third stimulation was performed, followed on day 15 by addition of rhIL-2. A fourth stimulation was performed on day 21 followed on day 22 by the addition of rhIL2. After a total of 27 days in culture, the peptide-stimulated T cells were harvested and tested for peptide-specific cytotoxicity toward Calcein AM-labeled T2 cells, leukemia cell lines, and fresh human leukemia cells.

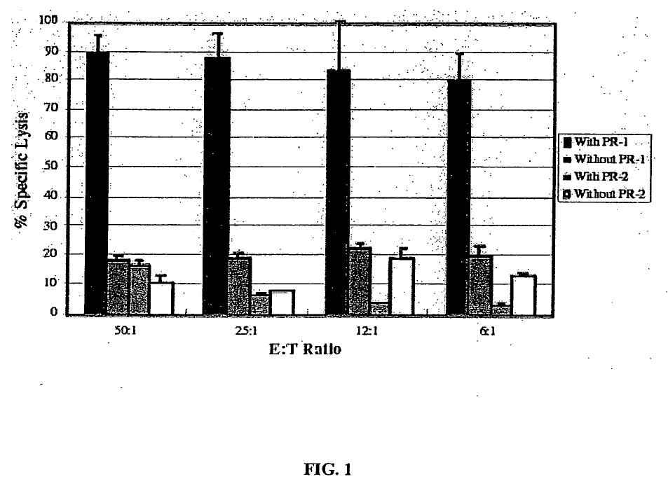

FIG. 1 shows the peptide-specific lysis of the CTL lines against T2 cells loaded with either 1.0 μg/mL of PR1 or PR-2, or T2 cells without added peptide, at varying effector to target (E:T) ratios. The CTL line generated against the PR1 peptide demonstrated high specific lysis against PR1-loaded target cells, whereas the CTL line generated against PR-2 did not demonstrate any significant cytotoxicity (Molldrem et al., 1996). Cytotoxicity toward T2 cells loaded with HTLV-1 tax (aa 11-19), an irrelevant peptide with high binding affinity to HLA-A2.1, was also measured (data not shown) and resulted in <20% specific lysis at E:T ratios of 50:1 by CTL specific for either PR1 or PR-2. CTL response toward PR1 was shown to be specific for target cells expressing the HLA-A2.1 molecule. This and the cytotoxicity observed was HLA-A2.1-restricted.

PR1 specific CTL preferentially lyse human myeloid leukemia cells. It was next determined whether the PR1-specific CTL line was capable of lysing allogeneic human myeloid leukemia cells from HLA-A2.1 positive individuals. Table 6 lists the HLA type and leukemia type of target cells used. As controls, two cell lines expressing low levels of Pr3 were used: HLA-A2.1 transfected K562 cells and U937 cell line which lack HLA-A2.1 and would therefore be incapable of presenting peptides in an HLA-A2.1-restricted manner. Cryopreserved bone marrow cells from patients P1-P4, and marrow cells from a healthy normal volunteer (D2, a bone marrow donor for an allogeneic bone marrow transplant performed on patient P3) were thawed and used as targets for the PR1-specific CTL line.

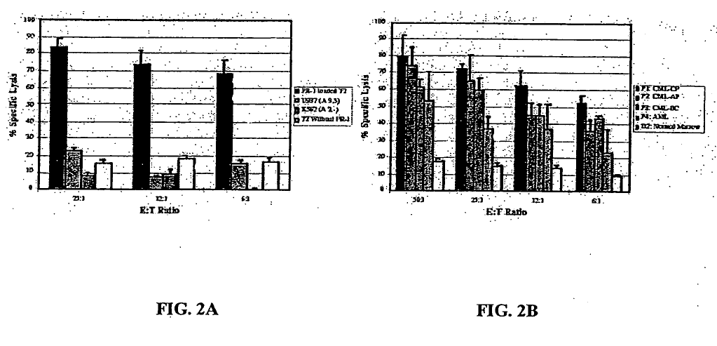

FIG. 2 shows the combined results of three separate experiments from three PR1-specific CTL lines. In FIG. 2A, the specific lysis by PR1-specific CTL, at various E:T ratios, of either U937 cells, HLA-A2.1-transfected K562 cells, or T2 cells with or without exogenously added PR1 peptide at 1.0 μg/mL is shown. The specific lysis of U937 and HLA-A2.1-positive K562 cells by PR1-specific CTL was lower than the background lysis observed against T2 cells without added peptide (Molldrem et al., 1996).

FIG. 2B demonstrates the cytotoxicity of the CTL line against HLA-A2.1-positive human myeloid leukemia cells. Marrow cells from three patients with CML (P1, P2, and P3) as well as one patient with AML M4 (P4) were readily lysed, with 53% specific lysis against P1 (a patient with CML in chronic phase) at an E:T ratio of only 6:1 (Molldrem et al., 1996). Marrow cells taken from a normal healthy donor (D2) demonstrate only background lysis (<20% lysis), similar to that of the control T2 cells without added PR1 peptide.

TABLE 6
Patient and Donor Cell HLA Types Used For Cytotoxicity Experiments

| Patient/Donor | Cell Description | HLA-A | HLA-B | HLA-DR |
|---|---|---|---|---|
| D1 | Normal PBMC | 1, 2 | 8, 27 | 3, 16 |
| D2 | Normal Marrow | 2, 26 | 13, 58 | 7, 7 |
| | U937 Cell Line | 3, 3 | 18, 51 | 14, 16 |
| P1 | CML-CP | 2, 28 | 35, 51 | 9, 14 |
| P2 | CML-AP | 1, 2 | 63, 63 | 8, 13 |
| P3 | CML-BC | 2, 26 | 13, 58 | 7, 7 |
| P4 | AML | 2, 3 | 35, 52 | 4, 13 |
| P5 | MDS (RAEB) | 2, 24 | 27, 62 | 1, 13 |

Abbreviations:
PBMC = peripheral blood monouclear cells;
CML-CP = chronic myelogenous leukemia, chronic phase;
CML-AP = chronic myelogenous leukemia, accelerated phase;
CML-BC = chronic myelogenous leukemia, blast crisis;
MDS (RAEB - myelodysplastic syndrome, refractory anemia with excess of blasts.

In previous studies, Pr1 specific CTL was found to preferentially inhibit colony forming units in leukemia patient samples, but not in normal donor samples (Molldrem et al., 19997). Cell contact was found to be required for inhibition of CFU-GM, and inhibitory factors elaborated into the supernatant by CTL1 effector cells were not responsible for colony inhibition (Molldrem et al., 1997).

Inhibition of CML CFU-GM correlates with Pr3 overexpression in target cells. Target cells were examined for cytoplasmic Pr3 expression by flow cytometry. After permeabilizing the cell membrane, indirect staining was performed using an antibody to Pr3 and a second FITC-labeled antibody, followed by flow cytometry. Table 7 lists the perentage of cells in the sample population that stain positive for Pr3 and the median fluorescence intensity of intracellular Pr3 staining. Median fluorescence intensity of leukemia samples was nearly five times the median fluorescence intensity of normal samples (1399 vs 298, p=0.009).

TABLE 7
Proteinase 3 Expression in Normal (D1-D5) and Leukemia (P1-P5)
Marrow Target Cells Used in Colony Inhibition Studies

| | | Median Channel |
|---|---|---|
| Patient/Donor | % Positive Cells | Fluorescence |
| CTL1 | 0 | 0 |
| CTL2 | 0 | 0 |
| D1 | 73.4 | 288 |
| D2 | 80.8 | 267 |
| D3 | 77.6 | 337 |
| D4 | 84.5 | 301 |
| D5 | 64.6 | 247 |
| P1 | 89 | 1011 |
| P2 | 98.6 | 1866 |
| P3 | 66.2 | 1437 |
| P4 | 94.7 | 1281 |
| P5 | 87.6 | 1641 |

Pr3 is highly expressed in leukemia but not in normal CD34 cells. To confirm that Pr3 was expressed in early CD34 positive CML cells, marrow was collected from patients. Marrow was obtained from a patient with CML in BC (P3), and normal CD34 cells for comparison were obtained from G-CSF mobilized peripheral blood mononuclear cells from a normal donor (D3). Cells were first labeled with PE conjugated anti-CD34 antibody (Becton Dickinson, San Jose, Calif.), followed by cytoplasmic indirect staining for Pr3. The inventors have shown that CML blasts that were CD34 positive and highly expressed Pr3 as compared to CD34 negative blasts and normal CD34 positive cells. This shows that very early progenitor cells overexpress Pr3 whereas there is very little Pr3 expression in a small number of normal progenitor cells.

Expression of proteinase 3 following transfection into and HMy2.CIR and HMy2.CIR-A2 cells. To provide further evidence that the PR1 peptide was processed and presented on the surface of a cell, a human B cell line (that does not express Pr3) was transfected with Pr3 and tested whether PR1 specific CTL could lyse the transfected cells. The parental HMy2.CIR cell line, which has lost expression of HLA-A and -B, and the HLA-A2.1 -transfected HMy2.CIR cells (named HMy2.CIR-A2), were both transfected with Pr3 as described below.

The previously published DNA sequence encoding Pr3 was used to design primers for cloning the cDNA from a normal bone marrow sample (Sturrick et al., 1992). RNA was extracted from normal donor bone marrow cells using the RNA STAT solution (Teltest). One microgram of RNA was reversed transcribed into cDNA using a RT PCR kit (Perkin-Elmer, Norwalk, Conn.). Half of the iotal cDNA was then amplified using the primers Pr3C-F 5′-CTGGACCCCACCATGGCTCA-3′ (SEQ ID NO:12) which included the ATG start codon, and Pr3C-R 5′-CGCCACAGTGTTCGGGGAAG-3′ (SEQ ID NO:13) and a high-fidelity polymerase (Takara, Madison, Wisc.) according to the manufacturer instructions. The PCR product was cloned into the pCR2 vector using the TA cloning kit (Invitrogen, Carlsbad, Calif.) and then subcloned into the mammalian expression vector pCDNA3.1 (Invitrogen) containing the CMV promoter and the Zeocin resistance marker. The resulting plasmid DNA was named pRTZ.2.

Ten million HMy2.CIR (ATCC, Rockville, Md.), or 10 million HMy2.CIR-A2 cells were each washed in phosphate buffered saline (PBS) and re-suspended in 0.8 mL of ice-cold PBS and put into an electroporation cuvette with 20 μg of circular plasmid DNA. The mixture was exposed to an electric pulse of 320 volts at 960 microF and incubated on ice for 10 min. Cells were then added to 10 mL of pre-warmed CM. The following day, the CM was replaced. On day 2, cells were grown under selection conditions using 200 μg/mL Zeocin. Following one week of selection, the cells were subcloned by limiting dilution under Zeocin selection, and 6 clones were isolated.

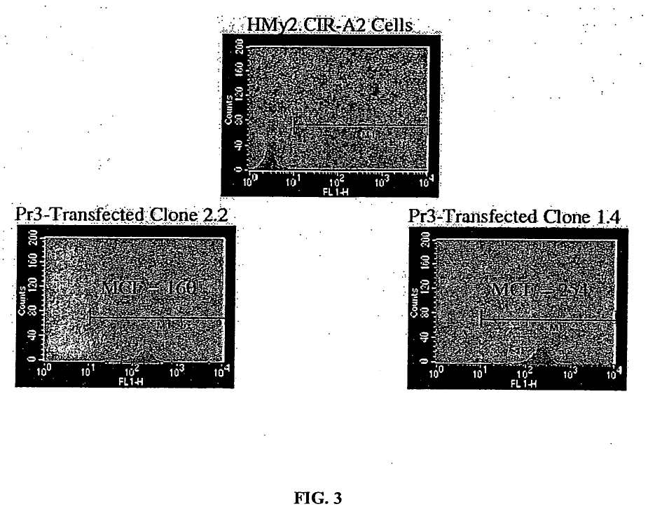

The human B cell line HMy2.CIR-A2 transfected either with the Pr3-containing vector pRTZ.2, or the empty vector containing the reporter gene, CAT, was tested for intracellular Pr3 expression by flow cytometry analysis of as previously described. Two clones with the highest expression are shown in FIG. 3.

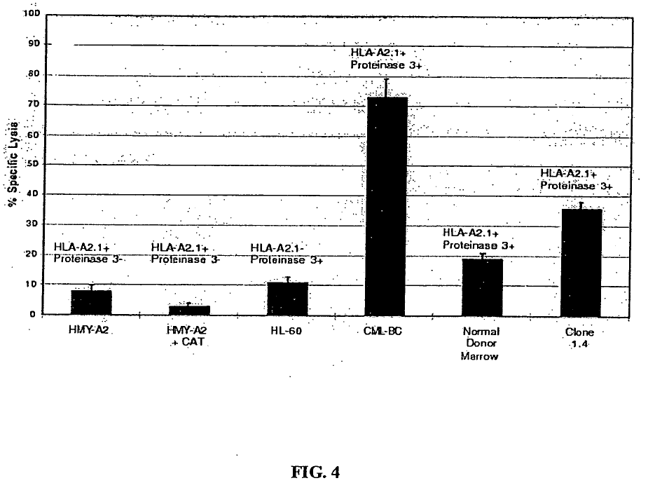

HMy.2.CIR-A2 cells transfected with Pr3 are susceptible to lysis by PR1 specific CTL. It was next determined whether the transfected HMy2.CIR-A2 cells could be lysed by PR1 specific CTL. The PR1 specific CTL were elicited in the manner previously described, and after 22 days in culture were combined at a 25:1 E:T ratio with HMy2.CIR-A2 transfected either with the Pr3-containing vector pRTZ.2, or the empty vector containing the reporter gene, CAT. PR1 specific CTL were also combined with control marrow cells from a CML patient in blast crisis (P3), HL-60 cells (which express Pr3, but lack HLA-A2.1), and marrow cells from a normal donor (D3). PR1 specific CTL demonstrated 36% specific lysis of the Pr3-transfected cells (clone 1.4), but only background lysis of normal marrow cells, HL-60 cells, HMy2.CIR cells transfected with the empty vector (CAT), and the non-transfected parent B cell line HMy2.CIR (HMY A2; FIG. 4). These results suggest that the PR1 peptide is processed and presented on the surface of the Pr3 transfected HMy2.CIR cells. Although the level of Pr3 expression was lower than that found in leukemia cells, the B cell line may be more efficient at processing and presentation, thus compensating for the lower level of Pr3 expression.

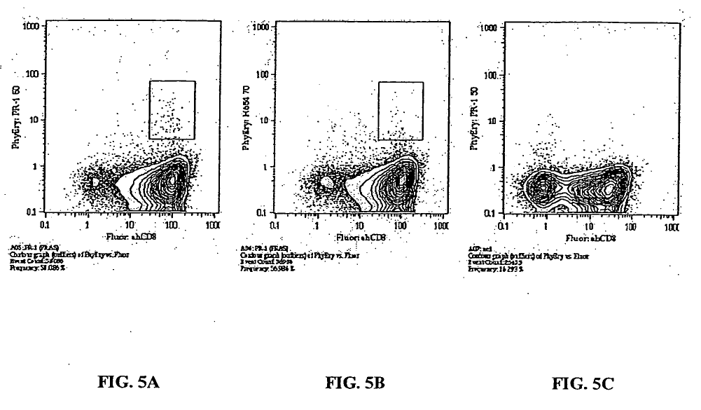

PR1-specific CTL identified in PBMC from normal donors by limiting dilution analysis and by using a PR1-HLA-A2 heavy chain tetramer. In order to determine the frequency of PR1-specific CTL in the PBMC of 2 HLA-A2+normal donors (donors 2 and 3) and one patient with chronic phase CML (donor i), a modified limiting dilution assay (LDA) designed for detecting low frequency lymphocyte precursors (CTLP) using a limited amount of patient material was used. This assay was used to determine the CTLP frequency (CTLPf) on day 0 and again after 20 days of PR1-stimulated expansion in bulk culture. The details of this assay are described herein. These results were compared to the results of a separate assay, using a reagent where CTL with PR1-specificity can be analyzed by flow cytometry. In this assay, the HLA-A2.1 heavy chain (modified to contain a biotin binding site near the C-terminal end of the A2 heavy chain) plus (β2-microglobulin is combined with the PR1 peptide, which is then combined with streptavidin conjugated to phycoerythrin (PE) (Altman et al., 1996). This “PR1-tetramer” was therefore used to directly label CTL with TCR specific for HLA-A2.1-bound PR1. FIG. 5 shows that the PR1-tetramer can be used to identify a distinct population of CTL with specificity for HLA-A2.1-bound PR1 amongst a 17 day-old bulk culture of PR1-stimulated PBMC.

Selection of a purified population of PR1-specific CTL from bulk culture is possible using a PR1-HLA-A2 heavy chain tetramer. In order to determine whether a purified population of PR1-specific CTL could be obtained from bulk culture CTL stimulated with PR1, the PR1-tetramer to FACS sort for CTL that expressed PR1-specific TCR was used. In this way, an enriched population of CTL might quickly be obtained without the usual requirement of cloning by limiting dilution followed by expansion, a process that may take several weeks to months.

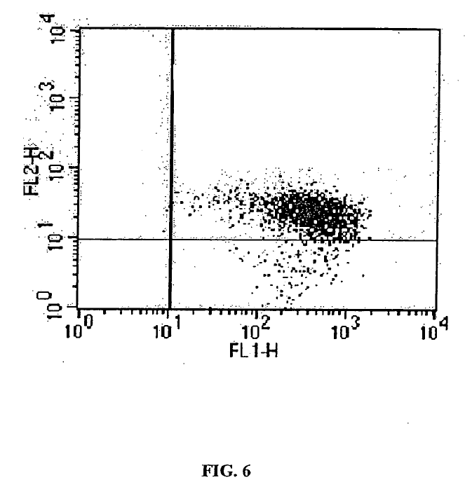

A 32 day-old PR1-stimulated CTL culture was obtained from a starting population of PBMC from an HLA-A2.1+normal donor using the methods previously described. When the cells were dual-labeled with anti-CD8 and the PR1-tetramer, 4.3% of the bulk culture CTL were double-positive. An aliquot of 5×10⁶ CTL was washed three times with PBS and labeled with anti-CD8 conjugated to FITC (Caltag Laboratories, Burlingame, Calif.) for 2 hr at 4° C. The cells were then washed three times with PBS and labeled with the PE-conjugated PR1-tetramer for 2 hr at 4° C. This was followed by a third labeling with both anti-CD4 and PI which was used as a dump during sorting. The cells were then sorted for dual-staining running CellQuest and CloneCyt software. 1.1×10⁵ CTL were recovered with >95% viability (a yield of 2.2% of the total population, or 55% of the PR1-specific population) and the sorted population is shown in FIG. 6.

The sorted CTL were then placed back into culture at 1×10⁶ cells/mL of CM supplemented with 10% human serum (Sigma) and 100 IU/mL IL-2 (Biosource International). After 24 hr, the cells were collected and tested for the ability to lyse fresh leukemia target cells. The PR1-sorted CTL were then compared to the non-sorted bulk culture CTL in a 4-hr cytotoxicity assay. Effector cells were washed three times with PBS and co-incubated at E:T ratios of 20:1 to 2.5:1 with target bone marrow cells from an HLA-A2.1+ patient with accelerated phase CML and with bone marrow cells from an HLA-matched normal donor for 4 hr. Target cells (1000 cells/well) were labeled with Calcein AM for 90 min prior to co-incubation, as in previous experiments.

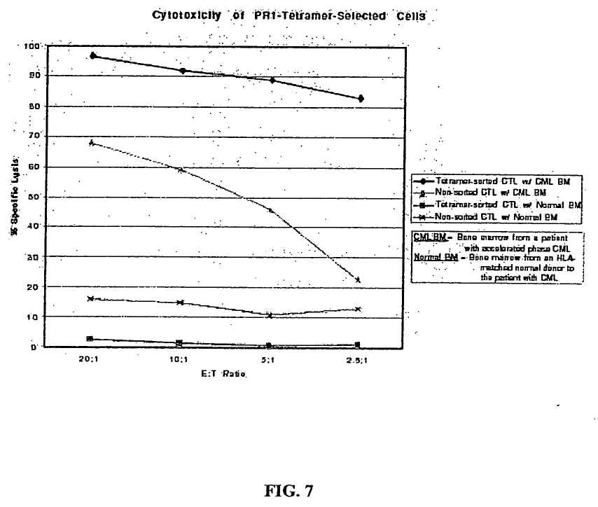

FIG. 7 shows that the PR1-sorted CTL produced significantly greater specific lysis of CML marrow than non-sorted CTL at all E:T ratios, and that background lysis of normal marrow was reduced to near zero. The CTL population depleted of PR1-specific CTL after FACS sorting was also examined for specific lysis of the CML marrow and no specific lysis was found at any E:T ratio. These results demonstrate that the PR1-tetramer can be used to select a homogeneous CTL population with a higher degree of specificity toward leukemia than bulk culture PR1-stimulated CTL. Other investigators studying melanoma antigens have obtained similar results. Using a MelanA/RT-1-tetramer to sort a polyclonal CTL line, Dunbar et al. (1998) found greater lysis of peptide-coated T2 cells and no background killing compared to non-sorted CTL. Using the PR1-tetramer, a homogeneous population of PR1-sorted CTL could be obtained for use in adoptive immunotherapy to treat patients with leukemia.

DNA polymorphism is found within exon 3 of Pr3. Since allogeneic CTL responses directed against leukemia also involve differences in minor antigens (mH) between donor and recipient, the Pr3 coding region was searched for potential polymorphisms. Twenty-eight HLA-identical donor-recipient pairs undergoing BMT were studied. DNA was prepared from frozen samples using the Wizard Genomic DNA Preparation Kit (Promega). Each of the 5 exons of Pr3 was amplified using specific primers (Table 8) and analyzed by PCR-SSCP (sequence specific conformational polymorphism). The PCR mixture was then analyzed with only 0.5 mCi of [³²P] dATP per reaction. The amplifications were carried out according to the “touchdown” protocol under the conditions summarized in Table 8.

PCR products were reamplified with the same primers to which were added an M13 sequencing primer tail (M13 -21 5′-TGTAAAACGACGGCCAGT-3′ (SEQ ID NO:14) for the forward primer and M13 Reverse 5′-CAGGAAACAGCTATGACC-3′ (SEQ ID NO: 15) for the reverse primer). The new PCR product was directly sequenced using the ABI Dye primer Cycle Sequencing Kit with the M13-21 and Reverse sequencing primers. Several independent sequences of each of the selected individuals were aligned and compared.

TABLE 8

Oligonucleotide Primers and PCR ™ conditions
used for the amplification of the PR3 exons

| | | | | | | | |
|---|---|---|---|---|---|---|---|
| | | | SEQ | | | | |
| | Amplified | | ID | Location | Size | No. of | Annealing |
| Name | exon | Sequence 5′ - 3′ | NO: | (base#)^{a} | (bp) | Cycles | temp (° C.) |

| | | | | | | | |
|---|---|---|---|---|---|---|---|
| P1-F | | ACT CAA CTC CGT CTG GCA TT | 16 | 348 | | 15 | 70-55 |
| P1-R | 1 | TGA TGA CCT CGT GGT GGA TA | 17 | 548 | 201 | 20 | 55 |
| P2-F | | GCT CCC TGA CGC CTG GAC TC | 18 | 2801 | | 5 | 70-65 |
| P2-R | 2 | ACG GGG CTT AGC TGG GTC CT | 19 | 3097 | 297 | 25 | 65 |
| P3-F | | CCG GGG AGG ACC CAG CTA AG | 20 | 3072 | | 10 | 65-55 |
| P3-R | 3 | GGT CGT GGC CCG GTA TAC AG | 21 | 3496 | 425 | 25 | 55 |
| P4-F | | TTT GAG GTG GTG GGT GTG GT | 22 | 5137 | | 5 | 70-65 |
| P4-R | 4 | AGG CAC AGC ATG AAG CCA CA | 23 | 5561 | 425 | 25 | 65 |
| P5-F | | TCA GGT GGC CCT GAT GGG TG | 24 | 6577 | | 5 | 70-65 |
| P5-R | 5 | TCG AGG GTT TGG AGC CAG GC | 25 | 6840 | 264 | 30 | 65 |

^{a}According to (Sturrock, et at)

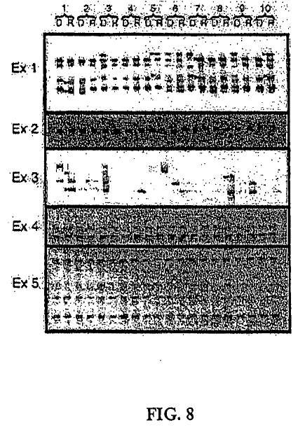

Genotype of donors and recipients was determined using Sequence Specific primer (SSP) PCR. The PCR™ products were run in 2% agarose gel stained with Ethidium Bromide. Different patterns between individuals were noted in exons 1 through 4, but only exons 1 through 3 showed differences between donors and recipients of the same pair, as shown in FIG. 8. Since the amplified DNA product also contained portions of the flanking intronic regions and since all of the base differences in the coding region may not result in amino acid differences, the amplified products from donor-recipient pairs showing donor specific bands were sequenced. These results show that the Pr3 gene is polymorphic for the following reasons: (1) The sequences as determined from the intronic regions conflicted several times with the published genomic sequence; (2) two different polymorphic sites were found in exons 1, 2, and 3 that explained the multiple patterns observed on autoradiography; (3) only one DNA polymorphism, in exon 3, was found to encode for an amino acid polymorphism in the deduced amino acid sequence (Table 9). This polymorphism encodes for either an isoleucine (ATT) or a valine (GTT) at position 119 of the amino acid sequence.

TABLE 9
Polymorphisms in the Pr3 gene as determined by direct gene sequencing

| | | | aa | |
|---|---|---|---|---|
| | Position | Base change | Change | Remarks |
| 1 | 390 | C or T | No | Outside coding Region (5′ UT) |
| 2 | 506 | C or T | No | Outside coding Region (Intron I) |
| 3 | 2827 | A or C | No | Outside coding Region (Intron I) |
| 4 | 3059 | A or G | No | Outside coding Region (Intron II) |
| 5 | 3393 | A or G | Val or | Exon 3 |
| | | | Ile 119 | |
| 6 | 3456 | A or C | No | Outside codign Region (Intron III) |
| 7 | 5179 | A or G | No | Outside coding Region (I(ntron III) |
| | | | | Found only in one donor |

Sequencings of the first 4 exons have been submited to Genbank (accession numbers: AF015446; AF015447; AF015448 and AF015449).
Aa: amino acid;
Ile; isoleucine;
Val: valine;
UT: untranslated.

Peptides that span the Pr3 polymorphic site bind to HLA-A2.1. Next, it was determined whether the polymorphism, as discussed above, could lead to the expression of different peptides that could bind to HLA molecules. Anchor motifs contained in the peptides spanning the polymorphic region of exon 3 were then looked for that could bind to several HLA class I molecules including HLA-A1, -A*0201, -A*0205, -A3, -A11, and -A24. There are two 10 amino acid peptides (KLNDILLIQL (SEQ ID NO:8)) or KLNDVLLIQL (SEQ ID NO:9)), named PR7I and PR7V, respectively) spanning the polymorphic site, at amino acid 115-124 which possess anchor motifs capable of binding to HLA-A2. Both peptides have the same predicted binding half-lives of 705 mn and 126 mn for HLA-A*0201 and -A*0205, respectively.

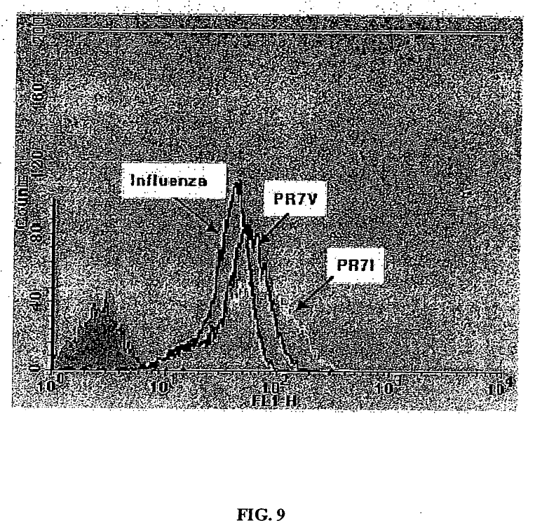

The ability of these peptides to bind to HLA-A*0201 was then tested in T2 cells by measuring surface HLA expression. Peptides PR7I and PR7V, together with control influenza peptide, were each pulsed onto T2 cells for 18 hr and the subsequent HLA-A2.1 expression was measured by flow cytometry. T2 cells pulsed with either PR7I or PR7V demonstrated strong peptide binding with fluorescence intensities greater than that of the positive control peptide (Influenza B Nuclear Protein; FIG. 9). Low background MHC class I expression was demonstrated by the low fluorescent intensity of the cells without added peptide.

It was previously shown that the PR1 self peptide from Pr3 is immunogenic, and these data suggested that T cell responses may be elicited against polymorphic differences in Pr3 as well. These differences may be used as a basis for designing leukemia-specific adoptive T cell therapy of myeloid leukemia.

### Example 5

### PR1 is Derived from Proteinase 3 and Neutrophil Elastase Proteins

The leukemia-associated antigen PR1 is derived from both proteinase 3 and neutrophil elastase proteins. The inventors have shown that killing of leukemia cells by PR1-specific CTL correlates with P3 overexpression. However, the PR1 sequence is also contained within neutrophil elastase (NE), which is also aberrantly expressed in leukemia cells. To determine whether PR1 is processed and presented from either protein, P3- and NE-transfected B cells were used as target cells in a proliferation assay with PR1-specific T cells. Full-length P3 and NE cDNA was cloned from the promyelocytic HL-60 cell line based on published sequences. The sequences were subcloned into an EGFP-containing vector (pCMS-EGFP) with a CMV promoter to drive constitutive expression. The B cell line, HMy-CIR, previously stably transfected with the HLA-A*0201 gene, was transfected with P3-pCMS-EGFP and NE-pCMS-EGFP by electroporation for 24 hr. GFP-expressing cells (P3-HMy.CIR-A2 and NE-HMy.CIR-A2, respectively) were selected by FACSorting and high transfection levels were confirmed by fluorescence microscopy. Quantitative real time-PCR showed mRNA transcripts from one of two selected P3-transfected clones were only 2.6-fold less than HL-60 cells (p=0.07), and only 1.5-fold less in a second clone (p=0.3). By contrast, NE transcript numbers from a transfected clone were 7-fold less than in HL-60 cells. Western blotting confirmed protein expression in the NE-HMy.CIR-A2-transfected cells, although expression was similarly less than in HL-60 cells. PR1-specific CTL lines elicited from two healthy donors showed stimulation indices (SI) of 1.3 and 2.4 when co-incubated with P3-HMy.CIR-A2 target cells during BrDU incorporation, confirming that PR1-CTL recognize PR1 peptide processed and presented from P3. Interestingly, PR1-CTL also recognized NE-transfected cells, with SI's of 1.5 and 2.1. No recognition of non-transfectants or parent HMy.CIR (A2-) was observed. These results demonstrated that, in addition to P3, PR1 is also processed and presented by NE, which may result in enhanced immunogenicity of the peptide compared to peptides derived from a single protein. Redundancy of proteins may also lessen the impact of tumor-loss variants after PR1-based immunotherapy.

### Example 6

### BMT Patients with Myeloid Leukemia Tested for CTL Immunity to PR1

The data in this study support that PR1 could be used as a target antigen to stimulate both active and passive leukemia-specific immunity. In this study, PR1 will be given as a vaccine with incomplete adjuvant to boost leukemia immunity, and in additional clinical trials PR1-specific CTL will be selected and expanded ex vivo with the PR1 antigen for the production of leukemia-reactive CTL. PR1-specific CTL will be used alone or combined with existing treatments, such as allogeneic BMT, to produce a GVL effect. PR1-specific donor-derived CTL added to a previously CD3 T cell-depleted graft, for instance, may allow selective GVL reactivity without GVHD. In this study, it has been shown that normal donors have existent CTL immunity to PR1, an HLA-A2-restricted 9 amino acid peptide (aa 169-177) derived from proteinase 3 (Pr3). Pr3 is a protein contained within the azurophil granules of myeloid lineage cells and is overexpressed in many patients with myeloid leukemia.

CTL responses to PR1 will be examined by determining CTL precursor (CTLP) frequency using microtiter limiting dilution analysis. As a first step to determining the clinical significance of CTL responses to PR1, blood and bone marrow samples will be examined from patients and their marrow donors prior to transplant, and at three, six, and nine months after transplant. Recently, it has been found that some patients with CML that have had a molecular relapse after allogeneic BMT have both Ph+ and Ph− populations of host-derived hematopoietic progenitors present, and that after DLI alone were given to patients the host-derived Ph− progenitors reconstituted normal hematopoiesis. This suggests that the allogeneic DLI that produced the remission might target CML independent of minor antigen differences between the donor and the recipient. If a CTL response to PR1 is contained within the GVL response, it is expected that CTLPf would be low in the donor, lower still in the recipient, but that it would increase following BMT. Alternatively, a high CTLPf against PR1 present in the recipient prior to BMT might indicate leukemia immune escape, which could be addressed separately as detailed below. Because of the limited availability of patient material, especially bone marrow, it is important to use the microtiter techniques described herein to determine CTLPf by limiting dilution analysis (LDA) using Terasaki trays.

PBMC and BM from HLA-A2.1+ patient samples will be used to test for existing T cell reactivity toward PR1 by using a limiting dilution CTLPf assay (Hensel et al., 1999). PBMC and bone marrow mononuclear cells (BMC) will be obtained by Ficoll Hypaque separation and either used fresh, or frozen in liquid nitrogen for future use. Samples from HLA-A2.1 positive patients with CML, AML, and MDS will be collected. PBMC and BM from the HLA-matched normal BMT donors to those patients will also be collected, cryopreserved, and assayed as well. The previously frozen samples will be thawed and washed three times in serum-free CM and counted. A total of 19.5 million cells will be prepared for the assay (7.5 million stimulator cells, and 12 million responder cells). One million additional PBMC will be used to generate phytohemagglutinin (PHA-P; Sigma Chemical, St. Louis, Mo.) stimulated blasts to be used as target cells on day 10 of the assay. One million unrelated HLA-disparate PBMC (third party) will also be used as a positive control, with half of the total number used as stimulator cells and the other half as responder cells.

PHA blasts will be generated by placing 10⁶ cells in 1.0 mL of CM supplemented with 10% fetal bovine serum (FBS; Sigma Chemical) in a 25 cm² flask. Five μL/mL of PHA-P will be added, and the cells will be cultured in 5% CO₂ at 37° C. On day 4, 6, and 8 of the culture, additional CM+10% FBS with 5 μL/mL PHA-P and 500 IU/mL rhIL-2 will be added. Cells will be counted and cultures will be maintained at 10⁶ cells/mL. These cells will be used as target cells on day 10 of the CTLPf assay.

PBMC responder cells (from patient samples) will be plated into high profile Terasaki Trays (Robbins Scientific, Sunnyvale, Calif.) at 6 dilutions (from 5×10⁴ to 5×10⁵ PBMC/well) using 24 replicates at each dilution in CM+10% human AB serum (AB; Sigma Chemical). Third party responders (from normal donors) will be plated at a single dilution (5×10⁴ PBMC/well). Patient PBMC stimulator cells will either be pulsed with peptide (PR1 or FLU, the influenza nuclear protein described previously, as a positive control peptide) at 20 μg/mL or no peptide for 90 min at 37° C., washed once with serum-free CM, then irradiated with 2500 cGy. These cells will then be plated into the wells containing the responder cells, as well as 24 additional wells of stimulators alone. On days 3 and 7, 60 IU/mL of IL-2 will be added to each well. On day 10 the previously prepared PHA blasts from the same patient (as target cells) will either be pulsed with PR1 at 20 mg/mL or no peptide for 90 min at 37° C., washed once with serum-free CM, and then labeled with Calcein AM (Molecular Probes, Eugene, Oreg.) as described in the cytotoxicity experiments above. One thousand target cells per well will be plated, lightly centrifuged at 800 rpm for 1 min, and then cultured in 5% CO₂ at 37° C. for 4 hr. Five microliter of Fluoroquench EB Stain-Quench Reagent (One Lambda, Canoga Park, Calif.) will be added to each well, and the plates will be analyzed for fluorescence emission using an automated Lambda Scan (One Lambda, Canoga Park, Calif.).

The mean plus 3 standard deviations of the 24 wells containing the stimulators alone will be determined as the cut-off value for background fluorescence. Any experimental well less than the cut-off value will be considered positive for lysis, and from the fraction of negative wells at the various responder cell dilutions, the frequency of CTLP will be calculated using the maximum likelihood method based on Poisson probabilities. The calculated CTLP frequencies will then be analyzed with respect to the amount of Pr3 expression in the patient's blast populations, since the amount of specific lysis of PR1-specific CTL in normal donors correlated with the amount of Pr3 overexpression in the target leukemia cells in the previous studies (Molldrem et al., 1996).

Determining the percent of PBMC and BM specific for the PR1 peptide. CTL responses to PR1 will be examined determining the percent of peripheral blood mononuclear cells (PBMC) and bone marrow cells (BM) that are specific for the HLA-A2.1-bound PR1 peptide by FACS analysis using a specific PR1-HLA-A2 tetramer (PR1-tetramer) linked to phycoerythrin (PE). The PR1-tetramer will be used to identify the fraction of CTL that recognize HLA-A2-bound PR1 in patient and donor PBMC and BM before transplant and again 6 months after transplant. This will be easier to study using patients that receive peripheral blood grafts since more patient material is available for the larger number of lymphocytes needed for flow cytometry determination of the percent of PR1-specific CTL.

PR1-specific CTL, determined using the combination of PR1-tetramer plus CD8, will be analyzed by using three and four color FACS analysis to simultaneously determine the phenotype of the PR1 -specific CTL. The cells will be analyzed for state of activation using CD28, CD69, and CD25. Memory CTL will be evaluated using CD45RO antibody. It is important to determine whether any PR1-specific CTL that are found in the leukemia patients are memory cells in an inactivated state which would suggest either that the leukemia is not a potent immunogen or perhaps the CTL are incapable of responding. By also determining Pr3 expression and surface phenotype of the leukemia cells (including MHC I and II, -CD40, CD54, CD80 and CD86), immunogenic potential can be better assessed.

Since the precursor frequency of PR1 -specific CTL is quite low even in the normal donors previously examined, PR1-specific CTL may not be detectable by FACS analysis because of the low total number of cells available in the patient samples. In addition, patient lymphocytes may be incapable of responding to PR1 peptide due to tolerance or anergy. By expanding PR1-specific CTL lines in vitro using T2 cells pulsed with PR1 peptide this possibility will be investigated. Starting with cryopreserved patient PBMC and BM, 10 million mononuclear cells will be thawed and washed 3 times with PBS. These cells will be placed into culture with 5 to 10 million T2 cells (previously pulsed with 10 μg/mL of PR1 for 90 min at 37° C.) in CM+10% HS at 37° C. and 5% CO2 for one week and then re-stimulated with PR1-pulsed T2 cells on day 7. On day 8, IL-2 at 20 IU/mL will be added to the cultures and this process will be repeated at two and three weeks of cell culture. The cells at the end of three weeks in culture will be collected and used in micro-cytotoxicity assays using Calcein AM-labeled T2 cells as targets. T2 target cells will be pulsed with 10 μg/mL of PR1, control Flu peptide, or no peptide for the cytotoxicity assays. A CTL line established from an HLA-matched normal donor using the same methods will be used as a positive control in the cytotoxicity assay, since this is an established method.

CTL responses to PR1 will be examined determining the frequency of cells secreting cytokines in response to PR1 peptide recognition using the ELISPOT assay. Cytokine-secreting cells will be measured in response to PR1-coated targets by using the ELISPOT assay. Recent clinical trials have used methods such as precursor frequency analysis by limiting dilution based on cytotoxicity (⁵¹Cr), lymphocyte proliferation (³H thymidine uptake), as well as cytokine responses by measuring single cell interferon-γ or TNF-α secretion (using the commercially available ELISpot assay, Mabtech, Nacka, Sweden). Each of these methods measure different lymphocyte functional responses, and clinical response to tumor vaccines does not always correlate with these in vitro measurements (Nelson et al., 166; Rosenberg et al., 1998).

Lalvani et al. (1997) have determined, using the enzyme-linked immunospot (ELISPOT) assay, that memory CD8⁺ T lymphocytes are capable of rapid effector function (i.e. 24 hr) when triggered with exposure to cognate peptide in the absence of cytokines. The frequency of CTL as measured by ELISPOT has been shown to correlate closely with the number of CTL that stain with peptide-specific tetramers in FACS assays (Dunbar et al., 1998), although limiting dilution assays (LDA) often yield lower frequencies by comparison. Others have found that 14-day expansions of PBMC against peptide-coated target cells results in a better correlation of LDA with the ELISPOT assay (Scheibenbogen et al., 1997). These results suggest that LDA, which is highly dependent on culture conditions and relies on the presence of proliferating cells, may underestimate the actual number of peptide-specific CTL present.

ELISPOT assays will be performed in 96-well polyvinylidene difluoride backed plates (MAIP S 45; Millipore, Bedford, Mass.). These wells will be coated with 15 μg/mL of anti-IFN-γ mAb 1-DIK (Mabtech, Stockholm, Sweden) overnight at 4° C. Plates will be washed 6 times with CM and blocked with CM+10% HS for 1 hr. PBMCs will be thawed and washed 3 times with PBS and suspended in CM+10% HS. PBMCs will be added in 100 μL CM+10% HS per well to the precoated plates at 5×10⁵/well, in duplicate wells. For assays performed in parallel with CTLPf by LDA, duplicate wells with 5×10⁵ and 2.5×10⁵ PBMCs/well will be used.

Detection of peptide-specific T cells from freshly isolated PBMCs will be performed using autologous PBMCs themselves to present PR1 peptide. This will avoid responses been elicited from T cells of other specificities if heterologous B cell lines (such as EBV-transformed lymphocytes) were used as target cells for peptide presentation. Peptides will be added at 10 μg/mL. T2 cells will be used in some assays as target cells and compared to the autologous PBMCs. In this case, T2 cells will be pulsed with 10 μg/mL of PR1 peptide for 90 min and washed 3 times prior to plating with responder PBMCs.

The plates will then be incubated for 6 hr at 37° C., 5% CO₂ and then arrested by shaking off the contents and washing 6 times with PBS 0.05% Tween 20 (Sigma Chemical, St. Louis, Mo.). Next, 100 μL of 1 μg/mL of the biotinylated anti-IFN-γ mAb 7-B6-1 biotin (Mabtech, Stockholm, Sweden) will be added. After 3 hr of incubation, plates will be washed six times more and a 1:1000 dilution of streptavidin alkaline phosphatase conjugate (Mabtech) will be added to the wells and the plates then incubated at room temperature for a further 2 hr. Next, the wells will again be washed 6 times and 100 [L of chromogenic alkaline phosphatase substrate (Bio Rad Labs, Hercules, Calif.), diluted 1:25 with deionized water, will be added. After 30 min, the colorimetric reaction will be terminated by washing with tap water and the plates will be air dried.

Enumeration of the IFN-γ-producing spot-forming cells (SFCs) will be performed using a stereomicroscope under magnification of 20. Only large spots with fuzzy borders will be scored as SFCs as per convention (Klinman, 1994). Responses will be considered significant if a minimum of five SFCs are present per well, and additionally, this number is at least twice that in negative control wells (without added peptide). Counting SFCs allows the added advantage of estimating the frequency of responding CTL.

If no SFCs are seen, then the time of exposure to peptide will be increased in a time course experiment up to 48 hr. If no SFCs are yet produced, this could be an indication that a secondary response could not be produced and a primary response will be investigated. In this case, PBMCs will be pulsed with PR1 peptide and exposed as stimulators to autologous PBMCs at a 1:1 responder: stimulator ratio on two occasions over 7 to 10 days with low-dose IL-2 added to each well at 60 IU/mL before the ELISPOT assay is performed.

In addition, since others have demonstrated that TNF-α is sometimes produced by greater numbers of peptide-specific CTL than γ-IFN, the ELISPOT technique will also used to quantify the number of SFC in a TNF-a assay under similar conditions (Herr et al., 1996). By making use of the Perseptive Biosystems Cytofluor 4000 plate reader requested in the budget of this proposal, the CTLP frequency by LDA, cytotoxicity experiments, and ELISPOT assays can all be performed using this same instrument (Herr et al., 1997).

Evaluation of Pr3 expression and surface phenotype of myeloid leukemia target cells. Cytoplasmic Pr3 expression will be determined in BMC from each of these patient samples, based on cytoplasmic staining and subsequent analysis using flow cytometry using the method previously described (Molldrem et al., 1996). Cells will also be surface-labeled with anti-CD33, CD34, and CD14 monoclonal antibodies (Becton-Dickinson, San Jose, Calif.) in order to correlate the developmental stage of the blast population with Pr3 expression. In addition, cells will be labeled for MHC class I and II, CD80 (B7.1) (ImmunoTech S.A., Marceilles Cedex, France), CD86 (B7.2) (ImmunoTech), and CD54 (ICAM-1) (Becton Dickinson) to evaluate the potential of the blasts as suitable CTL targets. Three color staining will allow for cell subset analysis. One million cells will be studied for each combination of antibodies. The cells will be washed in serum-free CM, permeabilized using Ortho PermeaFix for 30 min at room temperature, washed twice in serum-free CM, and then indirectly stained with an antibody to Pr3 (Accurate Chemicals, Westbury, N.Y.) and a secondary FITC-conjugated antibody. This will be followed by an additional wash with serum-free CM and surface labeling of the other markers using direct staining with PE- or APC-conjugated antibodies at 4° C.

As previously discussed, a high CTLPf in the recipient pre-BMT may indicate leukemia immune escape. This may be due to decreased expression of MHC 1 (Dermime et al., 1997, decreased co-stimulatory molecules (Matulonis et al., 1995; Boussiotis et al., 1995), decreased Pr3 expression in the leukemia (Molldrem et al., 1996), or possibly a TCR signaling defect in the CTL (Boussiotis et al., 1996). In addition, a failure to increase CTLPf after BMT may also be due to any of these mechanisms. By examining expression of these molecules and correlating CTLPf with the number of PR1-specific CTL present by PR1-tetramer labeling, insight into possible failure of a CTL immune response to PR1 will gained. For instance, a low CTLPf but a high percent of PR1-specific CTL by flow cytometry may indicate a defect in CTL target recognition, which may be further investigated by examining TCR—chain tyrosine phosphorylation by Western blot analysis.

Humoral immune responses to Pr3 will be examined by measuring antineurophil cytoplasmic antibody (ANCA) titers. In Wegener's granulomatosis, the ANCA IgG titer correlates closely with disease activity. Since T lymphocytes taken from biopsy sites of active vasculitis in these patients show proliferation in response to Pr3, and since T cell help is required for IgG production, it is possible that measuring ANCA titers may be an indirect measure of Pr3-directed activity of T lymphocytes.

ANCA titer has not been formally examined in patients with myeloid leukemia. An inexpensive and reliable assay is commercially available and used in clinical laboratories to evaluate ANCA titers. ANCA titers will be determined in all myeloid leukemia patients enrolled on phase I/II clinical vaccine trials as well as the transplant patients and their donors will be investigated using the above assays to measure cellular immunity. Patients of all HLA types will be evaluated for ANCA positivity.

### Example 7

### Determining whether PR1 can Elicit Specific Antileukemia Immunity in Patients with Myeloid Leukemia

It was next determined whether PR1 can be used to elicit specific antileukemia immunity in patients with myeloid leukemia. Leukemia offers several advantages as a model disease for vaccine development and evaluation. First, since the leukemia cells circulate in the peripheral blood the tumor cells are readily available for study without the need for repeated invasive biopsies which allows for close monitoring of any changes in the tumor phenotype. In addition, the lymphocytes under study also reside in the peripheral blood in constant contact with the malignant cells. These are obvious advantages to the study of immune responses in the treatment of solid tumors.

Enhancing CTL reactivity by PR1. A phase I/II clinical protocol using the PR1 peptide in combination with incomplete Freund's adjuvant (IFA) will be employed to vaccinate patients with myeloid leukemia to determine whether CTL reactivity to PR1 peptide can be enhanced by subcutaneous vaccination with the PR1 peptide combined with incomplete Freund's adjuvant (IFA) every 3 weeks for 3 injections. CTLP frequency will be measured in PBMC and BM before and after vaccination using microtiter limiting dilution analysis. IFA has been used in one successful clinical trial using gp100 to vaccinate patients with melanoma (Rosenberg et al., 1998).

Up to 60 HLA-A2+ patients (as confirmed with BB7.2 antibody labeling) with CML, AML, and MDS will receive a deep subcutaneous injection every 3 weeks for 9 weeks. Patients will be randomized to three dose levels of the PR1 peptide (0.25 mg, 0.5 mg, and 1.0 mg) with a fixed amount of IFA, and then followed in cohorts of 4 for toxicity and evidence of an immune response to PR1 as measured in vitro using the CTLPf assay by LDA. Patients will be randomized to three different doses of vaccine, since it is not clear that increased dose will result in a greater anti-tumor response. In fact, if toxicity of a vaccine is related to the augmentation of the immune response, that augmentation may be elicited by repeated immunization rather than increasing the dose of the immunizing antigen. The “dose escalation” of a vaccine may not lie in the quantity of peptide delivered, but rather in the number of exposures to antigen, i.e. the number of immunizations. In fact, in a study of immunization with TCR derived peptides for the treatment of multiple sclerosis there was an indication that the peptide-specific T cell immune response was suppressed when the amount of peptide injected was greater than 1000 μg (Bourdette et al., 1994).

In addition, immune responses will be investigated using the PR1-tetramer. A one log increase (compared to study entry) in CTLPf, or any increase in the percent of PR1-specific CTL by FACS analysis measured at any time over the 9 weeks will be considered evidence of an immune response to PR1. These assays will be performed using samples of bone marrow and peripheral blood from patients enrolled on this protocol. Standard criteria for complete and partial remissions will be used to judge clinical responses.

Using the methods described, the CTLPf (by LDA) and the percent PR1-specific CTL measured using the PR1-tetramer will be used to assay immune responses in the patients. Approximately 20 million PBMC will be used in both assays derived from 100 ml peripheral blood samples before and 3 weeks after the last vaccination. Further experiments will be performed to evaluate any non-responsiveness found using both these assays.

An important aspect in the design of any phase I/II cancer vaccine trial targeting a self tumor antigen is the selection of the patients to be immunized. Toxicity such as vasculitis is possible and therefore patients with advanced stage leukemia will be eligible for this initial study. However, determination of safety requires that an immune response be mounted, and far advanced patients often lack a competent immune response. Therefore, patients with all types of myeloid leukemia at various stages of disease including patients with chronic phase CML who will have a stable low-leukemia burden and normal lymphocyte counts will be examined. It is anticipated that the chronic phase CML patients will be those without the option of of allogeneic BMT, who may be failing other forms of therapy such as interferon with or without chemotherapy, and who are therefore terminally ill. Patients with CML offer the further advantage over solid tumor patients that molecular studies of the amount of Ph chromosome can be used to judge responses in the chronic phase patients with low tumor burden. PCR analysis, which is performed routinely on all CML patients to follow the disease course, will be performed at 3-week intervals during the course of the study, and again one month after the last vaccine.

In order to increase the likelihood of producing an immune response against the leukemia, a subcutaneous injection of 75 μg of GM-CSF will be administered into each vaccine injection site to enhance the adjuvant effect. GM-CSF as an adjuvant has produced greater immune responses in animal models of other hematological malignancies (Kwak et al., 1996).

Phase I trials using bcr-abl junction region peptides to vaccinate patients with CML are being conducted by other investigators. These investigators are using a combination of 5 HLA-A3 and HLA-All-restricted peptides from the b3a2 translocation region with the adjuvant QS-21 in a dose-escalation trial design starting with 10 μg of peptide. Thus far, 5 patients treated with 10 μg and 5 patients treated with 30 μg (the next dose level) have not had any clinical response to the vaccine. By using the conventional ⁵¹Cr-release cytotoxicity assay, these investigators have not found in vitro evidence of an immune responses to the peptides thus far.

Thus, as provided in the present invention, the PR1 peptide vaccine has greater potential to elicit immunity compared to bcr-abl junction region peptides for several reasons. First, the pre-clinical data regarding bcr-abl junction region peptides indicates that CTL elicited in vitro against these peptides do not demonstrate cytotoxicity against fresh leukemia target cells, but only to leukemia cells that have been pre-pulsed with the target peptides. In contrast, PR1-specific CTL show cytotoxicity and leukemia progenitor inhibition of fresh leukemia cells with no prior peptide labeling of the target cells (FIG. 2). Second, clinical trials showing responses to melanoma peptide vaccines have used 10-fold higher peptide doses to vaccinate patients than what is used in the b3a2 peptide vaccine trial. The PR1 vaccine trial will use peptide doses similar to those used in the melanoma vaccine trials. Third, the ⁵¹Cr-release CTLPf by LDA that was used to evaluate an immune response to the b3a2 peptides is less sensitive than the mirco-titer CalceinAM-based assay of the present invention. Lastly, the PR1 vaccine trial will include patients with all types of myeloid leukemia, thereby broadening the opportunity to find immune responses.

Potential non-responsiveness to PR1 will be evaluated by generation of PR1-specific T cells in myeloid leukemia patients. In this phase I/II study, whether PR1-specific CTL responses can be elicited in patients by using the peptide vaccine will be determined. In those patients where no immune response is elicited, or in those patients that develop an immune response but do not have a clinical response to the vaccine, it will be important to know whether PR1-specific CTL from those patients can recognize and/or kill autologous leukemia cells. These patients will be investigated further to determine whether a PR1-specific CTL can be elicited from PBMC in vitro using methods described herein. In these patients it is expected that a PR1-specific CTL will not be elicited in vitro, or any CTL that could be expanded might not be able to recognize or kill the leukemia.

PBMC from amongst patients with CML, AML, and MDS that do not develop an immune response to the vaccine will be selected in order to identify any differences based on disease. Fifty million T2 cells will be washed in serum-free CM three times and suspended at 10×10⁶ cells/mL in 50 mL conical tubes. PR1 peptide, synthesized to >95% purity (Biosynthesis, Lewisville, Tex.), will be added to the T2 cells at 10 μg/mL and the T2 cells will be placed in a humidified incubator at 37° C. for 90 min. The PR1-pulsed T2 cells will be irradiated with 7500 cGy, washed twice with serum-free CM, and combined at a 1:1 ratio with patient PBMC in 50 mL of CM+10% human AB serum (10% AB) and placed in a 75 cm² tissue culture flask. Media will be replaced as necessary, and at day 7 the PBMC will be washed with CM+10% AB and recombined at a 1:1 ratio with fresh T2 cells (similarly pulsed with PR1, irradiated, and washed). On day 8, 20 IU/mL of recombinant human interleukin-2 (rhIL-2) (Biosource International, Camarillo, Calif.) will be added to the culture. After 14 days in culture a third stimulation will be performed with PR1 -pulsed T2 cells, followed on day 15 by addition of rhIL-2. A fourth stimulation will be performed on day 21 followed on day 22 by the addition of 20 IU/mL rhIL-2. After a total of 25 to 27 days in culture, the PR1-stimulated T cells will be tested for peptide-specific cytotoxicity toward T2 cells and leukemia cells from autologous and allogeneic BMC.

A semi-automated mircotiter cytotoxicity assay, identical to that used in the previously published studies, will be used to determine specific lysis. Effector cells grown in the presence of PR1-pulsed T2 cells will be prepared in doubling from 6×10³ to 25×10³ cells/well and will be plated in 40 μL, 60-well Terasaki trays (Robbins Scientific, Sunnyvale, Calif.) with six replicates per dilution. Target cells (T2 cells±PR1, autologous marrow-derived leukemia cells, or marrow derived from normal donors) will be thawed and washed three times in serum-free CM then suspended at a concentration of 2×10⁶ cells/mL and stained with 10 mg/mL of Calcein-AM (CAM; Molecular Probes Inc, Eugene, Oreg.) for 60 min at 37° C. After washing three times in CM+10% AB, target cells will be resuspended at 105 cells/mL. Wells with target cells alone and medium alone will be used for maximum (max) and minimum (min) fluorescence emission, respectively. After 4 hr incubation at 37° C. in 5% CO₂, 5 μL FluoroQuench EB Stain-Quench Reagent (One Lambda, Inc, Canoga Park, Calif.) will be added to each well and the trays will be centrifuged for 1 min at 60 g before measurement of fluorescence using an automated Lambda Fluoroscan (One Lambda, Inc). A decrease in the fluorescence emission is proportional to the degree of lysis of target cells, once the hemoglobin contained in the FluoroQuench reagent quenches the released dye. The mean and standard deviation fluorescence from the 6 wells at each E:T ratio will be calculated, and the percent lysis will be calculated.

PR1 specificity will be confirmed by using Flu peptide-coated T2 cells as a negative control target cell population in the cytotoxicity assay. Autologous BMC and HLA-A2+ BMC from a normal donor will be prepared similarly and also used as target cells to test for leukemia-reactivity. In addition, patient PBMC will be used in parallel to generate Flu-specific CTL lines which will be similarly tested for lysis of Flu peptide-coated T2 cells. In the case where CTL immunity is preserved against the Flu peptide, but can not be elicited against PR1, then comparison to any existent pre-vaccine PR1 immunity (as measured by CTLPf, PR1-tetramer positive CTL, or PR1-specific CLT by ELISPOT) would assess the development of tolerance toward PR1.

To confirm HLA-A2.1 specificity in these experiments, 100 μL/mL of the HLA-A2-specific blocking antibody BB7.2 (ATCC; Rockville, Md.) will be co-incubated with 1×10⁶ PR1-coated T2 cells for 30 min at 37° C. prior to coincubation with effector cells. Starting PBMC and resultant T cell populations will be phenotyped for T cell subsets using the PR1-tetramer, anti-CD3, CD4+CD8, CD3+CD16+56, and anti-TCR-αβ (Becton Dickinson).

In order to elicit autologous PR1-specific cytotoxicity in vitro, an alternative approach that may be used will employ PR1-pulsed dendritic cells (DC) derived from adherent PBMC populations and grown for 7 to 10 days in IL-4 and GM-CSF in place of T2 cells to stimulate responder CTLs using PBMC from these patients (Nestle et al., 1998). In patients where a loss of immune response to PR1 is found, 100 μL of blood will be collected and PBMC isolated over a Ficoll-Hypaque density gradient as previously described. PBMC will be allowed to adhere to a plastic 75-cm² flask for 2 hr at 37° C. Non-adherent cells will be removed, and the adherent cells will be cultured for 7 days with GM-CSF (800 U/mL; Sandoz, Germany) and IL-4 (500 U/mL; PharMingen, Hamburg, Germany). Phenotypic changes will be monitored by light microscopy and flow cytometry will be performed to confirm the DC phenotype. Cells will be evaluated for high levels of HLA class I, HLA class II, and costimulatory molecules (CD80 and CD86) as previously described. PR1 will be used to pulse dendritic cells (DC) derived from the peripheral blood of normal donors, and these PR1-pulsed DC will be used like T2 cells in previous experiments to elicit PR1-specific CTL. PR1-specific lysis will be evaluated using these DC-generated CTL using assays discussed previously and compared with T2-generated CTL.

In additional experiments. Pr3 expression in patient BMC will be evaluated after the addition of interferon-γ, interferon-α, and GM-CSF. These cytokines are known to increase T cell MHC molecules and co-stimulatory molecules (Tsukada et al., 1997) and also increase Pr3 expression (Mayet et al., 1997; Sibelius et al., 1998). Thus, cytokine co-administration might lead to more effective anti-PR1 immunity (Dermime et al., 1997).

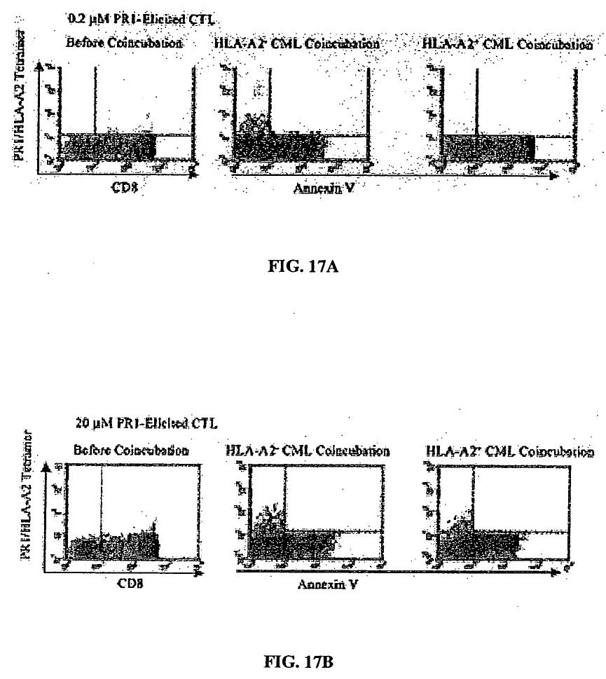

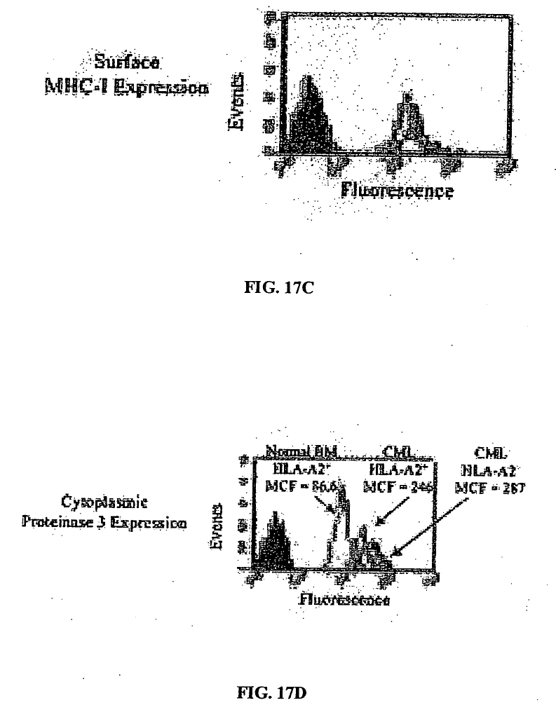

To determine whether a FACS-sorted population of PR1-specific CTL from HLA-A2 donors can produce enhanced lysis of autologous and allogeneic leukemia cells. As indicated in FIG. 17, the PR1-tetramer may be used to FACS sort for a homogeneous population of PR1-selected CTL that show high specific lysis of leukemia with no background lysis of normal marrow cells. Therefore, the PR1-tetramer will be used in the present invention to FACS sort homogeneous population of CTL from bulk culture CTL lines established at 2 to 3 wk of culture. These CTL lines will be generated using PBMC from HLA-A2.1+ patients with CML and AML as well as normal donors using PR1-pulsed T2 cells as antigen presenting cells. Using the PR1-tetramer, these cells will be FACS sorted and their state of activation and phenotype will be compared (by measuring CD28, CD69, CD25, and CD45RO) and their ability to lyse PR1-coated T2 and fresh autologous and allogeneic leukemia cells.

The results of these cytotoxicity experiments will be compared to the results obtained with bulk culture CTL as well as the population of CTL left behind after the sort procedure. PR1-sorted CTL that demonstrate cytotoxicity toward the leukemia cells, will also be evaluated for their ability to inhibit leukemia CFU-GM and normal HLA-A2.1+ CFU-GM using normal donor bone marrow cells and the methods previously described.

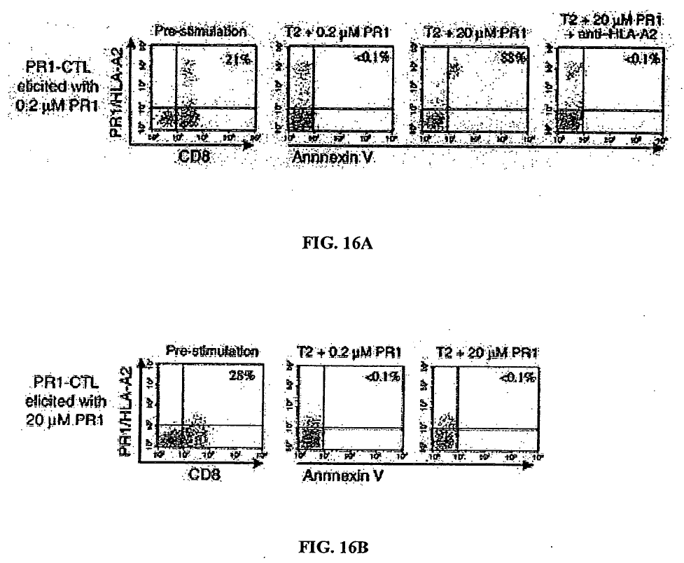

The PR1-sorted CTL, as shown in FIG. 16, contain some CD8+ cells that do not have apparent specificity for PR1. However, a much more homogeneous population of PR1-specific CTL are obtained using the PR1-tetramer, which will allow for more efficient cloning of these cells. Cells will be cloned by limiting dilution according the Poisson distribution where 0.3 cells/well will be plated into 96 well plates containing 1×10⁵ allogeneic PBMC (previously radiated with 7,500 cGy) in 300 μL CM+10% HS and 60 IU/mL IL-2. These wells will be placed at 37° C. and 5% CO₂ for 7 to 10 days. Media will be replaced after 7 to 10 days and wells that contain growing cells will be expanded and subsequently studied for specific lysis of PR1-coated T2 cells as previously described. These clones will also be evaluated for phenotype using the above antibodies.

Clones obtained using these methods will be used to investigate the Hmy2.CIR-A2 transfectants and other cells transfected with the Pr3 gene as discussed elsewhere in this application.

Similar to that demonstrated in previous studies (Molldrem et al., 1999), CTL from a patient with CML that demonstrate high specific lysis of PR1-pulsed T2 target cells was not elicited. Therefore, to overcome the apparent non-reactivity to this self-peptide in patients with leukemia FACS-sorted populations of PR1-specific CTL from normal donors will be investigated.

Normal donor-derived allogeneic PR1-specific CTL that are FACS sorted using the PR1-tetramer may be used for the safe adoptive transfer into leukemia patients selected only for the HLA-A2.1 allele as demonstrated using CTL specific for adenovirus-associated tumors in mice (Toes et al., 1996). This type of allogeneic therapy might allow for selective killing of host leukemia without the requirement of a traditional bone marrow transplant and without the requirement of a fully HLA-matched donor.

### Example 8

### Identifying Potential HLA-A2.1-Restricted Peptide Epitopes in PR3 to Elicit Leukemia-Reactive Human CTL using Immunological Methods

To determine whether CTL reactivity can be elicited against the remaining self peptides contained within Pr3 that are predicted to bind to HLA-A2.1. The development of effective peptide vaccines for leukemia will likely involve the identification of several peptide epitopes, since a single peptide might not be immunogenic in all individuals. It is likely that other peptides within Pr3 will also elicit CTL immunity, and using the same methods to find the PR1 peptide, seven additional peptides within Pr3 have been identified that contain the HLA-A2.1 binding motif (Table 4, PR3-PR9). These peptides have been identified to 95% purity (Biosynthesis Co.) and tested for HLA-A2.1 binding by performing flow cytometry for HLA-A2.1 expression on peptide-coated T2 cells using methods previously described. These peptides will be used to elicit CTL where peptide-coated T2 cells are radiated, pulsed with peptide, and used to stimulate PBMC from normal donors. These resultant CTL lines will be tested first for peptide-specific recognition of peptide-coated T2 cells (compared to non-coated T2), and then for lysis of fresh HLA-A2.1+BMC from leukemia patients (compared to BMC from normal HLA-A2.1+ donors). Blocking studies with antibody to HLA-A2.1 (BB7.2) and irrelevant Flu peptide-coated T2 cell targets will be used to confirm the allele-specificity in cytotoxicity assays. The relative binding affinities to HLA-A2.1 of these peptides compared to PR1 will be determined by incubating serial dilutions of PR1 plus each peptide with T2 cells and analyzing for surface HLA-A2.1 expression by flow cytometry. In this way, an IC₅₀ value will be determined for each peptide.

Since the peptides predicted to bind to HLA-A2.1 in Table 4 might be subdominant epitopes, CTL immunity toward peptide-pulsed T2 cells might be easily elicited but subsequent immunity toward HLA-A2.1+ leukemia targets might be lacking. In this circumstance, autologous dendritic cells will be substituted as antigen presenting cells for the T2 cell line and the resulting CTL will be examined for cytotoxicity toward HLA-A2.1+ leukemia targets.

For the peptides PR5-6 and PR8-9 that have relatively lower predicted binding affinities to HLA-A2.1, if only weak immunity toward peptide-pulsed T2 cells is elicited, substitutions will be made in the HLA-A2.1 anchor positions. At positions 2 and 9 of the peptide, leucine and methionine or valine and leucine will be substituted for the existing amino acids and these peptides will be used to elicit CTL immunity again using T2 cells. These amino acids are known to be relevant for high affinity binding to the HLA-A2.1 allele (Rammensee et al., 1995), and substitution of anchor amino acids has been shown to increase CTL lysis of the peptide-coated target cell (Parkhurst et al., 1996), presumably because of more stable binding of the peptide to the MHC class I heavy chain (Sette et al., 1994). Peptides that do not elicit any immunity will be considered not to be within the TCR repertoire of the donor PBMC.

To determine whether CTL reactivity to the PR1 peptide can be enhanced by single amino acid substitutions in the HLA-A2.1 anchor motif positions. As previously stated, it is known that certain amino acid substitutions in a peptide may enhance binding of the peptide to HLA-A2.1, which may subsequently enhance target recognition by CTL (Rosenberg et al., 1998). Thus, homologous PR1 peptides that contain single or double amino acid substitutions at the HLA-A2.1 anchor residues will be used to coat T2 cells and test for specific lysis. These peptides (Table 10) are predicted to have higher binding affinities to HLA-A2.1 based on the same algorithm used to predict the PR1 and PR2 peptides (Parker et al., 1994). These peptides will be synthesized (Biosynthesis Co.), tested for HLA-A2.1 binding using T2 cells, and tested in the mini-cytotoxicity assay where specific lysis will be compared to native PR1-coated T2 cells using TCL that are PR1-specific. The PR1-specific bulk culture CTL will be generated using the methods described herein.

If any additional HLA-A2.1-binding peptide from Table 10 is found that can be used to generate CTL responses, then combinations of PR1 with this peptide will be used to coat T2 target cells to test for specific lysis. Using CTL specific for this peptide, T2 cells will be coated with a fixed concentration of the peptide at 10 μg/mL, plus serial dilutions of PR1 (0.1 to 50 μg/mL) to test for potential interference with TCR recognition, as measured by reduced specific lysis at fixed E:T ratios. The results of these experiments plus the IC₅₀ of each of the peptides will be used to make comparisons of which are the possible dominant and subdominant peptides. This will be used to develop vaccines using combinations of peptides to stimulate CTL immunity (Nestle et al., 1998).

If peptides used to coat T2 cells from Table 10 do not result in greater cytotoxicity over PR1-coated T2 cell targets, then PR1-specific CTL that are obtained after PR1-tetramer sorting will be studied for their ability to recognize and lyse the PR1-variant peptide-coated T2 cells. Because these cells are a much more homogeneous population of CTL, they are expected be a more sensitive indicator of improved CTL immunity.

TABLE 10

Synthetic PR1-Like Peptides
with Amino Acid Substitutions

| | | | |
|---|---|---|---|
| Peptide | Amino Acid | SEQ ID | Predicted Binding |
| Name | Sequence | NO: | Half-Life (minutes) |

| | | | |
|---|---|---|---|
| PR1 | VLQELNVTV | 1 | 304 |
| PR1V6 | VLQELWTV | 26 | 1115 |
| PR1K8 | VLQELNVKV | 27 | 485 |
| PR1V6K8 | VLQELWKV | 28 | 1115 |
| PR1M2V6 | VMQELWTV | 29 | 805 |

To determine whether the HLA-A2.1-restricted peptides spanning the known single amino acid polymorphism in Pr3 can be used to elicit CTL immunity from the PBMC of donors that possess the opposite polymorphism. Based on previous studies, a polymorphism in the third exon of Pr3 was found that encodes for a single amino acid difference in peptides that can bind with high affinity to HLA-A2.1. The amino acid difference does not involve HLA-A2.1 the anchor regions, but it likely involves the region of the peptide recognized by the TCR. This polymorphism may therefore represent a new minor antigen that is restricted to hematopoietic tissue, and provide an ideal target for allogeneic adoptive immunotherapy strategies.

Therefore, these two peptides (PR7I and PR7V) will be synthesized and used to coat T2 cells for in vitro immunization of PBMC derived from the donors already known to have the opposite polymorphism using the methods to generate PR1 reactivity. The resulting CTL lines will be tested for specific lysis of T2 cells pulsed with either the original or the polymorphic peptide. These peptides will also be evaluated for lysis of leukemia cells from patients that have previously been determined to contain the opposite polymorphism. HLA-A2.1 blocking studies will confirm A2.1-restriction, and irrelevant Flu peptide will confirm peptide specificity. Lysis of these leukemia target cells without lysis of autologous marrow progenitors, will demonstrate PR7I and PR7V as the first potential new minor antigens found in humans.

In order to adequately estimate the ability of PR7I and PR7V to elicit CTL immunity, several PBMC donors will need to be studied since not all individuals would be expected to have T cells capable of recognizing the peptide. Furthermore, controls where CTL are elicited against the autologous PR7 peptide from the donors that contain that polymorphic peptide will need to be compared to CTL elicited from the donors that do not carry the polymorphism. If no reactivity can be found using any of these combinations using PBMC from three to four donors, then it is unlikely that either of these peptides can be recognized by CTL.

Studies involving vaccination with autologous dendritic cells pulsed with Pr3 peptides, or liposomes containing the PR1 peptide or other Pr3-derived peptides are also contemplated in the present invention. In addition, the use of the HLA-A2,1-transgenic mouse model for the investigation into mechanisms of tolerance toward PR1, (Toes et al., 1996b; Toes et al., 1996c) are also contemplated. In addition, the PR1-tetramer could be used to select for a homogeneous population of leukemia-reactive CTL and may allow for the first time the adoptive transfer of CTL against MHC barriers to treat leukemia(Toes et al., 1996a).

### Example 9

### Antigen Presenting Cells (APC) Elicit Immunity Directed Against CML

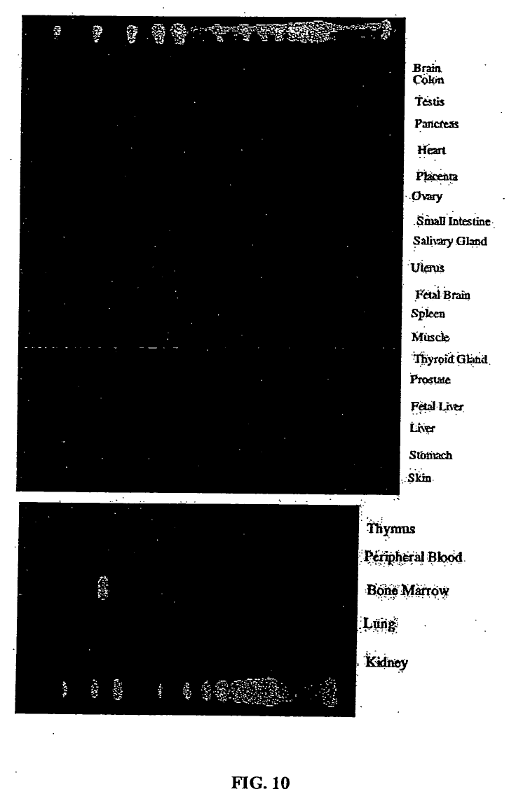

To study whether autologous antigen presenting cells (APC) derived from CML cells could elicit immunity directed against CML several approaches were investigated. First, it was determined whether autologous PR1-specific CTL could be elicited in vitro using autologous dendritic cells (DC) that were expanded from peripheral blood monocytes. Second, it was determined whether the elicited PR1-CTL demonstrate peptide-specific cytotoxicity. Third, it was determined whether PR1-CTL, that were elicited with autologous PR1-pulsed DC, could be further purified from bulk CTL cultures through the use of PR1/HLA-A2 coated microbeads and high speed flow cytometry. FIG. 10 shows that P3 transcripts were detectable only in bone marrow at 100× concentration (corresponding to approximately 100 pg of cDNA). Importantly, there is no P3 expression in tissue outside of the bone marrow, and in particular there is no thymic P3 expression, which suggests that peripheral tolerance may play an important role in controlling T cell immunity to PR1.

Analysis of PR1-specific immunity in myeloid leukemia patients after treatment with BMT or IFN, and deletional tolerance of PR1-specific T cells. In order to evaluate the relevance of PR1 as a leukemia antigen, it was determined whether PR1-specific CTL could be found in PBMC from HLA-A2.1+ CML patients undergoing different treatments using the PR1/HLA-A2 tetramer. PBMC from 38 HLA-A2.1+ CML patients at different stages of disease and 5 healthy volunteer donors were obtained and and cryopreserved. Diagnosis of CML was defined by 100% Ph+ cells in bone marrow aspirates from all patients prior to treatment. Ten patients received chemotherapy alone, including hydroxyurea, cytarabine, cyclophosphamide, and topotecan. Nineteen patients received IFN-a2b-based therapies, which consisted of IFN alone (6 patients) or combined with the chemotherapeutic drugs cytarabine and homoharingtonine (13 patients). Nine patients received an HLA-identical allogeneic BMT from a related donor. To assess the treatment response, metaphase cells from bone marrow aspirates were examined for the percentage of Ph+ cells. PBMC were simultaneously examined for PR1/HLA-A2 tetramer staining. Clinical responses were categorized as complete (CR, Ph, 0%), partial (PR, Ph, 1%-34%), or minor (MR, Ph, 35%-90%) (Faderl et al., 1999). Tetramer synthesis and validation was performed as previously described (Molldrem et al., 1999; Altman et al., 1996). The specificity of the PR1/HLA-A2 tetramer was demonstrated by its ability to stain a PR1-specific T cell line at 4° C. that was derived from a healthy HLA-A2.1+ donor, but not from a CMV-specific line derived from the same donor (data not shown).

None of the 10 patients that received chemotherapy alone without IFN and none of 5 HLA-A2.1+ healthy volunteer donor control samples had detectable PR1-specific CTL. Although one patient treated with chemotherapy alone had a response, no PR1-specific CTL were detectable (Table 11) by tetramer staining. Similarly, there were no detectable PR1-specific CTL in HLA-A2.1+ PBMC from three patients with multiple myeloma and two patients with newly diagnosed breast cancer that had received IFN.

TABLE 11
Patient Characteristics

| | Time from IFN | | |
|---|---|---|---|
| | withdrawal | Ph⁺ Cells by | |
| Patient | (months) | Cytogenet | bcr-abl⁺ by PCR |
| UPN1 | 18 | 0% | + |
| | 26 | 0% | + |
| UPN2 | 12 | 0% | + |
| | 18 | 0% | + |
| | 24 | 0% | + |
| UPN3 | 15 | 0% | + |
| | 21 | 0% | + |
| | 26 | 35% | + |
| | 32 | 0% | + |
| UPN4 | 45 | 0% | − |
| UPN5 | 76 | 0% | − |

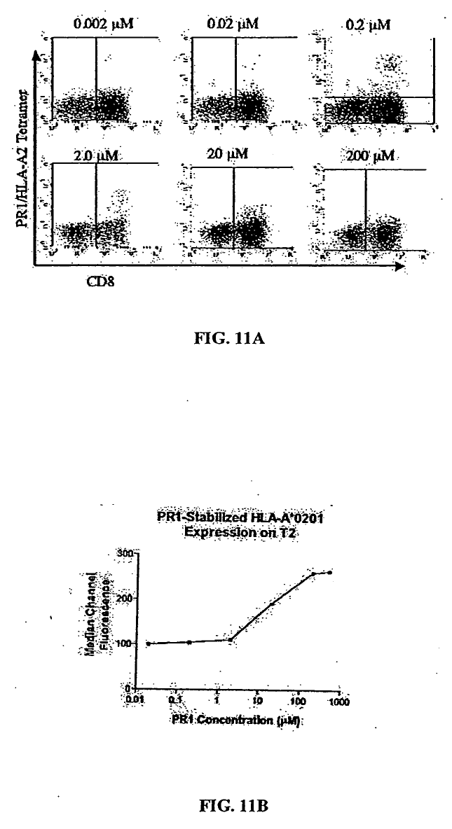

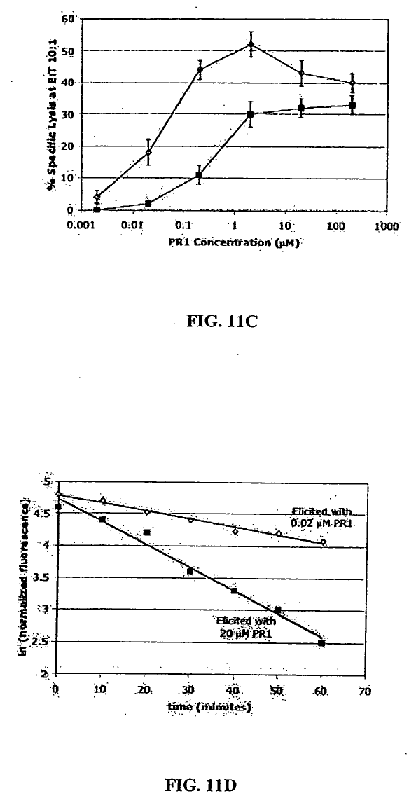

PR1-specific CTL with high and low TCR avidity can be elicited from healthy donors. To determine whether high and low avidity PR1-CTL are present in healthy donors, a modified tetramer staining technique (Savage et al., 1999) using limiting tetramer concentration to visualize high and low fluorescence intensity tetramer+ cells, which correlates with high and low TCR avidity was utilized. Sufficient PR1-CTL was elicited by stimulating PBMC with increasing peptide concentrations for 28 days. PBMC from an HLA-A2.1+ healthy donor stimulated weekly for 28 days with 0.2 μM PR1 elicited 1.3% high intensity PR1/HLA-A2 tetramer-staining CTL with a median channel of flourescence (MCF) 293 (high avidity PR1-CTL), while stimulation with 20 μM PR1 produced 3.1% low avidity (MCF=93) PR1-specific CTL (FIG. 11A). PBMC stimulated with 2 μM PR1 elicited PR1-CTL with intermediate TCR avidity (MCF=211). Total TCR-ab expression was comparable for cells elicited with 0.2, 2.0, or 20 μM PR1, suggesting that differences in tetramer staining were not due to differences in TCR expression level. Cultures stimulated with 2 μM PR1 produced fewer PR1-specific CTL with mixed TCR avidities, while stimulation with ≦0.02 μM PR1 induced <0.1% PR1-CTL. The PR1-CTL elicited with low (0.2 μM) PR1 (open circles) showed higher specific lysis of PR1-pulsed T2 target cells than PR1-CTL elicited with high (20 μM) PR1 concentration (closed circles), when both were normalized at an E:T of 10:1 based on the total number of PR1/HLA-A2 tetramer+events in the CTL cultures (FIG. 11C). To further verify that tetramer staining intensity correlates with TCR avidity, the kinetics of tetramer staining decay was determined using previously described techniques (Savage et al., 1999). PR1-CTL elicited with either low (0.2 μM) or high (20 μM) PR1 concentrations for 4 weeks were incubated with PR1/HLA-A2 tetramer and saturating concentration of BB7.2 anti-HLA-A2 monoclonal antibody to prevent rebinding. Normalized total fluorescence was measured at 4° C. at the appropriate time points and linear tetramer staining decay plots were obtained, indicating tetramer staining half-lives (t_{1/2}) should be proportional to the t_{1/2} of the respective TCR peptide/HLA-A2 complexes (FIG. 11D). PR1-CTL elicited with low (0.2 μM) PR1 showed a 3-fold longer t_{1/2} than PR1-CTL elicited with high (20 μM) PR1 (58 vs 19 min), which correlates with overall high and low tetramer fluorescence (FIG. 11A), respectively, and validates the use of overall tetramer fluorescence intensity to indicate relative TCR avidity.

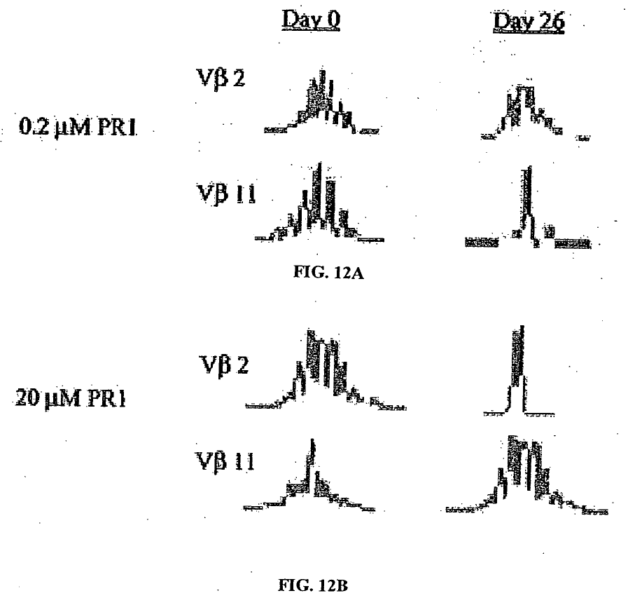

Spectratype from high and low avidity PR1-specific CTL cultures supports unique clonal derivation. To determine whether short-term PR1-CTL lines with high and low TCR avidities might be derived from distinct clonal populations, the TCR-Vβ CDR3 spectratype of 4-week old CTL from the same donor were analyzed. High avidity PR1-CTL elicited with 0.2 μM PR1 showed the most striking dominant clone to be present within TCR-Vβ family Vβ11, low avidity PR1-CTL elicited from the same donor with 20 μM PR1 showed a dominant clone in Vβ2 (FIG. 12). A Gaussian pattern was preserved in the majority of Vβ families in each of the PR1-CTL lines, with the exception of Vβ3, Vβ5.1 and Vβ14, which showed similarly skewed repertoires in the two different CTL lines. Similarly skewed spectratypes were obtained repeated experiments, suggesting unique clonal origin of high and low avidity PR1-CTL.

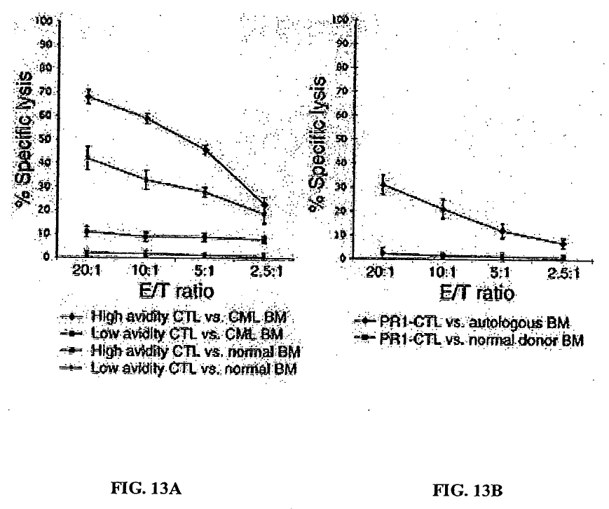

CML target cell killing by PR1-specific CTL correlates with TCR avidity. To evaluate whether CTL lines with different TCR avidities showed differences in effector function, 4-week old PR1-CTL lines derived from a healthy donor or from patients with CML were tested for the ability to kill HLA-A2+ CML target cells from a patient with blast crisis CML, autologous chronic phase CML cells from patient CML #3 at time of diagnosis, or cells from healthy donors. PR1-CTL derived from the healthy donor with high or low avidity were each combined with either bone marrow (BM) from the patient with CML or BM from the patient's healthy HLA matched sibling in a 4-hr cytotoxicity assay. High avidity PR1-CTL elicited with 0.2 μM PR1 showed nearly 2-fold greater lysis of the same CML BM cells on a per cell basis than did the low avidity PR1-CTL elicited with 20 μM PR1 (FIG. 13A). The specific lysis of autologous BM cells by a PR1-CTL line derived from a CML patient (CML #3) using 0.2 μM PR1 was similar to the amount of lysis of CML BM cells by the low avidity PR1-CTL line derived from the healthy donor (FIG. 13B). Similarly, CTL from patients CML #1 and CML #2 elicited with 0.2 μM PR1 showed lysis of CML #3 BM cells of 24%±5% and 33%±6%, respectively, at an E:T ratio of 20:1.

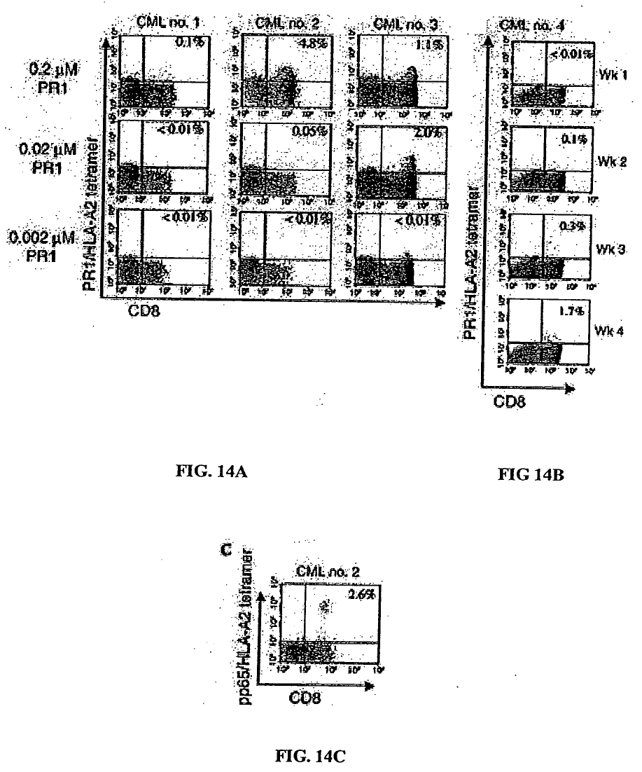

High avidity PR1-specific CTL are present only in interferon sensitive CML patients in cytogenetic remission. It was previously shown that detection of functional PR1-CTL in CML patients correlates with a cytogenetic response to interferon-α (Molldrem et al., 2000). This suggested that high avidity PR1-CTL would only be present in IFN-sensitive patients. To address this possibility PBMCs from untreated HLA-A2⁺ patients with either blast crisis (CML # 1) or chronic phase CML (CML # 2) or a patient with chronic phase treated with IFN-α for 3 months (CML # 3) were stimulated weekly with PR1. Only low-avidity PR1-CTLs could be elicited from any of the three patients (FIG. 14A). There were no detectable PR1-CTLs by tetramer staining in PBMCs prior to repeated peptide stimulation. Low-avidity PR1-CTLs were elicited with as little as 0.02 μM PR1 in patient 2 and 0.002 μM PR1 in patient 3, whereas PR1-CTLs from patient 1 could not be elicited below 0.2 μM PR1, and overall fewer CTLs were obtained. Patients 1 and 2 had 100% Philadelphia chromosome positive (Ph⁺) cells in the BM by karyotype, but patient 3 had developed a cytogenetic response to IFN-α and had 80% Ph⁺ cells. Because of the possibility that high-avidity CTLs may have arisen earlier during restimulation, cultures from a fourth untreated CML patient in chronic phase (CML no. 4) were studied weekly prior to restimulation with T2 cells pulsed with 0.2 μM PR1 for the emergence of tetramer-positive CTLs, and no high-avidity PR1-CTLs emerged during culture (FIG. 14B). Failure to elicit high-avidity CTLs was restricted to PR1, since high-avidity p65-specific CTLs from the CMV seropositive patient CML 2 was elicited using 0.2 μM pp65 peptide-pulsed T2 cells (FIG. 14C).

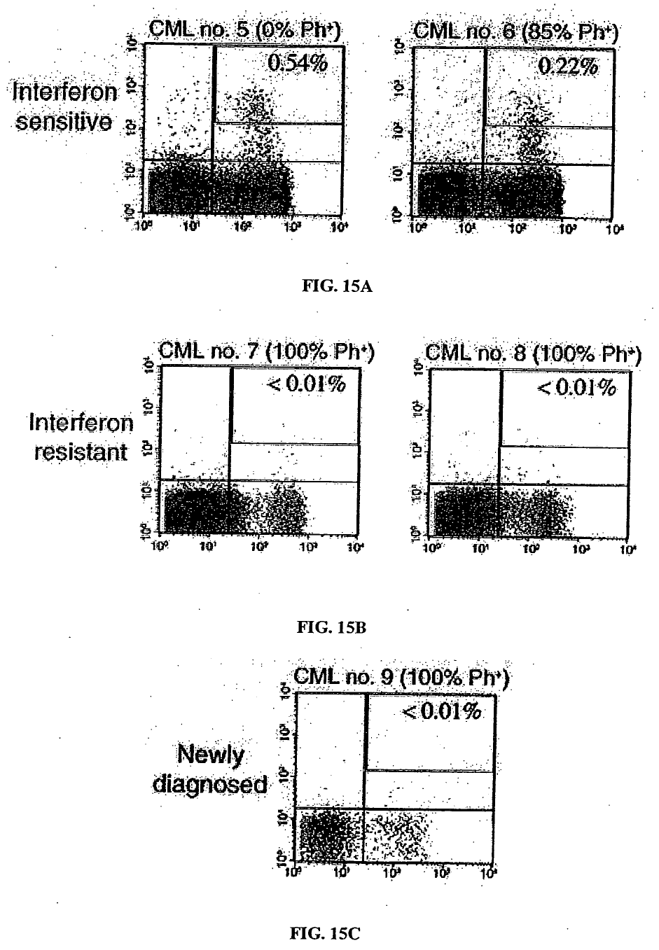

To further address the possibility that high avidity PR1-CTL would only be present in IFN-sensitive patients, two CML patients in a cytogenetic remission after 9 months of interferon treatment (CML #5 and CML #6 with 0% and 85% Ph+ cells, respectively), interferon resistant patients with no cytogenetic remission (CML #7 and CML #8), and one untreated newly diagnosed patient (CML #9) for the presence of PR1-CTL with high or low avidity TCR were studied. High avidity PR1-CTL were identified in both patients in a cytogenetic remission but in none of the interferon resistant or untreated patients (FIG. 15). However, low avidity PR1-CTL could be identified in the interferon resistant patients, but totaled less than 0.1% of CD8 cells. Furthermore, PBMC from untreated HLA-A2+ patients with either blast crisis (CML #1) or chronic phase CML (CML #2) or a patient with chronic phase treated with interferon-α for three months (CML #3) were stimulated weekly with PR1. Only low avidity PR1-CTL could be elicited from any of the three patients. This suggests that low numbers of high avidity PR1-CTL may be sufficient to contribute to cytogenetic remission in interferon sensitive patients, but leaves unanswered whether high numbers of low avidity PR1-CTL may contribute to remission.

High PR1 concentration and proteinase 3-overexpressing CML cells induce apoptosis of high avidity PR1-specific CTL. Previous studies showing that high affinity virus-specific T cells are eliminated by target cells infected with a high viral load (Alexander-Miller et al., 1998; Alexander-Miller et al., 1996a), and that CML cells frequently overexpressed proteinase 3 ((Molldrem et al., 1996; Molldrem et al., 1997), suggested that high avidity PR1-CTL might be undetectable in untreated CML patients due to selective elimination by CML cells that overexpress the target antigen. To demonstrate this, an equal number of PR1-CTL from a healthy donor were challenged with T2 cells pulsed with either high or low doses of PR1 and studied for evidence of apoptosis by Annexin V staining 16 to 18 hr later. High avidity PR1-CTL underwent apoptosis when challenged with high dose (20 μM) PR1 peptide, but not when challenged with low dose (0.2 μM) PR1 (FIG. 16). All high avidity PR1-CTL exposed to high dose PR1 were dead after 36 to 48 hr of co-culture. Apoptosis was abrogated in the presence of the BB7.2 blocking antibody to HLA-A2.1, and no apoptosis was observed when 20 μM of the irrelevant peptide Flu was used instead of PR1. In contrast, low avidity PR1-CTL did not undergo apoptosis when challenged with either high or low concentrations of PR1.

To determine whether CML cells similarly induced apoptosis of high avidity PR1-CTL, co-incubation studies were performed with HLA matched BM cells from CML patients followed by staining for Annexin V, for 16 to 18 hr after co-incubation. High avidity PR1-CTL underwent apoptosis by 18 hr after co-culture with BM from an HLA-matched patient with CML in chronic phase with 100% Ph+cells (FIG. 17A). No apoptosis was induced by co-incubation with BM cells from an HLA-A2 negative CML patient with 100% Ph+ cells, or by co-incubation with BM cells from an HLA-A2+ healthy donor. In contrast, less than 1% of the low avidity PR1-CTL underwent apoptosis when challenged with either the HLA-A2+ or HLA-A2− CML cells (FIG. 17B). Similar overexpression of cytoplasmic proteinase 3 was observed in BM cells from each of the CML patients (2.8-fold higher in the HLA-A2+ cells and 3.3-fold higher in the HLA-A2− cells compared to healthy donor BM cells), and MHC I expression was similar in the two patient samples (FIG. 17C). Therefore, differences in apoptosis were likely due to differences in the amount of PR1 peptide presented on the CML cells.

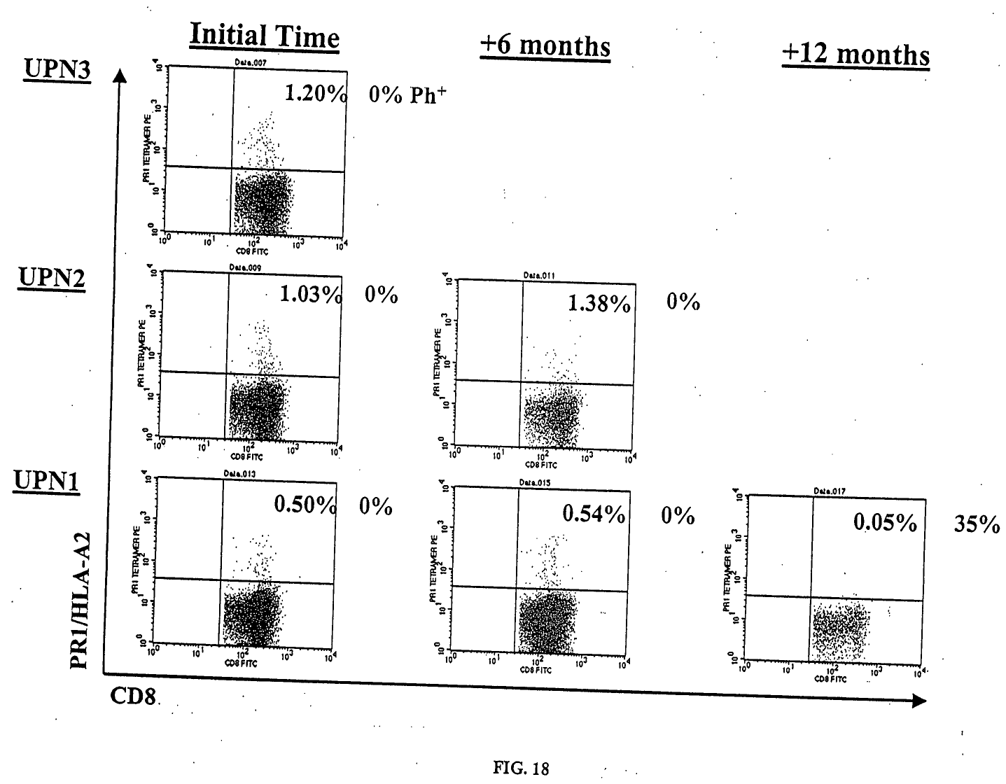

High avidity PR1-CTL persist in IFN-sensitive CML patients off of all therapy. Since it has been shown that IFN-sensitive patients have high avidity PR1-CTL in peripheral blood that can kill CML, it was determined whether PR1-CTL maintain remission in patients in CR off therapy. Three patients in complete cytogenetic remission after discontinued IFN were studied. The patients had CML from 5 to 9 years, and were off IFN from 18 to 26 months prior to study (Table 11 and Table 12). All patients continued to have bcr-abl transcripts by RT-PCR. Both high and low affinity PR1-CTL were identified in all patients, although only 25% to 30% of all PR1-CTL were of high affinity (FIG. 18). Importantly, all of the high affinity PR1-CTL were functionally active since stimulation with either PR1 peptide or SEB induced γ-IFN production by CFC and upregulated CD69, whereas the low affinity PR1-CTL did not produce γ-IFN, but did upregulate CD69 (not shown). Notably, the high affinity PR1—CTL from UPN3 upregulated CD69 but did not produce γ-IFN when tested at 21 months, indicating a loss of anti-leukemia immune function 5 months prior to cytogenetic relapse and the simultaneous disappearance of PR1 -CTL.

TABLE 12
PR1-CTL in IFN-Sensitive CML Patients off Therapy

| Patient | Length of | Time off IFN | % | % Ph Cells |
|---|---|---|---|---|
| (age, sex) | Disease (yrs) | (months) | PR1-CTL | (bone marrow |
| UPN1 | 6 | 26 | 0.74% | 0% |
| (61, female) | | | | |
| UPN2 | 5 | 12 | 0.96% | 0% |
| (42, female) | | 18 | 1.17% | 0% |
| UPN3 | 9 | 15 | 0.53% | 0% |
| (45, male) | | 21 | 0.19% | 0% |
| | | 26 | 0% | 35% |

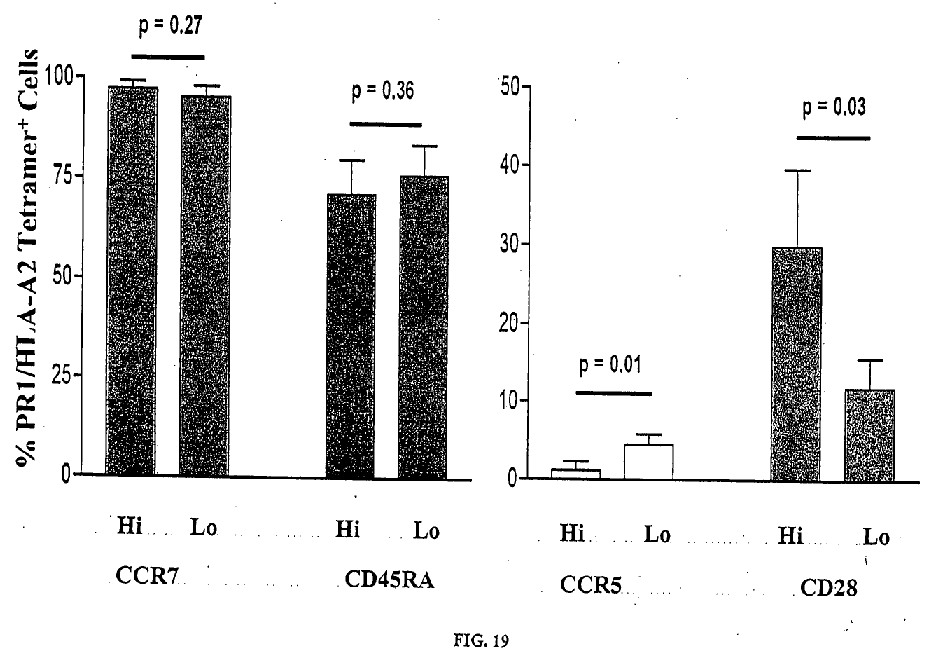

The only high affinity PR1-CTL were CD45RA+/CD28+/CCR7+/CCR5−, indicating an effector memory or possibly a naive phenotype. In addition, the high affinity PR1-CTL had significantly higher expression of CD28 (p=0.03) and lower expression of CCR5 (p=0.01) than low affinity PR1-CTL (FIG. 19), which suggested that low affinity PR1-CTL are terminally differentiated memory cells with little anti-leukemia activity. These results indicated that IFN treatment induces a long-lasting renewing population of high affinity PR1-CTL that continue to maintain lasting cytogenetic remissions in some patients after discontinuing IFN therapy. Loss of functional activity amongst high affinity PR1-CTL or the presence of only low affinity PR1-CTL suggests acquired tolerance, leading to eventual relapse. This early nonresponsiveness may reflect an anergic state, but loss of the PR1-CTL at the time of relapse may be due to deletion.

PR1 peptide vaccine can elicit PR1-CTL immunity in patients with myeloid leukemia. Preliminary data suggesed that in myeloid leukemia patients in whom a PR1-specific CTL immune response could be elicited or increased, PR1-CTL would convey an anti-leukemia immune response and contribute to remission. To test this in refractory leukemia patients, a phase I/II vaccine study was initiated. HLA-A2⁺ patients with CML (interferon-resistant or relapsed after BMT), AML (smoldering relapse or ≧2nd CR) or MDS (RAEB or RAEBt) with no detectable antibodies to proteinase 3 (no detectable cANCA) were eligible. Patients that relapsed after BMT or those ineligible for BMT were also eligible for study. Pregnant patients, HIV+ patients, and those with known vasculitis were excluded. Patients were required to have immunosuppression (i.e. cyclosporine, steroids) discontinued 4 weeks prior to study entry.

Primary endpoints were (1) toxicity assessment including the induction of autoimmunity resembling Wegener's granulomatosis, the systemic vasculitis associated with cANCA antibodies; and (2) induction of an immune response assessed by cytokine flow cytometry (CFC) of γ-IFN and PR1/HLA-A2 tetramer staining of PBMC before and 3 weeks after the last vaccination. Secondarily, clinical responses were assessed by standard criteria with bone marrow biopsy, cytogenetic studies (standard chromosome banding) and molecular studies (PCR for bcr-abl or other known abnormalities such as t(15;17), inv16, etc.) 3 weeks after the last vaccination. Patients were seen and evaluated in clinic every 3 weeks during the study period. Both vialed PR1 peptide (NSC698102) and the adjuvant (Montanide ISA-51, NSC675756) were used in this study.

The overall study was divided into two parts: Phase I consisted of nine patients treated in cohorts of three at 1 of 3 dose levels of 0.25 mg, 0.5 mg, or 1.0 mg of PR1 peptide given subcutaneously in incomplete Freund's adjuvant (Montanide ISA-51) and GM-CSF 75 mg subcutaneously every 3 weeks for 3 injections. The Phase II part of the study enrolled patients in cohorts of 4 randomized to one of the same three PR1 peptide doses since none of the doses were eliminated on the basis of toxicity during the Phase I part of the study. A continuous reassessment model was used in the statistical design to assess best dose level using criteria of immune response (>2-fold increase in the number of PR1-specific CTL during the vaccine study period) and grade 3 or 4 organ toxicity. If any patient developed vasculitis and/or cANCA, the trial would be stopped, and if any individual patient developed grade 3 or 4 organ toxicity the vaccine would be withheld for that patient. Any dose level would be discontinued if the number of patients with grade 3 or 4 toxicity, divided by the number of patients evaluated for toxicity, is greater than or equal to ¾, 4/8, 5/12 or 6/16. Any dose level would be terminated if none of the first 12 patients in that dose level have an immune response. If an immune response was noted, patients would continue to be entered onto that dose level, by continued randomization, to a maximum of 20 patients per dose level. If any clinical response was noted during the study period either with or without a measurable immune response, this would be considered an efficacy endpoint and patients would be entered onto that dose level to a maximum of 20 patients per dose level. Patients were monitored every 3 weeks with chest xrays, ANCA titers and physical examinations and for immune responses using PR1/HLA-A2 tetramers. Bone marrow cells (BMC) were obtained before the first vaccination and 3 weeks after the last vaccination to assess disease status.

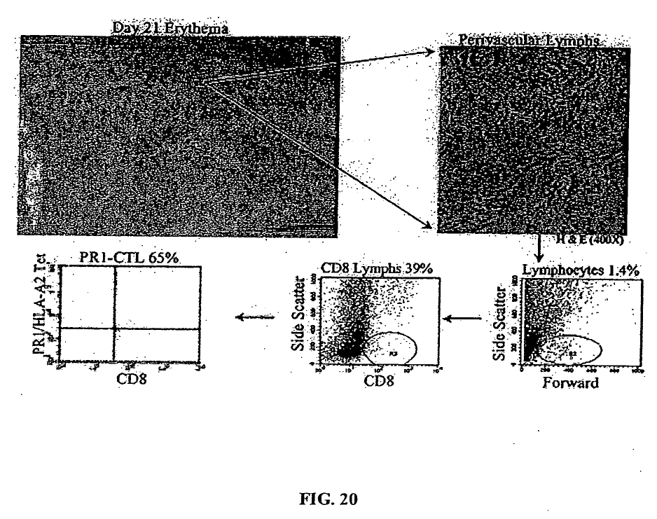

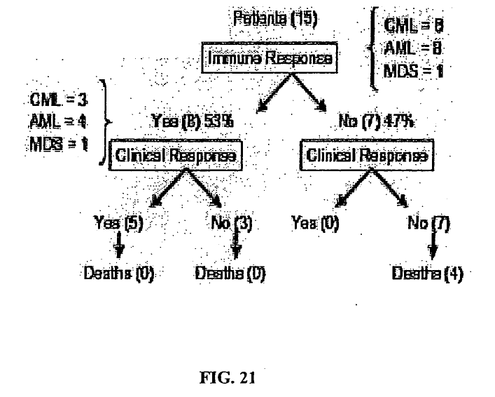

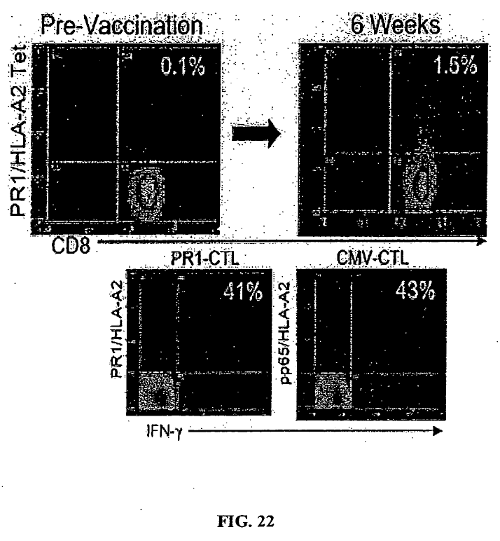

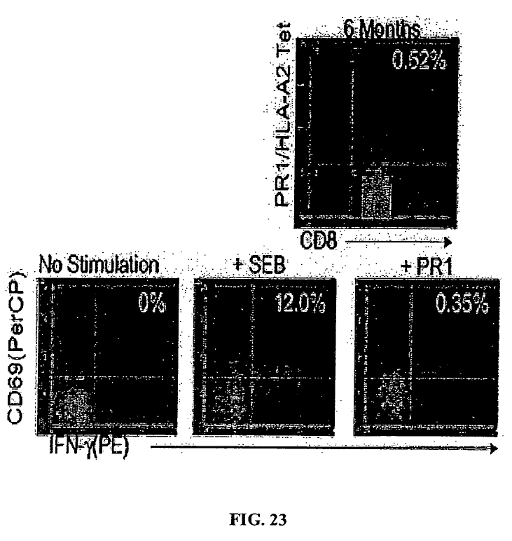

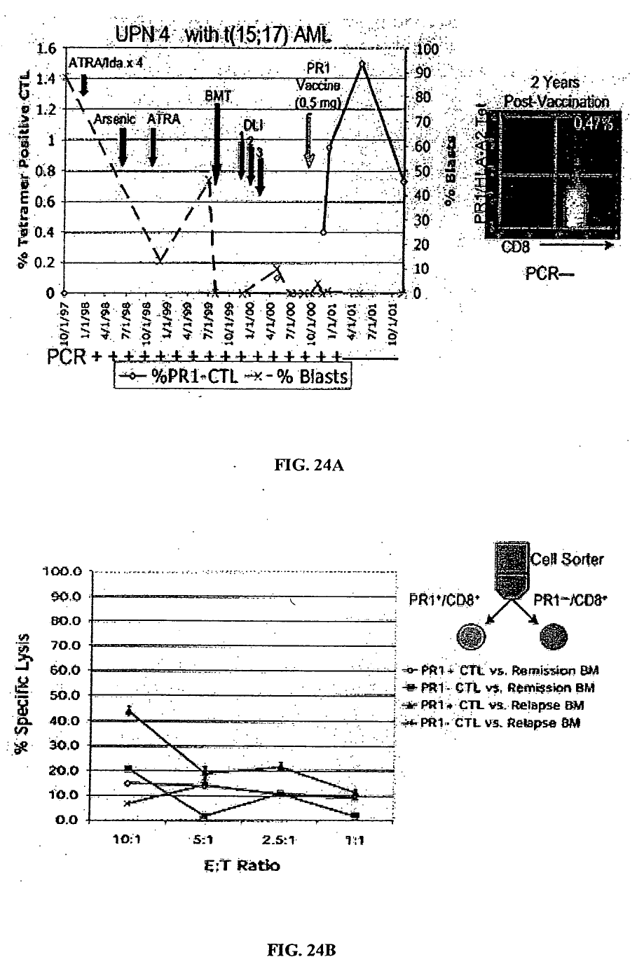

To date, 16 patients have been enrolled on the combined phase I/II study, and 15 patients are fully evaluable for toxicity, immune response and clinical response, as shown in FIG. 20. Two patients that had progressed on imatinib were kept on the same dose of imatinib to control blood counts and were treated with vaccine. One patient with AML was in hematological remission prior to study. None developed vasculitis or cANCA conversion (FIG. 20). A grade 2 cutaneous injection reaction was noted in one patient (UPN13, FIG. 20) at dose level 3 by two weeks after the first injection, which resolved after 1 week, and mild fatigue was noted in 4 patients. Skin biopsy from UNP13 showed a perivascular lymphocytic infiltrate and PR1/HLA-A2 tetramer staining confirmed that 65% of CD8+ lymphocytes were PR1-specific, demonstrating a localized immune response (FIG. 20). Immune response, defined as a >2-fold increase in the percentage of tetramer+ PR1-CTL, was noted in 8 of the 15 patients (FIG. 21). FIGS. 23 and 24 show that the elicited PR1-CTL were functional by CFC after incubation with 10 μM PR1 and that of the tetramer+ fraction, approximately half were able to secrete cytokine. Five of these eight patients were induced into CR during the study period, including 1 patient with overt acute leukemia. Although a secondary endpoint, clinical responses were also noted on this study (FIG. 21). Three patients (UPN4, UPN7 and UPN15) with relapsed AML prior to vaccination obtained cytogenetic remission (CR) after the second or third injection at dose levels 2, 3 and 1, respectively. They remained in CR at 20 months, 9 months and 4 months, respectively, with 0.58%, 0.2% and 1% circulating PR1-specific CTL (based on CD8 lymphocytes), respectively. Of the 6 patients with CML, only 2 had fewer than 100% Ph+ cells prior to study and one of these patients (UPN12), who had also progressed on imatinib, obtained cytogenetic remission (CR) on dose level 1 (0.25 mg). Because clinical responses were seen at all of the three dose levels, trial design obligates 20 patients on each dose level of the Phase II part of the study.

TABLE 13
PRI Vaccine Causes Minimal Toxicity
No induction of cANCA
No vasculitis or Wegener's-like autoimmunity
No maximum tolerated dose or dose limiting toxicity has been reached

| Toxicity | Grade | N |
|---|---|---|
| Fever | 1 | 3 |
| Local injection site reaction | 1 | 13 |
| | 2 | 1 |
| Injection site granuloma | | 2 |

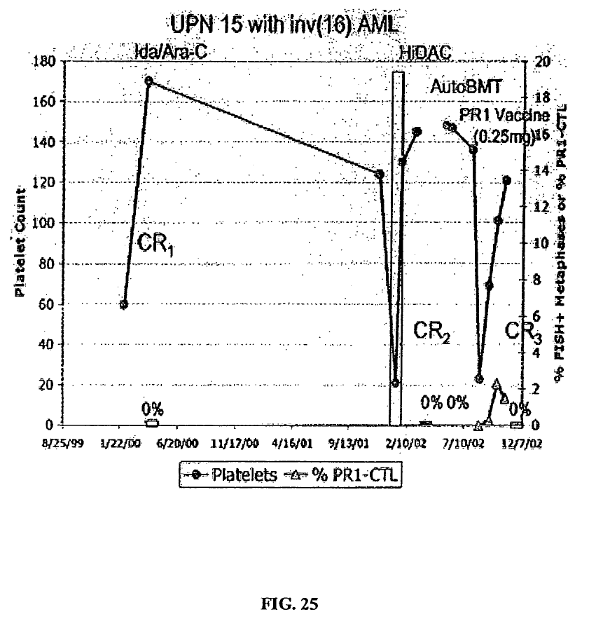

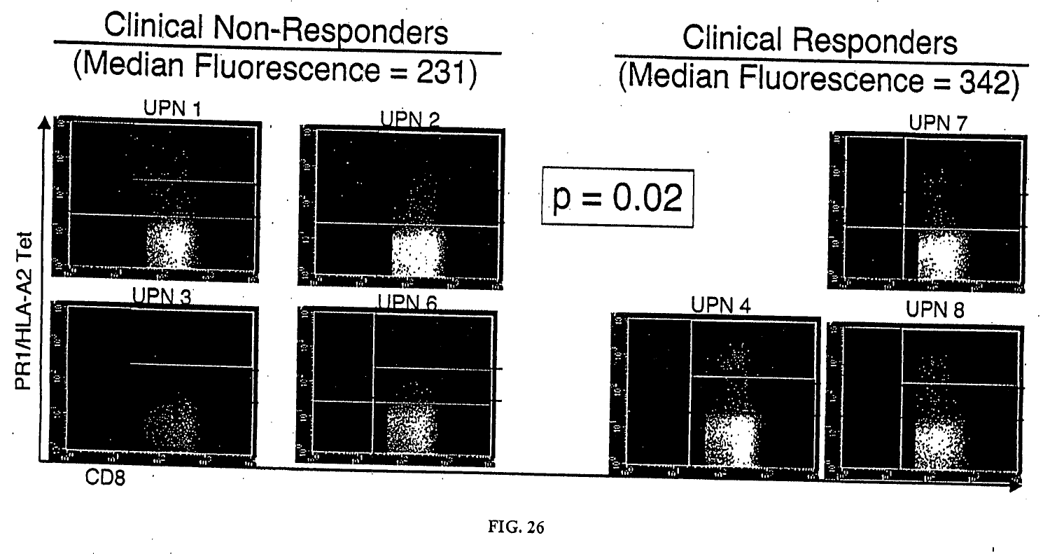

Vaccine-induced PR1-CTL contribute to cytogenetic remission. To show that PR1-CTL induced remission, cells were examined from UPN4, a 27-year-old man with APL in 4th relapse previously treated with allogeneic BMT and 3 escalating doses of DLI who separately consented to have PBMC collected by leukapheresis (LP) one month after the last vaccination. Donor-derived PR1-CTL increased from 0.1% to 0.54% after the 3rd vaccination, and were enriched for effector memory CD45RA+/CD28+/CD57− donor-derived CTL (by PCR microsatellite analysis), which correlated with loss of t(15;17) transcripts by RT-PCR 6 weeks after the first vaccination(FIG. 24A). PR1-CTL persist at 0.2% 22 months after completing the vaccinations. Tetramer sort-purified PR1-CTL obtained after vaccination showed PR1 specificity against peptide-pulsed T2 cells, and 44% lysis of the BMC collected at time of relapse versus only 14% lysis of BMC taken at time of remission, at E:T 10:1 (FIG. 24B). In another patient with invl6 μML (UPN15), PR1-CTL immunity was elicited at the time of 2nd relapse. This 32-year-old man received an autologous transplant while in 2nd remission and had no evidence of AML by FISH using probes for inv16. Two months later, his platelet count fell to 24,000 and he had inv16 by FISH (FIG. 25). By 3 weeks after the last injections of 0.25 mg (dose level 1) of the PR1 vaccine, his platelet count returned to normal and there was no inv16 by FISH or by RT-PCR. Taken together, data from this trial demonstrate for the first time direct evidence that peptide vaccination of leukemia patients can elicit highly active specific immunity against leukemia cells that induces cytogenetic remission. An immune response was necessary but not sufficient for a clinical response, although clinical responses correlated with the induction of an immune response (p=0.02). Thus, it was suggested that PR1-CTL would have higher TCR avidity in patients in CR since these CTL kill leukemia better than lower avidity PR1-CTL and would therefore show clinically effective antileukemia immunity. FIG. 26 shows that by using low doses of tetramer to identify high and low avidity PR1-CTL, it was found that the average TCR avidity of PR1-CTLs was 1.5-fold higher in the clinical responders compared to post-vaccination PR1-CTL in the non-responders that had pre-existing tetramer+CTL (p=0.02).

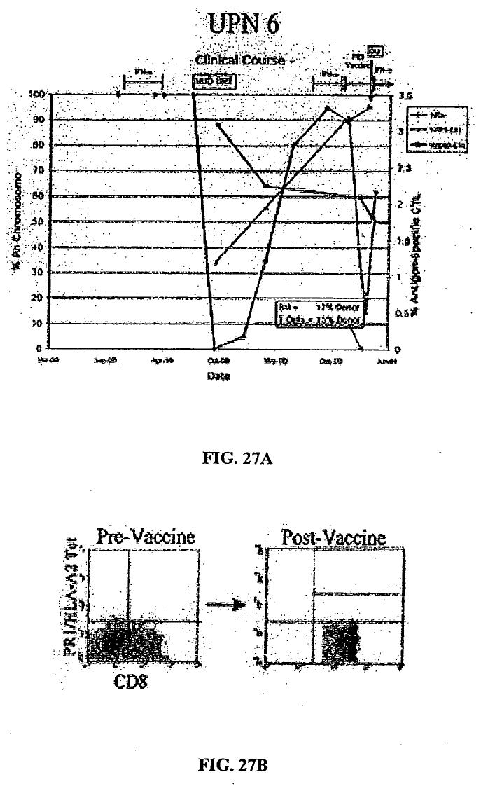

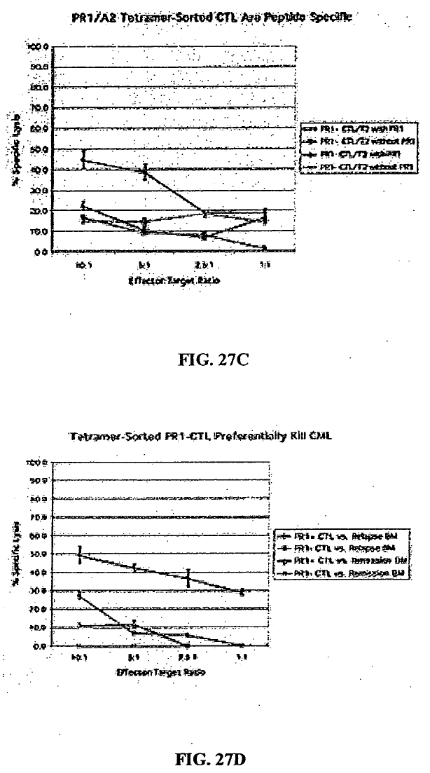

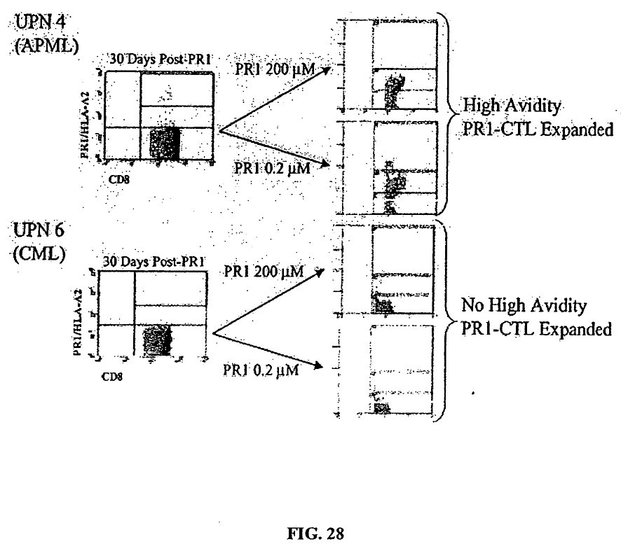

T cell tolerance from loss of high avidity PR1-CTL and downregulation of P3 antigen correlates with progression of CML after vaccination. UPN6, a non-responder, was found to have both high and low avidity PR1-CTL prior to vaccination with 0.5 mg of PR1, but only low avidity PR1-CTL 3 weeks after the last injection (FIG. 27). Furthermore, the overall number of PR1-CTL decreased from 3.2% to 0.5% during the study and the number of Ph+ metaphase chromosomes increased from 90% to 95% (FIG. 27). Interestingly, PR1/HLA-A2 tetramer-sorted CTL showed peptide-specific lysis of T2 cells pulsed with 20 μM PR1 that was similar to PR1-CTL from UPN4 that produced nearly equivalent lysis (on a per cell basis) of T2 cells pulsed with only 2 μM PR1. Although UPN6 received the same peptide dose as the responder UPN4 during vaccination, this dose was sufficient to eliminate the high avidity PR1-CTL and allow outgrowth of CML. Since high avidity PR1-CTL were not detectable in peripheral blood PBMC in UPN6, PR1-pulsed T2 cells were used to determine whether they could be expanded. PBMC from LP products from both UPN4 and UPN6 were stimulated weekly with varying doses of PR1 (0.2 μM to 200 μM) coated T2 cells. However, as shown in FIG. 28, high avidity PR1 -CTL could only be expanded from UPN4, but not from UPN6. This experiment supported the expectation that high avidity PR1-CTL were deleted in this non-responder. However, high avidity PR1-CTL may still be present in UPN6 but cannot be expanded, perhaps due to poor proliferative potential, poor cytokine production, or a more generalized antigen nonresponsiveness.

Since it has been well described that defective antigen processing (Restifo et al., 1993; Maeurer et al., 1996), target antigen mutation or downregulation of MHC (Lehmann et al., 1995) or target antigen (Marincola et al., 1996) can lead to immune escape and tumor outgrowth, studies were conducted to investigate these possibilities by comparing P3 expression before and after vaccination in both UPN4 and UPN6. It was found that P3 decreased by 60% in UPN6 after vaccination, compared with no change in P3 expression in UPN4. Interestingly, P3 expression shifted to a more immature CD34+ progenitor cell, which have lower MHC expression (Bernhard et al., 1995) and may be less able to process and present antigen to T cells than more mature myeloid cells in the marrow. Together, these studies show preferential loss of high avidity PR1-CTL with a simultaneous decrease in P3 target antigen expression in the leukemia cells in UPN6, a clinical non-responder, compared to an expansion of high avidity PR1-CTL and preserved overall P3 expression after vaccination in UPN4, a clinical responder. Overall MHC-I, CD80 (B7.1) and CD54 (ICAM-1) expression remained unchanged in both patients before and after vaccination. Therefore, at least two or more mechanisms appear to be operative in UPN6 leading to eventual escape from anti-leukemia immunity.

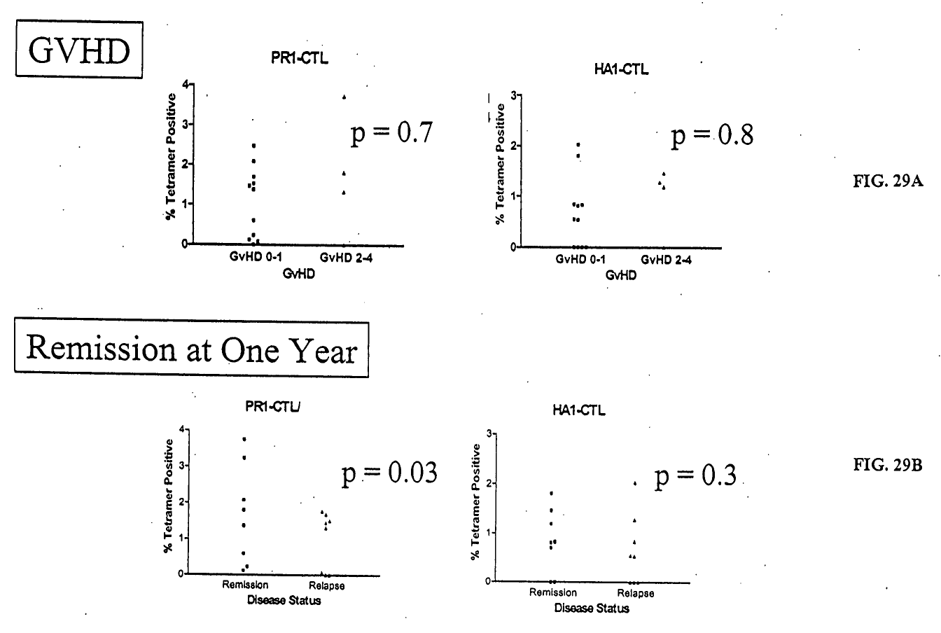

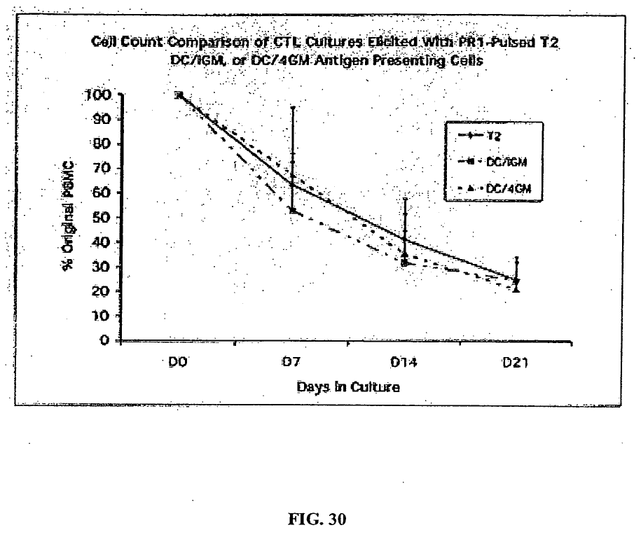

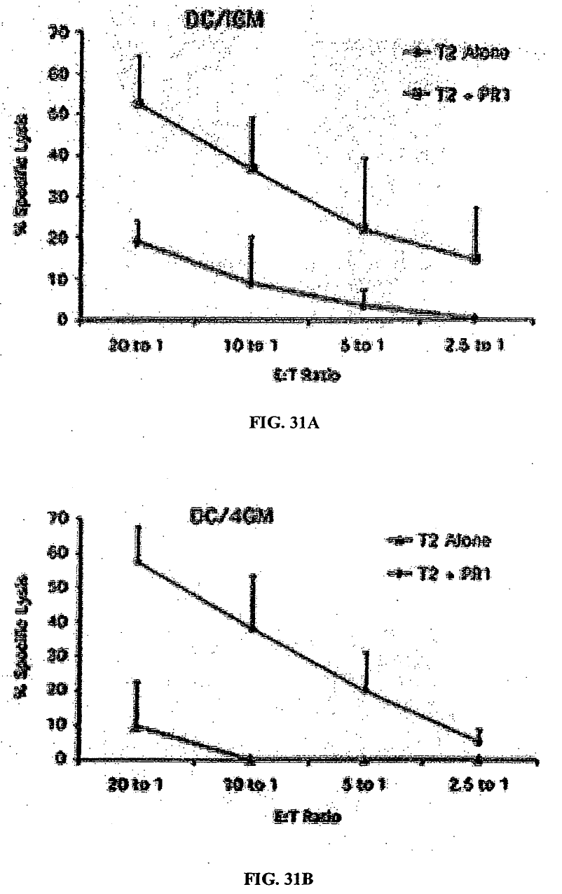

Monocyte-derived dendritic cells (DC) can expand autologous PR1-specific CTL and PR1/HLA-A2 monomers can be used to select PR1-CTL for adoptive immunotherapy. Another central aspect of this invention is that CTL that contribute to GVL and those that cause GVHD after allogeneic bone marrow transplantation (BMT) target unique antigens. Previously, the inventors identified the HLA-A2-restricted peptide PR1, derived from P3, that can elicit CTL that preferentially kill CML over normal bone marrow. Furthermore, PR1-specific CTL could be identified in the peripheral blood of BMT recipients that were in cytogenetic remission using soluble peptide/MHC tetramers to stain peripheral blood mononuclear cells. More recently, the inventors have studied the peripheral blood of AML patients that received of nonmyeloablative stem cell transplant (NST) regimens for evidence of PR1-CTL. Patients were studied around day 60 post-NST using HLA-A2 tetramers folded with different peptide epitopes to determine whether immunity directed against minor histocompatibility antigens such as HA-1 coexisted with immunity against self peptides such as PR1. Using combinations of the fluorochromes PE(Cy7) and PE, tetramers were constructed that can be used to simultaneously stain small aliquots of peripheral blood for different peptide-specific T cells using multiple tetramers, which produces results that are similar to single tetramer-stained blood samples (FIG. 29A). It was found that around day 60 a higher percentage of CD8⁺ T cells specific for PR1 correlated with remission at one year post-NST (p =0.03, FIG. 29B). By combining tetramers for PR1, HA-1 and WT-1, a dominance hierarchy can be defined, and the relative contribution of immune responses against each antigen toward GVL and GVHD determined. Furthermore, it was reasoned that these reagents might also be used to select for peptide antigen-specific CTL amongst polyclonal T cells that could be used in adoptive immunotherapy. Since the precursor frequencies of these T cells is very-low, especially for self-antigens, selection with tetramer staining would need to follow the preferential expansion of the antigen-specific T cells in bulk culture. A clinically practical method of expanding peptide-specific CTL ex vivo for use in adoptive transfer to patients after BMT was sought. Peptide-pulsed T2 cells (a highly reliable but unsuitable clinical method for eliciting peptide-specific CTL) were compared with peptide-pulsed dendritic cells (DC). Because it is desirable to use reagents that are already approved for clinical use, DC expanded from peripheral blood monocytes were employed using both interferon-α2b (IFN) and GM-CSF, as these cells would be useful as peptide-pulsed APC to expand antigen-specific T cells (Santini et al., 2000). DC expanded for 7 days were compared using IFN (1,000 U/ml) and GM-CSF (500 ng/ml) versus DC expanded for 7 days with IL-4 and GM-CSF. The yields of PR1-specific CTL from autologous PBMC were compared using each of these APCs from 7 healthy HLA-A2+ donors (FIG. 30). PBMC from each of the patients were stimulated weekly with PR1 peptide-pulsed T2, IFN+GM-CSF-treated DC (DC/IGM) or IL-4+GM-CSF-treated DC (DC/4GM). As shown in FIG. 30, the yields of peptide-specific CTL at the end of 21 days in culture were similar amongst all three groups. Cytotoxicity experiments also confirmed peptide specificity and a comparable amount of specific lysis of PR1-pulsed T2 cells as targets amongst all three groups (FIG. 31). DC/IGM are been pursued as reliable sources of APC that can be used to elicit PR1-specific CTL for the adoptive transfer studies. In addition, the experiments shown in FIGS. 24B and 27 demonstrated the feasibility of using tetramers which can be used to select PR1 -specific T cells by high-speed flow cytometry, if the PR1-CTL are present at sufficiently high frequency. Importantly, the PR1-CTL retained their cytotoxic potential against leukemia after sorting.

### Example 10

### Specific Immunity Induced after PR1 Peptide Vaccination Correlates with Cytogenetic Remission

The inventors have shown that patients with CML that have a cytogenetic response to IFN-γ or that are in remission after allogeneic BMT have circulating cytotoxic T lymphocytes (CTL) with specificity for the HLA-A2 restricted peptide PR1. The PR1-CTL show peptide-specific lysis of leukemia and are long-lived in some patients. Although this shows that PR1-CTL may be important in clearing the malignant cells, it is not clear whether PR1-CTL developed in response to the malignancy and are sufficient for the elimination of it. In a phase I study evaluating escalating doses of PR1 peptide administered subcutaneously combined with incomplete Freund's adjuvant and GM-CSF, the results were reported from two patients that received 3 injections of 0.5 mg PR1 every 3 weeks: a 38-year-old woman with late chronic phase CML relapsed after allogeneic BMT (UPN6) and a 27 year old man with refractory APML after allogeneic BMT and DLI×3 (UPN4). PR1/HLA-A2 tetramers were used to identify PR1-CTL and to FACsort them to >99% purity from leukapheresis products obtained at the end of the trial period for functional analysis. Chimerism of the total T-cell population and of FACsorted PR1-CTL was performed by DNA microsatellite analysis. Until 1 month prior to the vaccine, UPN6 received IFN-γ and had 1.9% PR1-CTL, which declined to 0.83% 1 month after the 3^{rd} vaccination. Although there was only 15% overall donor T-cell chimerism, the PR1-CTL were 100% donor-derived. This patient had no clinical response with persistence of 95% Ph⁺ cells. In contrast, UPN4 had <0.1% PR1-CTL prior to vaccine, which increased to 1.5% after the 3^{rd} vaccination and remained 100% donor T-cell chimeric throughout. At the same time, detectable t(15;17) transcripts by RT-PCR prior to vaccination were no longer detectable 1 month after vaccination. Interestingly, UPN6 developed limited oral and ocular chronic GvHD 1 month after receiving the PR1 vaccine. The functional capacity of FACsorted PR1-CTL to CD8⁺ PR1/HLA-A2 tetramer negative cells was then compared in a standard 4-hr cytotoxicity assay. PR1-CTL from both patients showed PR1 peptide specificity using PR1-pulsed T2 cells as target cells at E:T ratios from 10:1 to 1:1. PR1 -CTL from UPN4 showed 49% specific lysis of CML bone marrow (BM) cells obtained prior to BMT compared to only 27% specific lysis by the tetramer-CTL at E:T 10:1. Likewise, PR1-CTL from UPN4 showed 44% lysis of BM at time of relapse versus only 14% lysis of BM taken at time of remission. By contrast, the CD8⁺ PR1/HLA-A2 tetramer negative cells showed only 25% and 6% lysis of relapse and remission BM, respectively, at E:T 10:1. This study demonstrated for the first time direct evidence that peptide vaccination of a leukemia patient can induce highly active specific immunity against the leukemia cells. Further studies are been conducted to investigate both the PR1-CTL and remaining target cells from each of the patients to determine why vaccination in UPN6 lead to a decline in the number of functional PR1-CTL. It is possible that APML is more susceptible vaccine target since expression of proteinase 3, the protein from which PR1 is derived, is more abundantly expressed compared to CML cells.

To determine an optimal method to expand PR1-specific cytotoxic T lymphocytes (CTL) with high avidity T cell receptors ex vivo. In this study, whether a combination of IFN and GM-CSF can produce DC that can be used to elicit PR1-CTL for adoptive immunotherapy was tested. Based on previous observations that PR1-CTL preferentially kill leukemia over normal BM due to P3 overexpression, and because P3 is only expressed in hematopoietic tissue, it was indicated that adoptively transferred PR1-CTL will produce GVL without GVHD. It has also been shown that high avidity PR1-CTL are more efficient killers of leukemia cells, although they may also undergo apoptosis when incubated with highly P3-expressing leukemia cells. This further suggested that if large numbers of high avidity PR1-CTL were adoptively transferred to myeloid leukemia patients with minimal disease after allogeneic bone marrow transplant, a sufficient GVL effect would be produced before significant leukemia-induced loss of the PR1-CTL resulted, which would facilitate remission without significant GVHD. Two general expansion methods to elicit PR1-CTL in vitro will be compared as previously described.

In brief, DCs were grown using combinations of either 1,000 U/ml IL-4 plus 500 U/ml GM-CSF (termed DC/4GM) or 1,000 U/ml interferon-α2b plus 500 U/ml GM-CSF (termed DC/IGM). Previously cryopreserved PBMC from HLA-A2+ healthy donors were thawed, washed and adhered to plastic flasks prior to the addition of media+10% human serum (HS) with the addition of the above cytokines. T2 cells were maintained in RPMI+10% HS prior to co-culture with donor PBMC. At the end of 7 days, DC were pulsed with 20 μg/ml PR1 peptide, irradiated and combined with fresh PBMC from the same donor at a 1:2 ratio. On day 7, the culture was restimulated with PR1-pulsed DC (or T2) and on day 8 IL-2 at 20 U/ml was added to the cultures. Restimulation and IL-2 addition was repeated weekly until day 26 through 28 when the PR1-CTL cultures were tested for their ability to lyse PR1-coated target cells or CML cells. The PR1-CTL was evaluated for surface phenotype with the PR1/HLA-A2 tetramer and anti-CD8. In general, both DC/IGM and T2 cells elicited PR1-CTL. However, T2 were nearly twice as efficient, typically yielding 3% to 6% PR1-CTL (of the bulk culture, determined by tetramer staining) by 4 weeks and DC/IGM yielding only 1.5% to 4% PR1-CTL. Because of the regulatory concerns using T2 cells in the clinical setting, alternative sources of stimulators will be studied.

To further improve the efficiency of CTL expansion, artificial antigen presenting cells (AAPC) were constructed as a substitute for DC. The advantage of this approach is that it is more readily available and there is potentially more consistency in the final synthetic product. In preliminary experiments using the PR1 peptide, HLA-A*0201 heavy chain and β2-microglobulin were each produced in E. coli and the expressed protein was folded in vitro in the presence of PR1. Folded monomeric complexes were purified by gel filtration and ion exchange. The PR1/HLA-A2 monomers were then mixed with dioleolyl phosphatidylcholine (DOPC) (Avanti Polar Lipids), at molar ratios of 1:100, 1:500 or 1:1000, frozen in dry ice/acetone bath, and lyophilized overnight to remove organic solvent. The lyophilized product was stored at -20° C. and then hydrated in PBS buffer and vortexed vigorously before use. T2 cells were incubated with PR1/HLA-A2 AAPC (1:100, 1:500, and 1:100 molar ratio) for 3 hr and 24 hrs. After incubation, the cells were surface stained with murine anti-HLA-A*0201 monoclonal antibody BB7.2 and secondarily stained with FITC-labeled goat anti-mouse antibody, and observed under the confocal fluorescent microscope to determine proper insertion of PR1/HLA-A2 into the artificial membrane. After this was confirmed, the efficiency of liposome-encapsulated T2 cells were compared to DC for eliciting PR1-CTL after 4 weeks in culture with weekly stimulation. CTLs elicited with PR1/HLA-A2 AAPC for 3 to 5 weeks efficiently lysed T2 target cells pulsed with PR1 peptide and was equivalent to lysis by DC-generated PR1-CTL lines. The number of PR1-CTL produced with either method was equivalent in the initial experiment, but in two additional experiments the AAPC appeared to be similar to T2 cells, which generally are more efficient than DCs at eliciting peptide-specific CTL, in their ability to expand PR1-CTL.

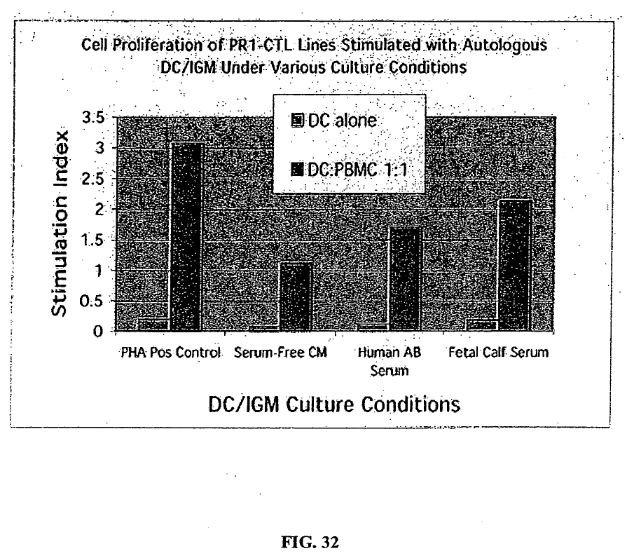

In preliminary experiments, it was noted that antigen expression could be increased at higher molar ratios of peptide/A2 monomer to DOPC, which may improve the efficiency further. However, apoptosis of high avidity PR1-CTL may occur as a consequence, and it is likely that a “therapeutic window” of antigen concentration will be optimal. The DC/IGM will be directly compared to DC grown using IL-4, GM-CSF and TNF-α, and to both AAPC and peptide-pulsed T2 cells. As discussed above, in preliminary experiments using PR1 it was shown that AAPC are equivalent to T2 cells, which are in turn more efficient than DC/IGM. Typical yields of antigen-specific tetramer+ cell numbers are 3% to 5% PR1-CTL using AAPC or T2 cells, but only 0.3% to 2% when DC/IGM are used. These observations are likely to apply to HA-1 and other putative target antigens of GVL (WT-1), but all of the comparative experiments will be repeated using these peptides. In addition, serum-free growth conditions will be compared to 10% HS and 5% albumin as a serum substitute in both the DC cultures and the CTL cultures. In preliminary experiments, pooled human AB serum was nearly equivalent to FBS as a supplement to media used to expand DC/IGM that could then be used as peptide-pulsed APC to expand PR1-CTL (FIG. 32). Once these methods have been confirmed, scaleup procedures will be carried out in both flasks and closed bag systems to demonstrate the feasibility for clinical use. A particular aspect of this study is to first determine the conditions that produce the highest numbers of functional high avidity PR1-CTL in the shortest period of time. Once conditions are optimized and the scale-up experiments show feasibility, an adoptive immunotherapy study will begin using PR1-CTL generated ex vivo. Weekly tetramer staining during bulk culture restimulations will be used to compare yields and avidity of PR1-CTL, and CFC and cytotoxicity experiments will be used to compare the effector function of the cells.

Secondly, two methods to select antigen-specific CTL from bulk culture will be compared. Although it is suggested that the bulk culture conditions described above will yield CTL that, when adoptively transferred to patients, will not produce significant GVHD, it is possible that GVHD may occur due to the non-specific CTL remaining in the bulk cultures. Since CTL cloning at the end bulk culture is time-consuming and would therefore need to be performed prophylactically in each patient, and because the transfer of CTL clones is not likely to yield long-lived CTL in vivo (Riddell et al., 1997), separation of the peptide-specific fraction from the bulk culture will improve the purity of PR1-CTL and thereby reduce GVHD. Initial cell transfer will involve escalating doses of bulk cultures of peptide-expanded CTL carried out in previous experiments. If there is no GVHD, then the dose of non-selected bulk culture PR1-CTL will be escalated. If patients develop >grade II GVHD, then PR1-CTL will be selected from the bulk cultures using methods described below. In the first approach to select the antigen-specific CTL, the use of high-speed flow cytometry will verify separate antigen-specific CTLs using soluble peptide/MHC tetramers. By staining leukeapheresis products with anti-CD8 and PR1/HLA-A2 tetramers, PR1-CTL can be separated from the remainder of CD8+ lymphocytes, and these sorted cells can efficiently kill leukemia cells (FIGS. 24B and 27).

Preliminary experiments were designed to obtain 1×10⁶ PR1-CTL for the cytotoxicity experiments, however, this could be scaled up to obtain sufficient cells for adoptive immunotherapy from a cell product that contained as few as 0.4% PR1-CTL (based on CD8+ lymphocytes). For instance, 10¹⁰ total cells collected from leukapheresis would be expected to yield 2×10⁹ cells at the end of a typical 4-week expansion. Based on minimum assumptions about the yield of cells at the end of the 4-week expansion, including cells where 40% are lymphocytes with only 20% of those as CD8 lymphocytes and a tetramer frequency of only 0.4% PR1-CTL, a dose of 0.9×10⁶ PR1-CTL/kg could be adoptively transferred to the average 70 kg recipient. The cell purity was >90% and the efficiency was >95%.

In the second approach, the IFN capture method will be compared to a modified peptide/MHC-conjugated bead method developed to select antigen-specific CTL. In preliminary experiments using the PR1 peptide, the feasibility of using PR1 peptide/HLA-A2 monomers linked to streptavidin-coated microbeads (Miltenyi Inc, Germany) to separate antigen-specific CTL from bulk cultures of mixed lymphocytes was demonstrated (Wang et al., 2000). Short-term polyclonal CTL lines were elicited by pulsing 70×10⁶ PBMC from 6 different healthy HLA-A2.1+donors weekly with PR1-coated T2 cells plus interleukin-2. After 21 days in culture, these PR1-pulsed CTL, quantified with a PR1-HLA-A2 tetramer conjugated to phycoerythrin (PE), comprised 2% to 8% of the culture. To preferentially select PR1 antigen-specific T-cells, a method using streptavidin-coated microbeads (Miltenyi, Inc.) was developed and optimized. Beads were conjugated with HLA-A2.1 heavy chain plus PR1 myeloid leukemia peptide via a biotin-labeled C-terminus of the heavy chain. PR1-pulsed short-term CTL lines were incubated for 30 min with PR1/HLA-A2 coated microbeads and were selected using the magnetic MACS column. Bead-selected and non-selected CTL were quantified by FACS analysis using the PR1-HLAA2 tetramer and compared to the initial polyclonal CTL lines. Yields ranged from 85% to 100%, and PR1-specific CTL purity ranged from 11% to 21% after selection (3- to 5-fold increase in purity of antigen-specific CTL). Specific lysis of both PR1-coated T2 cells and CML marrow cells increased in all 6 cultures and background lysis of normal HLA-A2.1 normal marrow was eliminated compared to non-selected PR1-specific CTL. Both the flow cytometry method and monomer-coated microbead method will be compared to the cytokine capture microbead technology developed by Miltenyi Inc., which relies on the ability of antigen-specific CTL to secrete IFN after antigen challenge. PR1-CTL obtained after 4 weeks of weekly restimulation will be incubated with PR1 antigen and the bi-specific antibody with anti-CD45 and anti-IFN binding will be co-incubated with the cells. A secondary antibody with anti-IFN antibody that is directly linked to microbeads (supplied by Miltenyi, Inc) will then be used to select out IFN-secreting CTL from bulk culture.

Although it is reasonable to expect that the commercial cytokine-capture technology will produce sufficient purity of CTL, the device does not capture all CTL with the potential to recognize the cognate peptide/MHC ligand. Since it is unclear whether non-secreting antigen-specific CTL might be required to maintain a more functional fraction in vivo, or whether the non-secreting CTL later develop the potential to express effector function, and may be selecting too few CTL using the commercial product. Similarly, monomer-coated beads have the potential to be more easily scalable on a molar ratio basis so that high avidity PR1-CTL can be selectively purified.

To determine whether adoptively transferred proteinase 3 peptide-specific CTL contribute to the graft-versus-leukemia (GVL) effect. Another aspect of the invention is to enhance GVL and reduce GVHD by using a preparative regimen to achieve engraftment of transplanted allogeneic blood stem cell or bone marrow, which can then be used as a platform to deliver antigen-specific CTL therapy. It was postulated that GVHD could be eliminated and GVL enhanced by adoptively transferring antigen-specific T cells with preferential GVL activity. Therefore, whether adoptive transfer of high avidity PR1-CTL induces GVL without increasing GVHD will be tested, the clinical trial will determine the maximal dose of PR1 -specific CTL that will provide GVL while preventing the development of acute GVHD (the baseline rate of grade 3 and 4 acute GVHD <15%). T cells have been shown to cause GVHD (Drobyski et al., 1994; Mavroudis et al., 1996; Gaschet et al., 1996; Debergie et al., 1997; van Lochem et al., 1992; Barrett et al., 1998), facilitate engraftment (Reich-Zeliger et al., 2000) and are required to prevent rejection with established nonablative preparative regimens developed at M.D. Anderson Cancer Center (Houston, Tex.) and elsewhere. Leukapheresis samples from healthy HLA-matched donors will be used to expand PR1-CTL ex vivo, using the expansion methods outlined herein. The expansion methods developed in this invention will also be used for the production of cellular products for clinical use. Patients with advanced disease, who are eligible for the transplant studies, will receive allogeneic BMT and imatinib mesylate after transplant and will be monitored by PCR for evidence of persistent or increasing disease. If patients that are HLA-A2+ remain PCR+ for bcr-abl at day 60, then patients will receive PR1-CTL expanded ex vivo in escalating doses. By skewing the ex vivo culture conditions with low doses of PR1 as shown in the preliminary results, high avidity PR1-CTL will preferentially be expanded from the donors. These cells will be infused into PCR+ donors after 4 weeks of expansion at escalating doses starting at 1×10⁷, 5×10⁷ and 1×10⁸ bulk CTL/kg of recipient weight. At 4 weeks of culture, the bulk PR1-CTL cultures contain from 1.5% to 4% PR1-specific CTL, as observed, by tetramer staining and functional analyses, and based on expansions perviously performed. Two leukapheresis products (LP) will be required to (1) elicit sufficient DC/IGM as APC (from cryopreserved aliquots of LP), which will be pulsed with PR1 peptide at 0.2 mM to (2) elicit PR1-CTL bulk cultures. Clinical-scale expansions will be performed before the adoptive cellular therapy trial with PR1-CTL is begun. Quantitative PCR for bcr-abl/abl transcript ratios will be performed monthly to determine whether there is disease progression. If the ratio is increased by ≧2-fold and if the patient has GVHD of <2, then the next higher dose of bulk PR1-CTL will be infused. If there continues to be progression, an unmanipulated DLI from the original donor will be infused at a dose of 1×108 CD3 T cells/kg. However, if there is a decrease in the bcr abl/abl, patients will be monitored monthly until there is no increase for 2 consecutive months. In this way, dose escalations within each patient and amongst cohorts of 3 patients each will be carried out. If patients develop >grade 2 GVHD after the first PR1-CTL infusion, then a selected PR1-CTL infusion will be given in escalating doses starting at 1×10^{5, 5×10}⁵ and 1×10⁶ PR1-CTL/kg of recipient weight. The exact number of PR1-CTL will be determined by tetramer staining, and the PR1-CTL will be selected based on the optimal method determined above. In preliminary experiments, it was shown that peptide/tetramer-coated microbeads, in addition to PR1/HLA-A2 tetramer-based high speed cell sorting using the MoFlo cytometer can be used to enrich for a highly purified PR1-CTL population.

Since patients that receive PR1-CTL might have an initial rise in tetramer+ cells and a simultaneous clinical response, it is possible they could lose immunity and suffer a relapse. This has been shown for a number of adoptive immunotherapy trials using T cells. Therefore, since in the present invention it has been shown that the PR1 peptide vaccine can elicit PR1-CTL in patients with leukemia, it would be prudent to administer the PR1 vaccine to patients after adoptive transfer while there is a minimal residual disease state (<10% Ph+ cells, or PCR+ only) and before the tumor burden becomes too large. A large tumor burden may tip the scale in favor of the leukemia due to PR1-CTL apoptosis induced by the leukemia cells. Therefore, the protocol of the clinical trial will contain a provision for patients to receive the PR1 vaccine at a dose of 0.25 mg SQ every 3 weeks for 3 injections, the dose and schedule that has elicited long-lasting PR1-CTL after vaccination. Statistical considerations, including sample size determination and the proper sequencing of within and amongst patient dose escalations will be performed once the clinical scale up is confirmed since PR1-CTL cell dose depends upon the efficiency of the scale up methods.

It is also indicated that high avidity PR1-CTL will preferentially cause a GVL response and contribute to molecular remission without increasing GVHD. Therefore, a randomized control group of patients will receive unmanipulated DLI following a standard treatment approach. All patients will have blood collected at weekly intervals up to 4 weeks beyond the last infusion of PR1-CTL or DLI, then monthly thereafter until one year or removal from study. Bone marrow biopsies will be performed on day 30, day 60, then monthly until 6 months, then again at 1 year or until removal from study. The overall percentage of functional PR1-CTL in the recipients will be determined by tetramer staining and standard CFC assays. In addition, the number of pp65-CTL in CMV immune patients will provide an internal control to determine whether the number of PR1-CTL is increased after infusion and whether those cells are long-lived. Likewise, the restorative immunity of unmanipulated DLI will be compared with that of patients receiving adoptively transferred PR1-CTL by determining TREC numbers, CD4 and CD8 counts, and the percentage of CFC+ cells. Based on the findings in the PR1 vaccine trial, the quality of the PR1-CTL response, measured by both TCR avidity and function, is perhaps more important to produce a remission than the more simple measurement of the quantity of PR1-CTL. Therefore, the avidity of the PR1-CTL that exist will be compared in (1) the donor product, (2) the recipient prior to transplant and prior to CTL infusion, and (3) the recipient at time points after infusion using limiting doses of tetramer to stain PBMC. This method will be compared to tetramer dissociation half times to confirm the validity of the measurements. In addition, an aliquot of the PR1-CTL product will be used to study whether the high avidity PR1-CTL preferentially kill the patient's CML by using BM target cells cyropreserved prior to BMT, prior to CTL infusion and BM cells taken at time of molecular remission in a standard cytotoxicity assay. In addition, peptide dose-response CFC assays will be performed, as shown in FIG. 11C for the patients that do not have sufficient cells that can be used as targets in a standard cytotoxicity assay. Although it has been shown that the adoptive transfer of antigen-specific CTL clones does not result in long-lived immunity (Riddell et al., 1992), vaccination with the PR1 peptide does result in long-lived PR1-CTL for up to two years. This apparently contradictory result may be because the PR1-specific response is not confined to a single clone. Thus, the adoptive transfer protocol of the clinical trial will involve a broader oligo-clonal PR1-CTL repertoire expanded ex vivo.

Based on the preliminary data, it is suggested that high avidity PR1-CTL will contribute to complete remission after adoptive transfer without increasing GVHD. Thus, these experiments will allow for the direct comparison of patients who do and those who do not achieve CR, to those who achieve CR but who experience worsening GVHD. In the later patients, PR1/HLA-A2 tetramer staining of PBMC and BM will be compared to cells from GVHD site tissue biopsies to determine whether there are more PR1-CTL at the sites of active GVHD. It is indicated that there would be fewer or no PR1-CTL at these GVHD sites since there is no P3 expression in peripheral tissues such as skin. However, if PR1-CTL are present, this will also be compared to the TCR avidity with those of circulating PR1-CTL. A recent murine model has shown that while GVHD effector cells may be allo-antigen-independent, host APC are instrumental and may be required to initiate GVHD (Teshima et al., 2002; Shlomchik et al., 1999). Although PR1 is a self-antigen, it is suggested that PR1-CTL at sites of GVHD would have lower avidity TCR than circulating PR1-CTL, which would potentially allow more cross-reactivity with other peptide ligands and less specificity for the PR1 ligand. Such an observation would give the appearance that the CTL have little peptide specificity and would be consistent with the observations in the murine systems.

In humans, the data is less clear. Dickinson, et al. (2002) have shown that although minor histocompatibility antigen-specific CTL are present at sites of active GVHD in humans (with specificity for both hematopoietic-restricted HA-2 and the ubiquitously expressed H-Y antigens), only CTL with specificity for the H-Y antigen produce cytokines at GVHD sites. Dickinson, et al. (2002) concluded that ubiquitously expressed minor antigens were the targets of GVHD, although they did not measure or compare TCR avidity in the HA-2-specific CTL. Therefore, the frequency and avidity of HA-Y-specific CTL in the skin and blood of recipients of sex-mismatched transplants will be determined and compared to the PR1-CTL. It will be determined whether deletional tolerance of the high avidity PR1-CTL occurs after adoptive transfer and compare this to clinical outcome. It is expected that the selective loss of high avidity PR1-CTL will result from the persistence of CML cells with high P3 expression relative to the P3 expression in BM cells from patients that retain high avidity PR1-CTL and who may be in clinical remission. Deletional tolerance will be determined by comparing the relative disappearance of relatively high and low avidity PR1-CTL after transfer and comparing any disproportionate disappearance of the high avidity PR1-CTL to the rate of disappearance of high avidity pp65-CTL in CMV-immune patients. As shown, CML escape from PR1-CTL immunity may be due to additional mechanisms such as decreased expression of the P3 antigen. Therefore, the amount of P3 expression in the recipient BM and PBMC will be determined by real-time PCR™ and by flow cytometry and compared to the healthy donors. Data regarding general immune status in each of the patients, will serve as a baseline for comparing the specific high avidity PR1-CTL within and amongst patients. In addition, the phenotype data will allow for the comparison of the differentiation state of the PR1-CTL, since an altered maturation phenotype to a terminally differentiated state would be expected to result in ineffective anti-CML immunity.

To determine whether PR1-CTL identified by tetramer staining post-infusion are derived from the adoptively transferred product, TCR-Vβ spectratyping followed by repetitive sequencing of restricted TCR-Vβ families will be performed in selected patients. In the preliminary data it was shown that this technique can be used to determine the breadth of the PR1-CTL response, and was previously used to follow clonal T cell progression in MDS patients (Kochenderfer et al., 2002). The families and the sequences can be compared from PR1/HLA-A2 tetramer-sorted CTL from each of the products and each of the time points to follow the outcome of individual clones. This technique has also recently been used to demonstrate clonal persistence of melanoma-specific CTL following adoptive transfer (Dudley et al., 2002). Spectratype analysis of the HSV Tk-transduced lymphocytes will also be performed to determine TCR repertoire of the transduced lymphocytes during ex vivo expansion. Clones within the transduced cells with similar CDR3 region sequences to PR1-CTL clones would suggest overlapping antigen specificity.

Finally, it has recently been found that 24p3 (the human homologue is NGAL) is secreted by CML cells and inhibits diploid cell proliferation. This factor may also contribute to a loss of proliferative capacity of CML-specific T cells. Serum samples from patients will be collected at each of the indicated time points so that quantitative measurements of NGAL can be determined. If NGAL is expressed at high levels, then cell proliferation by MTT will be determined after exposure of PR1-CTL to NGAL in vitro.

To determine whether PR1 peptide vaccine can be used to elicit PR1-CTL immunity in patients with minimal residual disease after autologous bone marrow transplant or after imatinib mesylate (Gleevec) and interferon. The preliminary results have shown no development of Wegener's granulomatosis or other vasculitis thus far, with only grade 2 toxicity seen in one patient. None of the 3 doses tested were eliminated due to toxicity, and the combined data shows evidence for both immune responses and clinical responses. In addition, it has been shown that the potential for leukemia-induced apoptosis and selective deletion of the most potent antileukemia high avidity PR1-CTL. Data from other laboratories has suggested that vaccination after high dose chemotherapy (such as autologous transplant) may be more effective, however, than vaccination without prior chemotherapy since T cell immunity may be “reset” allowing naïve T cells to expand, possibly including high avidity T cells, if exposed to antigen during the proliferative phase of immune reconstitution after transplant (Borrello et al., 2000). Alternatively, a sufficient expansion of low avidity anti-CML T cells may also be effective in eliminating the leukemia, and this expansion would also be enhanced following transplant (Morgan et al., 1998).

It will be determined whether the PR1 peptide vaccine can elicit PR1-CTL in recipients of autologous BMT for refractory CML. The hypothesis underlying this trial is that PR1 peptide vaccination after autologous BMT will allow re-expansion of high avidity PR1-CTL that were previously eliminated by CML cells. In the absence of any potential autoimmune toxicity, it would be expected that the resulting high avidity PR1-CTL contribute to molecular remission. The efficiency of this expansion of T cells can be measured as an increase in the total number of high avidity cells, an average increase in overall avidity of the induced PR1-CTL, or more rapid expansion kinetics of the high avidity PR1-CTL pool compared to vaccination done without prior transplant. Lastly, the breadth of the PR1 -specific TCR repertoire may increase as well after vaccination, which may reflect a more robust and sustainable immune reaction to the peptide and therefore against the leukemia. The post-transplant PR1 vaccine will be given to recipients of autologous transplantation for CML who have progressive disease and are without a suitable allogeneic donor. The clinical trial will also determine whether PR1 vaccination will induce high avidity PR1-CTL and increase leukemia-free survival in HLA-A2+ patients compared to those without the HLA-A2 allele not given the vaccination (a biological randomization). Based on previous studies, it is expected that no impact of the HLA-A2 allele on outcome in chronic myeloid leukemias will be observed (Cortes et al., 1998). Patients with HLA-A2 will be randomized to receive one of three dose levels of PR1 peptide (0.25 mg, 0.50 mg, and 1.0 mg) given as three injections every 3 weeks. The PR1 peptide will be dissolved in water and mixed with a fixed dose of incomplete Freund's adjuvant (Montanide ISA-51). GM-CSF 75 mg will be administered as a second injection into the same site as the vaccine. Current PR1 vaccine trial, treating patients with AML and MDS with the same vaccination schedule, has not enrolled enough patients to determine whether there is a clear dose effect on the potential to expand high avidity PR1-CTL. However, the most striking example that peptide vaccine alone can induce molecular remission is patient UPN15 who received autologous BMT 2 months prior to vaccination for 2nd relapse of AML and who received the lowest dose (0.25 mg) of the PR1 peptide (FIG. 25).

Bone marrow, and peripheral blood will be collected prior to and again 3 weeks after the last of the 3 vaccine injections, and peripheral blood will be collected every 3 weeks while patients are in the trial. Using these samples, the number of PR1-CTL will be quantify before and after vaccination using tetramers, and by LDA in patients without a detectable response by tetramer staining. PR1-CTL TCR avidity will be determined by using limiting dilutions of MHC-I tetramers, and a titer that produces maximal fluorescence intensity separation of a “high” and “low” intensity population, as discussed previously, will be established for each batch of reagent. TCR-Vβ spectratype will be performed on selected patients in whom the number of PR1-CTL has either increased or decreased by 2-fold or greater to learn whether the breadth of the peptide-specific TCR repertoire has changed. Preliminary experiments were performed on post-vaccination CTL from UPN4 (responder) by comparing the spectratype of total CD8+ lymphocytes, high and low avidity PR1-CTL and the tetramer-negative CD8+ lymphocytes by separating the cells using tetramer labeling and high speed flow cytometry with the MoFlo. It was found that merely two dominant clones in the TCR-Vβ3 family are found within the high avidity PR1-CTL, while multiple clones are dominant in the low avidity PR1-CTL in at least 4 different TCR-Vβ families. Currently the CDR3 regions of the clones are been cloned and sequenced (Kochenderfer et al., 2002) to confirm these initial observations by determining the sequence similarity of the isolated clones. These will be compared to pp65-specific CTL that were also isolated from the patient (a CMV-specific HLA-A2-restricted epitope) since the TCR repertoire should not be influenced by the PR1 vaccine. Preliminary experiments on PBMC samples obtained from UPN4 suggest that the induced high avidity PR1-CTL are derived from a very limited number of clones, which might negatively influence the longevity of these cells. However, it was noted that the patient continues to have 0.58% PR1-CTL in the peripheral circulation 20 months after the last vaccination. It will be important, therefore, to compare the CDR3 sequences of the clones over time in both the responders and the non-responders. This will help us to understand whether there is clonal stability in the PR1-CTL compartment over time or whether clones are gradually replaced over time with a subsequent fine-tuning of the resulting TCR avidity, as has been shown in T cell transgenic mouse models using antigen-specific T cells (Savage et al., 1999).

Because it was observed that TCR avidity correlates with immune response, and because lower doses of PR1 elicit higher avidity PR1-CTL in vitro, it is at first reasonable to believe that lower doses of the PR1 peptide vaccine would produce higher avidity PR1-CTL. However, because of the requirement for cellular uptake and presentation of the injected peptide to elicit PR1-CTL response, a clear dose-response effect may not be established given the heterogeneity of the patients, the different types of leukemia and the different disease stages, number of prior treatments, number of circulating lymphocytes, amongst other variables. Instead, the responses and the resulting avidity of the PR1-CTL will be monitored before and after the trial at each peptide dose and the clinical responders and non-responders groups compared, and the effect of peptide dose on those observations analyzed. The empiric dose range of 0.25 mg to 1.0 mg of PR1 may be outside the range of a more appropriate dose that would have a biologically measurable effect on TCR avidity. Since no other tumor vaccine trial has monitored for these effects, however, these effects will be observed. Furthermore, high numbers of low avidity PR1-CTL, if expanded with the vaccine, may be sufficient to eliminate CML (Molldrem et al., 2002a; Morgan et al., 1998; Molldrem et al., 2002b). As noted, most patients in the current vaccine trial had circulating PR1-CTL detected prior to vaccination. The differences in TCR avidity on all patients will be measured. In addition, CFC analysis for γ-IFN secretion after PR1 peptide stimulation of fresh PBMC samples will be compared at each follow up visit. Peptide doses ranging from 0.2 μM to 200 μM will be used to compare the activation threshold of the CTL and this will be compared to TCR avidity measured with tetramer. For patients that are seropositive for CMV, the HLA-A2-restricted pp65 peptide antigen will be used to compare activation threshold and TCR avidity of the respective peptide-specific CTL.

That PR1 peptide elicits high avidity PR1-CTL in recipients of autologous BMT predicts that pp65-specific CTL will not change the overall avidity after PR1 vaccination. If the avidity is noted to increase, however, it may reflect more general changes in the TCR repertoire in those patients that receive transplant. Therefore, the avidity to CTL from patients that have not received transplant will be compared. An alternative is that the PR1 peptide may fail to elicit any PR1-CTL responses due to nonresponsiveness or anergy. This will be evaluated by measuring surface expression of CD80, CD86, CD28, CTLA-4, and HLA-A2 and HLA-DR expression on BM cells and PR1-CTL. In addition, P3 expression in the BM before and after vaccination will be compared. In addition; whether overexpression of P3 in leukemia cells leads to susceptibility to PR1-CTL-mediated lysis of the leukemia cells, and also preferentially induces apoptosis of high avidity PR1-CTL over low avidity PR1-CTL will be tested. The later will lead to outgrowth of the leukemia. Clinical response to the PR1 vaccine will be compared, as will the P3 expression and the resulting change in the TCR avidity of the induced PR1-CTL in all patients on the vaccine trial. In addition to studying the vaccine patients, samples of blood and marrow are collected on all patients that undergo allogeneic BMT and patients that receive DLI. These samples will be used to examine P3 expression in BM cells and the PR1-CTL avidity of PBMC in HLA-A2+ patients with CML that have relapsed after BMT and subsequently received DLI. Pretransplant, remission and relapse samples will be compared. Currently there are 12 such patients with sufficient samples and cell numbers to be examined.

Based on the data, it is predicted that P3 expression would be high (relative to normal BM) pre-BMT, low at time of remission, high again during relapse, but low after successful BMT or immunotherapy in patients that have a preserved high avidity PR1-CTL response after BMT. In patients that have a high avidity PR1-CTL population at time of remission after BMT and who subsequently lose the high avidity cells after BMT, it is predicted that the BM cells would either have a higher average P3 expression over time than the former group of patients, or that the P3 expression is maintained relatively “high” during remission after BMT, which either increases or do not change substantially after subsequent immunotherapy. In these patients, it is further predicted that there will be no clinical response to immunotherapy with concomitant loss of high avidity PR1-CTL.

Other parameters will likely influence the outcome, as shown in patients UPN4 and UPN6 treated with the PR1 vaccine. Therefore, P3 distribution will also be measured in early versus more committed BM progenitors using flow cytometry, surface expression of CD80, CD86, ICAM-1 (CD54), overall MHC and HLA-A2 expression, and the activation thresholds of the PR1-CTL will be examined using the γ-IFN CFC assay and correlate with TCR avidity measured with tetramer staining. BM and PBMC will also be examined to compare any differences in TCR avidity and P3 expression between the two compartments since BM is the site of a larger number of leukemia cells and high avidity PR1-CTL may be absent in the marrow but still be present in PBMC. Although cause and effect are more difficult to establish in patients compared to animal models where parameters can be more easily manipulated to establish that P3 overexpression in leukemia induces selective loss of high avidity leukemia-specific T cells, it is expected that these experiments are likely to yield important insights into the nature of tolerance since the antigen is well-defined, the tools are established and the patient samples necessary to conduct the experiments are avaiable.

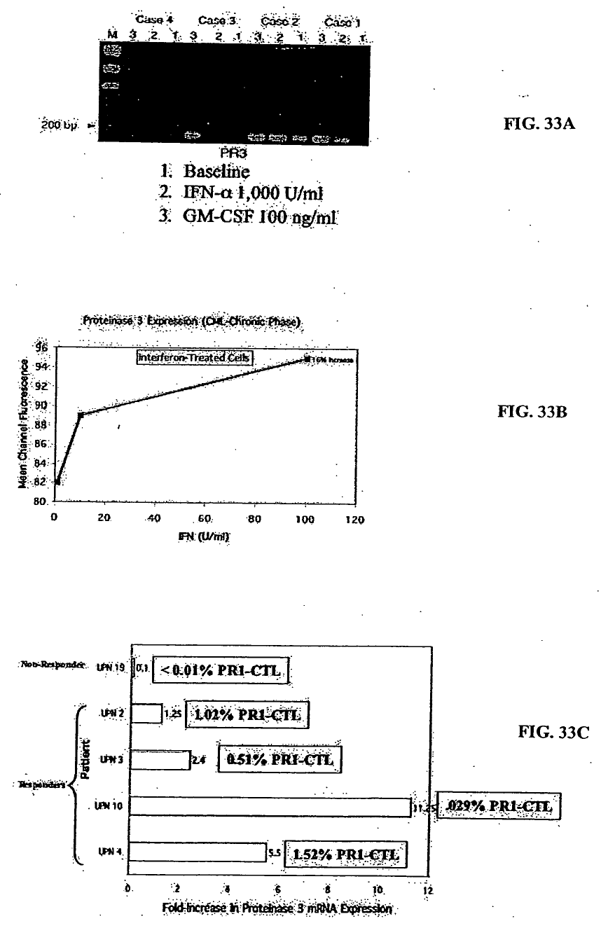

The inventors and others (Burchert et al., 2002) have also found that IFN induces P3 expression in myeloid cells. This increased expression may alter susceptibility to PR1-CTL-mediated killing, but it may also cause apoptosis of the PR1-CTL. In preliminary experiments, it has been shown that IFN preferentially upregulates P3 only in the patients that achieve cytogenetic remission after treatment with IFN, and in whom a high avidity PR1-CTL immune response is noted by tetramer staining (FIG. 33). This may be interpreted to mean that P3 overexpression does not lead to selective loss of high avidity PR1-CTL. However, other effects of IFN on the PR1-CTL may influence the survival of the high avidity cells since type I interferons are known to suppress apoptosis in activated antigen-specific T lymphocytes (Stark et al., 1998; Tanaka et al., 1998; Nakamoto et al., 1997). Therefore, using cryopreserved BM and PBMC from CML patients, will incubate high and low avidity PR1-CTL with CML cells as was done previously, but with and without INF-α and IFN-γ and with or without antibody to IFN-α or IFN-γ. to determine whether either IFN prevents PR1-CTL apoptosis (in addition to increasing P3 expression). If true, this may also help to explain the usefulness of IFN in the treatment of other solid tumors that are also targeted by tumor-specific CTL, such as melanoma and renal cell carcinoma. Lastly, the murine model of IFN-transduced mesenchymal stem cells (MSC) to treat CML may also give clues related to tolerance. For instance, increased expression of P3 after induction of IFN expression may increase susceptibility to immune attack by P3-specific T cells, while at the same time leading to their ultimate extinction by apoptosis.

In the clinical trial whether the PR1 vaccine, administered as 3 injections every 3 weeks as previously described, will induce a PR1-CTL response and whether the vaccine induces cytogenetic and molecular responses in CML patients that are refractory to imatinib mesylate (Gleevec) will be determined. It is expected that the PR1 vaccine will induce PR1-CTL in patients that are refractory to imatinib and improve cytogenetic remissions in those patients. It is also expected that potential downregulation of P3 by treatment with imatinib may facilitate expansion of high avidity PR1-CTL induced by the vaccine. Patients will be randomized on the basis of HLA-A2 expression to receive IFN alone versus PR1 peptide with or without IFN in patients with 10% to 90% Ph+ disease after 9 months of imatinib therapy. P3 in BM samples cryopreserved at the time of diagnosis (when available) will be quantified and compared to the time of study entry, and again 3 weeks following the vaccine. The P3 expression in vaccine recipients will be compared to patients that receive IFN alone or IFN plus PR1. Because imatinib down-regulates P3 while IFN upregulates P3 expression, it is not possible to predict which mechanism might prevail to alter P3 expression. Concomitant to examining BM samples from the clinical study, however, the effect of both agents on P3 expression on BM cells from healthy donors and newly diagnosed CML patients in vitro will be examined. Epigenetic changes, such as methylation of the P3 gene, lead to altered expression of P3 (Lubbert et al., 1999), which may be independent of the effects of either ifnatinib or IFN and this will be investigated in patient samples. In addition, because it was found that high avidity PR1-CTL undergo apoptosis when exposed to high doses of PR1, it is expected that very low expression of P3 in imatinib-treated patients may facilitate the reemergence of previously depleted high avidity PR1-CTL. Whether the high avidity PR1-CTL are present prior to vaccination, and the relative influence of the vaccine, compared to IFN on their possible reemergence will be determined. CFC studies will be performed as previously described, to determine whether there are differences in functional activity in either the low or the high avidity PR1-CTL. Phenotypic data will be used to compare the maturation state of the CTL, which may relate to generalized T cell non-responsiveness.

### Example 11

### Myeloperoxidase (MPO)

More recently, the inventors have studied another myeloid-restricted protein, Myeloperoxidase (MPO), a heme protein synthesized during early myeloid differentiation that constitutes the major component of neutrophil azurophilic granules.

It was found that MY4 (RLFEQVMRI (SEQ ID NO:30)), a 9 aa peptide derived from MPO that binds to HLA-A2.1, can be used to elicit CTL from HLA-A2.1+ normal donors in vitro (Braunschweig et al., 2000). These MY4-specific CTL show preferential cytotoxicity toward allogeneic HLA-A2.1+ myeloid leukemia cells over HLA-identical normal donor marrow (Braunschweig et al., 2000). MY4-specific CTL also inhibit colony forming unit granulocyte-macrophage (CFU-GM) from the marrow of CML patients, but not CFU-GM from normal HLA-matched donors. Like PR1, MY4 is therefore a peptide antigen that can elicit specific CTL lysis of fresh human myeloid leukemia cells. Other peptides from MPO are predicted to bind to HLA-A2.1, but not all of these have been tested for their potential to stimulate immunity.

Some previous studies have established PR1 to be an important leukemia-associated antigen (LAA), and because of the many striking similarities of the nature of the immunity directed against Pr3 and MPO, it is likely that similar methods applied to the study of MPO-specific immunity will establish MY4 and potentially other MPO peptides as important LAA as well (Kochenderfer and Molldrem, 2001).

### Example 12

### Peptide Selection and Binding Assays

In the first step to generating T cells which could be used for adoptive immunotherapy of myeloid leukemias, several peptides derived from the published sequence of MPO have been identified which were predicted to bind to HLA-A*0201 using a published algorithm (Molldrem et al., 1996; Parker et al., 1994). This allele was chosen because its high frequency in the US population (49% of individuals) would maximize the therapeutic relevance of any eventual immunotherapeutic strategy. Of 10 peptides predicted to have sufficiently high binding affinities based on the known HLAA2.1 binding motif, the five with the highest predicted binding were subsequently synthesized (designated MY1 through MY5) (Table 14). The peptides were synthesized by Biosynthesis (Lewisville, Tex.) or by the M. D. Anderson Protein CORE Facility (Houston, Tex.) to a minimum of 95% purity as measured by high-performance liquid chromatography (HPLC). Peptide binding to HLA-A2.1 was confirmed using two assays. In the first, indirect flow cytometry was used to measure HLA-A2.1 surface expression on the A2+T2 cell line coated with the peptide. T2 cells are a human lymphocyte line that lacks TAP1 and 2 genes and cannot therefore present endogenous MHC class I restricted antigens. If the peptide effectively bound HLA-A2.1, it stabilized the complex with β2-microglobulin and increased HLA-A2.1 surface expression, which could be measured using flow cytometry. An HLA-A2.1 specific monoclonal antibody (BB7.2, ATCC, Rockville, Md.) followed by a FITC-labeled secondary antibody (CALTAG) was used to measure surface expression of HLA-A2.1. In the second assay, the dissociation rate of I¹²⁵-labeled β2-microglobulin from the heterotrimer complex of the HLA-A2.1 heavy chain, peptide, and β2-microglobulin was measured, which allowed calculation of binding half-life (t_{1/2}). The labeled heterotrimer complex was separated from unincorporated β2-microglobulin by high-performance liquid

TABLE 14

Peptide Start Amino Acid (aa) Subsequence
Residue Half-Time Position Disassociation

| | | | | |
|---|---|---|---|---|
| | | | SEQ | Residue Half-Time |
| | Start | Subsequence | ID | Position |
| Peptide | (aa) | Residue | NO: | Disassociation |

| | | | | |
|---|---|---|---|---|
| MY1 | 132 | SLWRRPFNV | 31 | 3348.233 |
| MY2 | 28 | KLLLALAGL | 32 | 636.279 |
| MY3 | 98 | LLSYFKQPV | 33 | 449.306 |
| MY4 | 571 | RLFEQVMRI | 30 | 364.011 |
| MY5 | 504 | LIQPFMFRL | 34 | 253.129 |
| MY6 | 529 | RVFFASWRV | 35 | 168.881 |
| MY7 | 53 | VLGEVDTSL | 36 | 148.896 |
| MY8 | 418 | LLLREHNRL | 37 | 134.369 |
| MY9 | 143 | VLTPAQLNV | 38 | 118.238 |
| MY10 | 118 | YLHVALDLL | 39 | 110.747 |

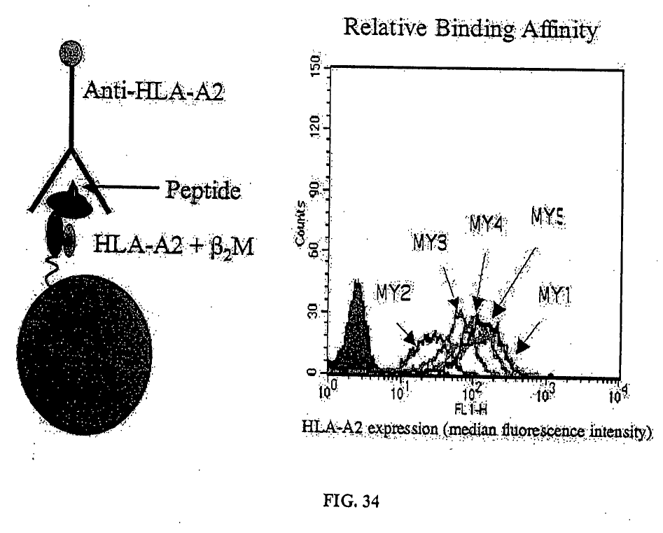

MPO peptides predicted to bind HLA-A2.1 chromatography gel filtration, and the halftime of disassociation of β2-microglobulin were determined by subjecting aliquots of the complex to a second round of gel filtration. Five peptides showed increased surface HLA-A2.1 expression compared with T2 cells with no added peptide (background HLA-A2.1 expression; (FIG. 34; Braunschweig et al., 2000). The control peptides are the PR1 peptide and an Influenza B nucleoprotein (aa 85-94; Flu), both with known high binding affinity to HLA-A2.1. The long measured t_{1/2} as measured using β2-microglobulin disassociation confirmed the binding of MY1 through MY5 to HLAA2.1 (Molldrem et al., 1996).

### Example 13

### Induction of Primary CTL Responses to Peptides

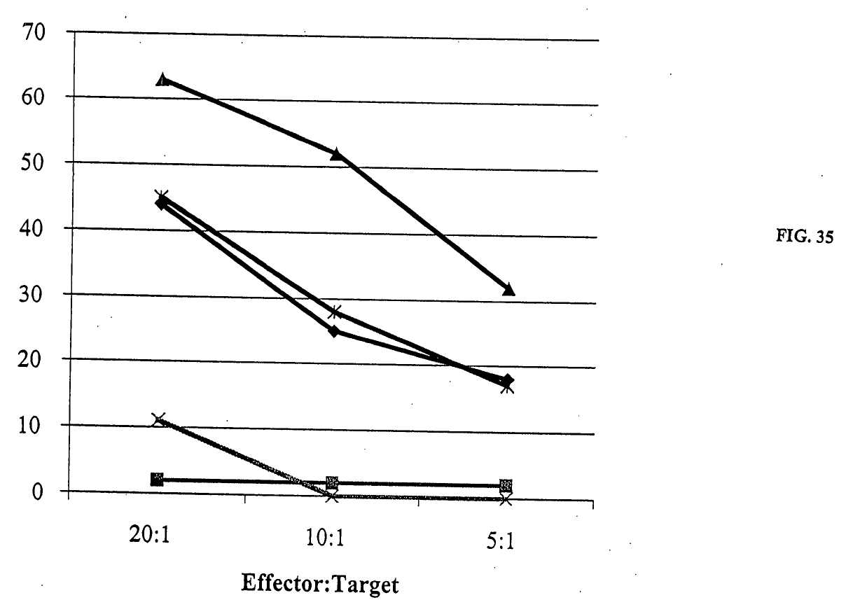

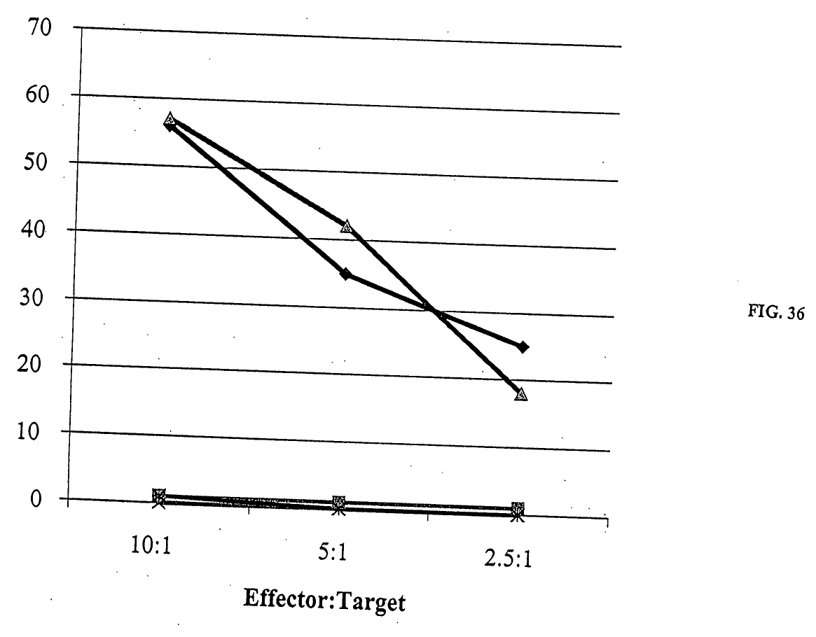

The five MPO peptides discussed above, were used to stimulate T cells specific for peptide-coated targets. PBMC from a normal healthy donor heterozygous for HLA-A2.1 were stimulated with peptide-pulsed T2 cells. The T2 cell line has been used by others as an antigen presenting cell for the generation of peptide-specific CTL. Briefly, T2 cells (which co-express the costimulatory molecule B7.1) were washed 3 times in serum-free RPMI culture media supplemented with penicillin/streptomycin and glutamine (CM) and incubated with peptide at concentrations ranging from 0.2 to 200 μg/mL for 2 hr in CM. The peptide loaded T2 cells were then irradiated with 7500 cGy, washed once, and suspended with freshly isolated PBMC at a 1:1 ratio in CM supplemented with 10% human serum (HS) (Sigma, St. Louis, Mo.). After 7 days in culture, a second stimulation was performed and the following day, 60 IU/mL of recombinant human interleukin-2 (rhIL-2) (Biosource International, Camarillo, Calif.) was added. After 14 days in culture a third stimulation was performed, followed on day 15 by addition of rhIL-2. A fourth stimulation was performed on day 21 followed on day 22 by the addition of rhIL-2. After a total of 27 days in culture, the peptide-stimulated T cells were harvested and tested for peptide specific cytotoxicity toward CalceinAM labeled T2 cells, leukemia cell lines, and fresh human leukemia cells. FIGS. 35 and 36 show the peptide specific lysis of the CTL lines against T2 cells loaded with either 2.0 μg/mL of MY2 or MY4, or T2 cells without added peptide, at varying effector to target (E:T) ratios. No peptide specific CTL lines could be elicited using MY1, MY3 or MY5 peptides, despite testing using different donors and differing peptide concentrations. The CTL line generated against the MY2 peptide demonstrated high specific lysis against MY2-loaded target cells, whereas the CTL line generated against MY4 did not demonstrate any significant cytotoxicity against MY2-loaded targets (Molldrem et al., 1996). The converse experiment, using CTL generated against MY4, tested similarly. Cytotoxicity toward T2 cells loaded with HTLV-1 tax (aa 11-19), an irrelevant peptide with high binding affinity to HLAA2.1, was also measured and resulted in <20% specific lysis at E:T ratios of 50:1 by CTL specific for either MY2 or MY4. CTL stimulated weekly with either higher or lower peptide concentrations of MY2 or MY4 did not produce a short-term CTL line by 4 to 6 wk, as measured by specific lysis of peptide-coated T2 targets at the end of culture. Only CTL stimulated with 2.0 μg/ml of peptide produced effective short-term CTL lines. This observation was reproducible across 10 healthy HLA-A2.1+donor PBMCs that were used in the studies to elicit the CTL. This phenomenon was noted previously for CTL elicited against other self-peptides.

### Example 14

### HLA-A2.1 Restricted CTL Responses

To further demonstrate that the CTL response toward MY2 or MY4 are specific for target cells expressing the HLA-A2.1 molecule, T2 cells loaded or not loaded with 2.0 pg/mL MY2 or MY4 were prepared. The CTL line generated against the respective peptides were also used to test for specific lysis. Mouse monoclonal antibody against HLA-A2.1 (BB7.2) was used to block HLA-A2.1-restricted recognition by the CTL line. T2 cells without peptide, but with antibody present, were used to control for any potential non-specific antibody-mediated cytotoxicity.

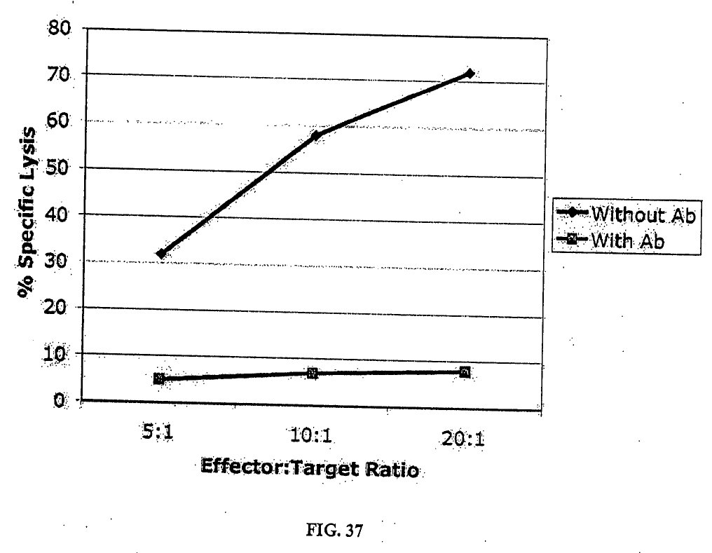

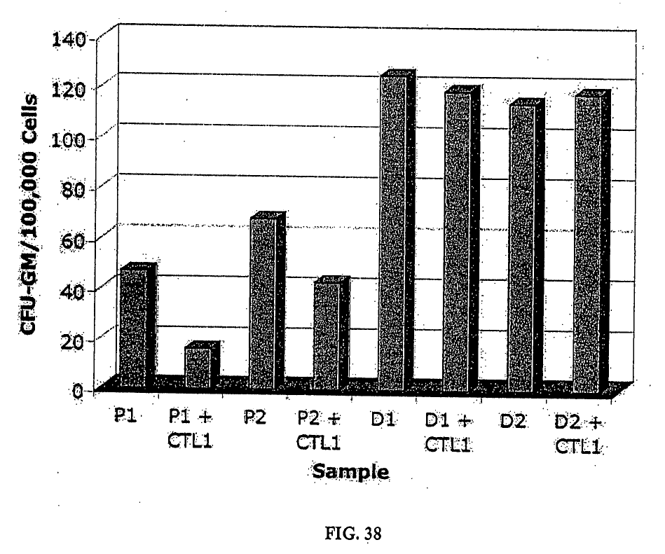

FIG. 37 demonstrates that with the addition of antibody to HLA-A2.1, specific lysis was blocked. Further, there was only background lysis of T2 cells in the presence of antibody alone. The data shown in FIG. 37 is the combined results from three separate experiments using three separately generated CTL lines. This demonstrated that the observed cytotoxicity was HLA-A2.1-restricted.

### Example 15

### PR1 Specific CTL Preferentially Lyse Human Myeloid Leukemia Cells

It was next determined whether the MY2 and MY4-specific CTL lines were capable of lysing allogeneic human myeloid leukemia cells from HLA-A2.1 positive individuals. The targets were BM cells from pre-transplant HLA-A2.1-positive AML patients. As controls, BM cells from HLA-A2.1-negative AML patients, BM from HLA-A2.1-positive healthy donors and two cell lines expressing low levels of MPO were used: HLA-A2.1 transfected K562 cells and the U937 cell line which lacks HLA-A2.1 and would therefore be incapable of presenting peptides in an HLA-A2.1 -restricted manner.

FIG. 35 shows the combined results of three separate experiments from three MY4-specific CTL lines. The specific lysis by MY4-specific CTL, at various E:T ratios, of either BM from healthy HLA-A2.1-positive donors, HLA-A2.1-positive AML patients, HLA-A2.1-negative AML patients, or T2 cells with or without exogenously added MY4 peptide at 2.0 μg/mL is shown. The specific lysis of U937 and HLA-A2.1-positive K562 cells by MY4-specific CTL was lower than the background lysis observed against T2 cells without added peptide. The results show that CTL elicited with the MY4 peptide result in short-term CTL lines with both MY4 and HLA-A2.1 specificity that killed AML cells, but not normal cells.

In contrast, FIG. 36 demonstrates typical cytotoxicity results from three experiments with three different MY2-specific CTL lines. Marrow cells from patients with HLA-A2.1-positive AML were readily lysed, at an E:T ratio of only 5:1. However, marrow cells taken from an HLA-A2.1-positive normal healthy donor also demonstrated significant lysis (43% lysis at E:T of 20:1), similar to that of the HLA-A2.1-positive AML cells.

### Example 16

### MY2- and MY4-Specific CTL Lysis of Leukemia Cells is Associated with Abberant MPO Expression

All target cells were assayed for the presence of cytoplasmic MPO. After permeabilizing the cell membrane with Ortho PermeaFix (Ortho Diagnostics, Raritan, N.J.), staining was performed using a FITC-labeled antibody to MPO (Accurate Chemicals, Westbury, N.Y.) and a PE-labeled antibody to CD34 (Becton-Dickinson, San Jose, Calif.) followed by flow cytometry.

Table 15 lists the percentage of cells in the sample population that stain positive for MPO, as well as the median fluorescence intensity of intracellular MPO staining. The percentage of cells expressing surface MHC class I and CD80 (the costimulatory molecule B7.1) was also evaluated in the same target cell populations by staining with FITC labeled antibodies. These experiments demonstrate that it is possible to elicit CTL specific for the MY2 and MY4 self-peptides from normal HLA-A2.1-positive donors that exhibit in vitro cytotoxicity against myeloid leukemia cells. Furthermore, the degree of cytotoxicity was associated with aberrant MPO expression. However, the MY2-specific CTL also showed specific lysis of normal donor marrow cells, which suggests that immunity elicited against this peptide in vivo might result in autoimmunity that would be incapable of distinguishing leukemic cells from normal marrow progenitor cells. Lastly, since the CTL were tested against whole marrow from leukemia patients in short-term assays, it was possible that leukemia progenitor cells, which might not aberrantly express MPO, could escape CTL recognition. Therefore, whether leukemia progenitor cells could be eliminated by MY4-specific CTL in an AML colony-forming assay was investigated. The MPO expression in both leukemia and normal CD34+ cells was also determined.

TABLE 15
Cytoplasmic MPO Expression and Surface Phenotype of Target Cells
Used in Cytotoxicity Experiments

| | | % Cells | | % Cells | % Cells |
|---|---|---|---|---|---|
| | | Expressing | MFI of MPO | Expressing | Expressing |
| Patient/Donor | Target Cell* | MPO | Expression | MHC I | CD 80 |
| AML #1 | For MY2-CTL | 98 | 982 | 88 | 46 |
| AML #2 | For MY2-CTL | 77 | 841 | 92 | 38 |
| AML #3 | For MY4-CTL | 89 | 966 | 98 | 58 |
| AML #4 | For MY4-CTL | 84 | 859 | 83 | 41 |
| Donor #1 | For MY2-CTL | 14 | 254 | 99 | 80 |
| Donor #2 | For MY4-CTL | 18 | 217 | 96 | 56 |
| U937 | For both CTL | 90 | 212 | 100 | 23 |
| K562 | For both CTL | 21 | 181 | 28 | 32 |

*Target for specified cytotoxicity experiment;
MPO = myeloperoxidase;
MFI = median fluorescence intensity.

### Example 17

### MY4-Specific CTL Preferentially Inhibit AML Colony-Forming Units

PBMC from two normal healthy donors heterozygous for HLA-A2.1 were stimulated with peptide-pulsed T2 cells using the method previously described. FIG. 38 shows the results of colony inhibition assays using CTL derived from a 13 day MY4 peptide-pulsed culture (CTL1). CFU-GM from patient P1 (M2-AML) and P2 (M4-AML) showed 63% (p =0.006) and 34% (p =0.007) inhibition, respectively. In contrast there was no inhibition of CFUGM from normal marrow, D1 and D2, the corresponding HLA identical marrow donors for P1 and P2. Control CTL1 plated alone in methylcellulose under identical experimental conditions at 5×10⁵ cells/ml showed no CFU-GM by day 16.

### Example 18

### MPO is Expressed in Leukemic CD34+ Cells—Expression is Limited to Hemapoietic Cells

Next it was confirmed that MPO was expressed in early CD34 positive CML cells. Marrow was obtained from a patient with CML in CP, a patient with AML, and normal CD34 cells from G-CSF mobilized peripheral blood mononuclear cells from a normal donor for comparison. Cells were first labeled with PE conjugated anti-CD34 antibody (Becton Dickinson, San Jose, Calif.), followed by cytoplasmic staining for MPO.

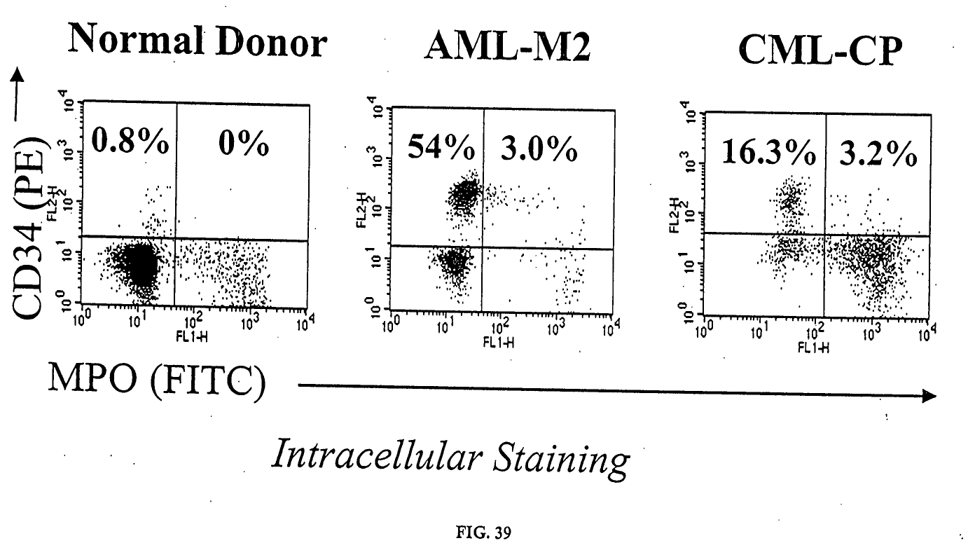

FIG. 39 shows that 19% of the CML cells were CD34 positive and 16% of those cells highly expressed Pr3. In addition, CD34-negative cells also expressed MPO. In contrast, none of the normal CD34 positive cells expressed MPO. In cells from another patient with AML, 57% of AML cells were CD34-positive and 5% of those cells highly expressed MPO. This shows that very early progenitor cells overexpress MPO whereas there is no MPO expression normal progenitor cells.

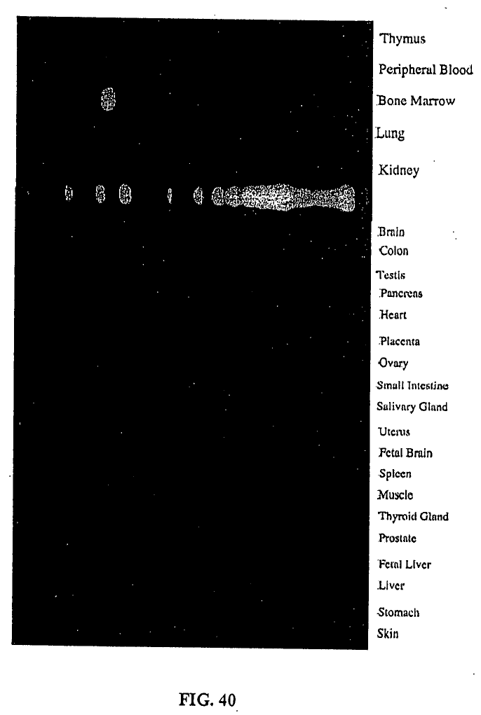

To confirm that MPO expression was limited to hematopoietic cells, a panel of human tissues for MPO RNA expression was analyzed using RT-PCR. Following isolation and reverse transcription of RNA from each of the tissue samples, cDNA was amplified with 30 cycles of PCR. Primers amplifying a region spanning the 3rd and 4^{th} coding regions (conjunction position 613, 5′ primer 588-610 (CATCTGCTTCGGAGACTCAGGTG (SEQ ID NO:40)), 3′ primer 689-672 (TCAGGGAAAAGGCGGGTG (SEQ ID NO:41)) were selected and used to generate PCR products that were then separated on a 2% agarose gel. The gel was imaged on a BioRad analyzer and GelDoc software was used to quantify the products. FIG. 40 shows that expression of MPO is limited to bone marrow.

### Example 19

### MY4-Specific CTL Identified in Peripheral Blood of Recipients of Nonmyeloablative Stem Cell Transplants using Peptide-HLA-A2 Tetramers

Because CTL lines against both MY2 and MY4 could be elicited from normal donors and that killed AML, it was next assessed whether it was possible to detect these CTL in the peripheral blood of patients with AML. In contrast to CTL with specificity for MY4, MY2-specific CTL caused lysis of both leukemic and healthy bone marrow cells, which suggested that it would be unlikely to find high circulating numbers of MY2-specific CTL since these might mediate autoimmunity in addition to anti-leukemia immunity. Previous successful methodology using peptide/HLAA2 tetramers to identify the similarly deduced peptide PR1 as an HLA-A2.1-restricted leukemia associated antigen (LAA) (Molldrem et al., 2000) argued strongly that this same methodology could be used to determine whether MY2 and MY4 were also potential LAAs.

Production of peptide/MHC tetramers has been described by in detail elsewhere (Molldrem et al., 2000). Briefly, a 15 amino acid substrate peptide (BSP) for BirA dependent biotinylation has been engineered onto the COOH terminus of HLA A2. The A2 BSP fusion protein and human β2 microglobulin (β₂M) were expressed in E. coli, and were folded in vitro with the specific peptide ligand. The properly folded MHC peptide complexes were extensively purified using FPLC and anion exchange, and biotinylated on a single lysine within the BSP using the BirA enzyme (Avidity, Denver, Colo.). Tetramers were produced by mixing the biotinylated MHC peptide complexes with phycoerythrin (PE) conjugated Neutravidin (Molecular Probes), or PE(Cy7)-conjugated Neutravidin at a molar ratio of 4:1. MY2 and MY4 tetramers were validated by staining against a CTL line specific for each peptide. CMV tetramers were validated by staining with PBMC from a CMV immune individual. Specificity was demonstrated by the lack of staining of irrelevant CTL. By titrating positive CTLs into PBMCs from normal controls, the limit of detection was established to be as low as 0.01% of CD8+ cells. Each tetramer reagent was titered individually and used at the optimum concentration, generally 20 μg/ml-50 μg/ml.

Nine HLA-A2.1-positive AML patients in relapse prior to allogeneic NST, and then again at day 60 post-transplant were examined using peptide/MHC tetramers. All of the patients were CMV immune prior to transplant, and therefore pp65/HLA-A2.1 tetramers served as positive controls for evidence of antigen specific immunity. Because of the limited amount of sample that was available, staining method was modified to use 2 or more different tetramers simultaneously to stain PBMC samples.

In contrast to MY2-CTL, which showed 45% specific lysis of HLA-A2+ normal BMC in addition to killing leukemia, MY4-CTL showed no lysis of normal BMC and only killed leukemia cells, which suggested that MY4 may be a more biologically relevant leukemia antigen. It was predicted that high numbers of circulating MY2-CTL would not be found, but that MY4-CTL may be detectable in leukemia patients. CTL from blood samples obtained 60 days after NST in 9 HLA-A2 μML patients were studied using combinations of 6 HLA-A2 tetramers and multiparameter flow cytometry using a MoFlo cytometer (Cytomation, Fort Collins, Colo.). A2 tetramers were constructed with the following peptides: PR1, MY2, MY4, the CMV pp65 peptide, and the minor antigens HIA-1R (a negative control) (den Haan et al., 1998) and HA-1H (the allele against which CTL responses have been shown) (den Haan et al., 1995; den Haan et al., 1998; Marijt et al., 1995). All patients showed evidence of donor chimerism by DNA microsatellite analysis at the time of study, and all were CMV seropositive.

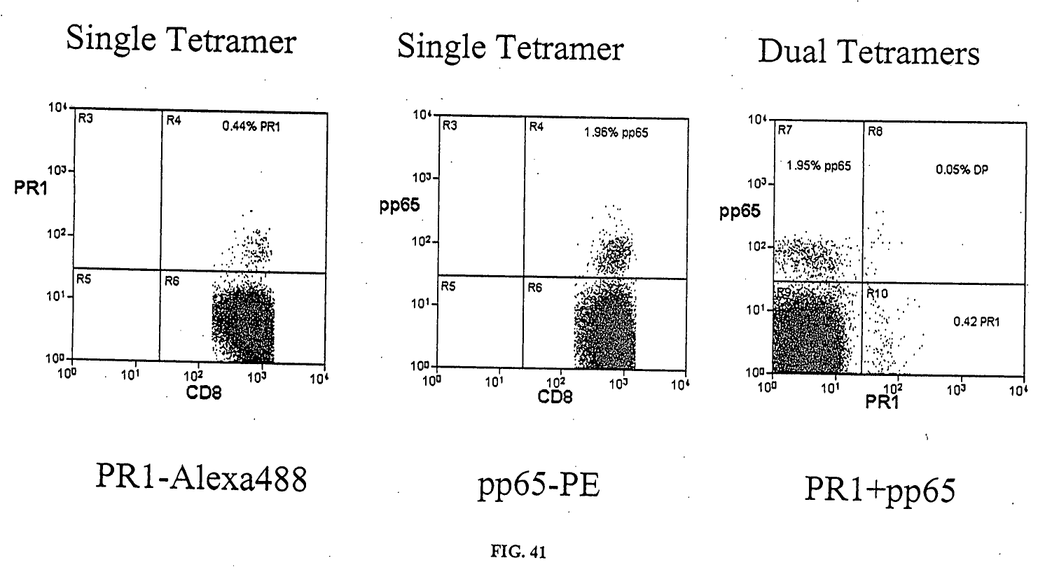

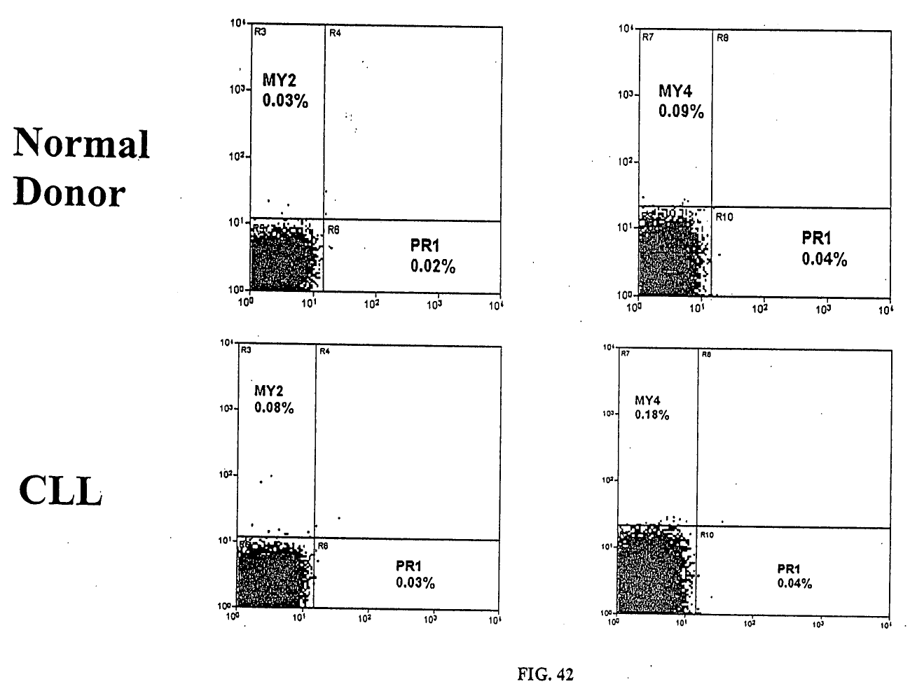

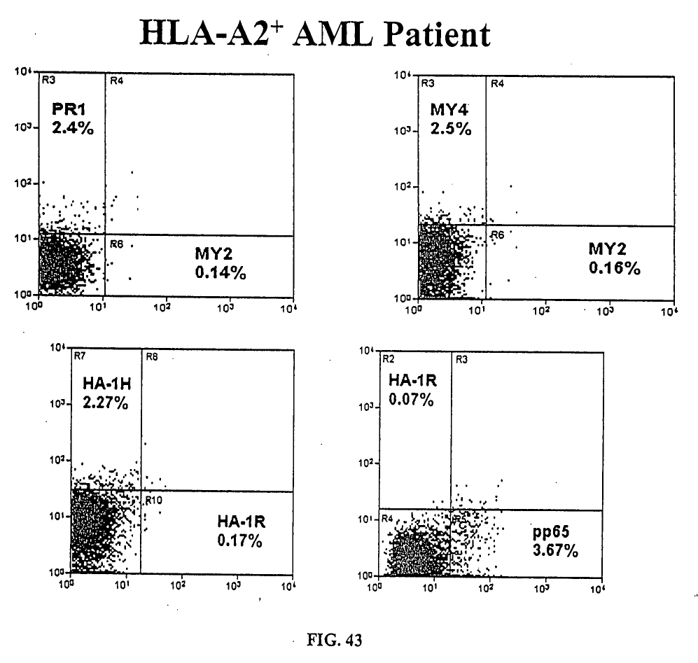

FIG. 41 shows that multiple-tetramer staining is associated with a 0.1% loss of sensitivity; however, ratios of antigen specific CTL were preserved when compared to single tetramer staining. As shown in FIG. 42, staining PBMC from HLA-A2.1-positive healthy donors and from patients with lymphoid-derived tumors (multiple myeloma and chronic lymphocytic leukemia) showed there was no detectable CTL immunity against myeloid-specific antigens. However, as shown in FIG. 43, both PR1-CTL and MY4-CTL are detected in the peripheral blood of these patients, but MY2-CTL is not detected (the limit of sensitivity is 0.01% in cell titration experiments). This figure shows a representative patient sample of peripheral blood that was obtained on day 60 post-NST and stained using the multiple tetramer methodology and CD8 and then analyzed using CellQuest software.

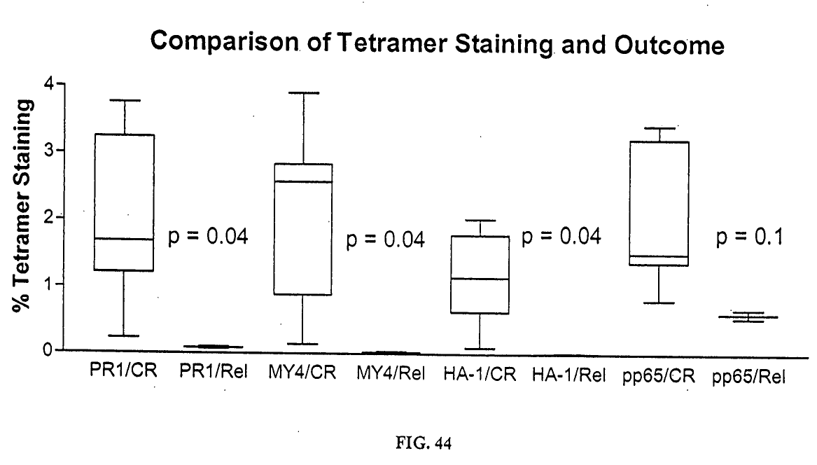

Next, a total of 9 HLA-A2+ NST recipients were examined on day 60 post-transplant for immunity against each of the 6 peptides. By comparing relapse rates, as summarized in FIG. 44, a greater percentage of PR1-CTL were present in patients in continuous remission at 1 year (median of 2.45%) versus those patients that went on to relapse (median of 1.55%), p=0.03. Although there was a trend toward a higher percentage of both MY4- and HA-1H-CTL in patients in remission, it did not reach statistical significance. No MY2-CTL were identified in any of the patients studied. The development of either ≧grade II acute GvHD or chronic GvHD did not correlate with the percentage of any of the peptide-specific CTL studied, although there was a trend toward a higher number of HA-1-CTL in patients that developed significant GvHD. Importantly, these preliminary experiments (1) extended the importance of PR1-specific CTL immunity to NST recipients, (2) highlighted the probable relevance of MY4-specific CTL immunity in NST recipients, and (3) lessened the likelihood of biological relevance of MY2-specific CTL immunity as part of the anti-leukemia response.

This is the first study to combine simultaneous multi-tetramer analysis with other surface phenotypic markers to determine the relative importance of leukemia-associated antigens in the GVL effect. These results indicate that although CTL with MY2 specificity are below the detection limit using tetramer staining, MY4-CTL and HA-1H-CTL are increased in NST recipients and may therefore be important in the GVL effect after NST for AML. These results also extend the previous results and suggest that PR1-CTL may also contribute to the elimination of AML after NST.

### Example 20

### Sorted Antigen-Specific CTL Specifically Kill Leukemia Cells

To show that tetramer-sorted antigen-specific CTL are functional, PR1/HLA-A2 tetraner+ CTL from a donor lymphocyte (DLI) product obtained from leukapheresis were stained, sorted and tested for lysis of both donor and recipient (which contained >90% blasts) cryopreserved BM. The recipient was in remission by 6 months after allogeneic BMT, but relapsed with chronic phase CML by 12 months with 100% Ph+ BM cells. The patient was then treated with a total of 7×10⁷ DLI per kilogram body weight from months 12 to 13 with no other therapy, and was in remission with 0% Ph+ BM cells by month 18 when PBMC were available for testing.

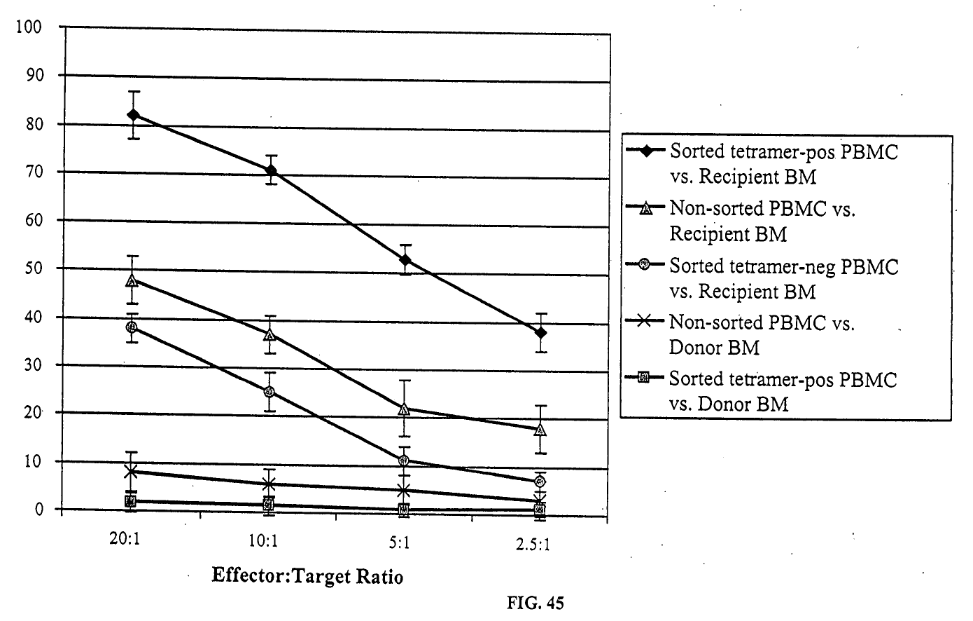

After staining with the PR1/HLA-A2 tetramer and sorting on the MoFlo cytometer, the yield of sorted PR1/HLA-A2 tetramer+cells was 81%, with 90% purity, and the sorted tetramer-negative population contained no detectable PR1/HLA-A2 tetramer+ CTL. As shown in FIG. 45, the sorted PR1/HLA-A2 tetramer positive CTL showed greater lysis of recipient marrow taken from time of relapse than the non-sorted PBMC. Although the sorted PR1/HLA-A2 tetramer negative CTL showed less lysis of recipient BM than non-sorted PBMC, it was above background lysis against donor BM. This likely reflects non-PR1-specific CTL with activity against other leukemia antigens. Minimal or no lysis of donor BM was seen with either the sorted PR1/HLA-A2 tetramer positive CTL or non-sorted PBMC. This is strong evidence that PR1-specific CTL actively lyse CML cells and contributed to remission in this patient (Molldrem et al., 2000).

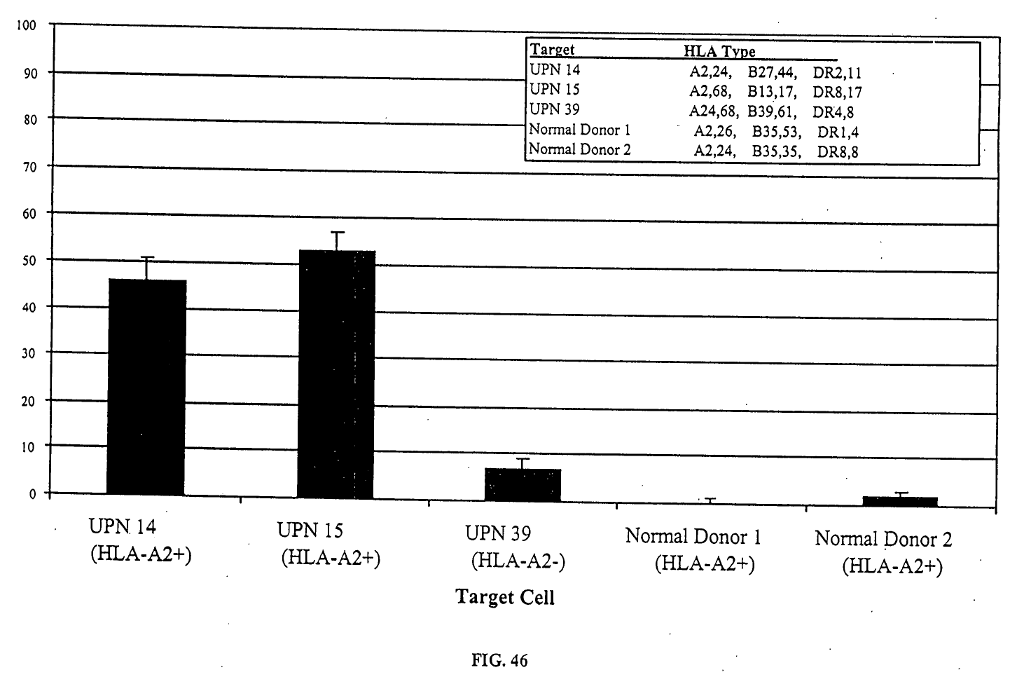

To confirm leukemia specificity of the PR1-specific CTL, PBMC from the recipient were again sorted using the PR1/HLA-A2 tetramer and tested for lysis of HLA mismatched target cells. As shown in FIG. 46, PR1/HLA-A2 tetramer sorted PBMC showed lysis of HLA-A2.1+ CML cells from 2 unrelated patients, but no lysis of either HLA-A2.1-CML cells or HLA-A2.1+ normal donor marrow cells at E:T ratio of 5:1.

To show that CTL with specificity for peptides such as MY4, which are identified using a deductive strategy, can successfully be translated to the clinic, a phase I clinical trial using the PR1 peptide as a vaccine with incomplete Freund's adjuvant and GM-CSF was initiated. This further demonstrates that highly useful LAA can be identified using these methods.

Patients eligible for the vaccine included HLA-A2+ CML and AML patients that had failed conventional therapy or AML patients that were in 2nd CR (i.e. at high risk of relapse). When PR1 was administered subcutaneously at 0.25, 0.5 or 1.0 mg every 3 weeks for 3 injections, PR1-specific CTL immunity was elicited in 6 of 9 patients (by tetramer staining) and complete remissions were obtained in 2 patients (1 μML and 1 CML patient). The patient with AML was positive for the t(15;17) translocation and subsequently became PCR negative after vaccination. Importantly, the expanded PR1-specific CTL from peripheral blood of that patient were isolated by tetramer staining and relapsed BM cells were killed, but not BM cells taken during remission. This technique may be applied to the treatment of other forms of leukemia, to other HLA types and potentially to other tumors as well.

Thus, to develop effective leukemia-specific immunotherapies, Pr3 and MPO were investigated as tissue-restricted proteins and it was found that the HLA-A2.1-restricted self-peptides, PR1 and MY4, derived from Pr3 and MPO, respectively, can be used to elicit peptide-specific CTL that preferentially attack myeloid leukemia based on aberrant expression of the parent proteins in the target cells. By using tetramers to study post-transplant and postinterferon treated patients for peptide-specific immunity, PR1 was established as a leukemia-associated antigen.

The data suggest that PR1 and MY4 could be used as target antigens to stimulate both active and passive leukemia-specific immunity. MY4-specific CTL will be given in an adoptive immunotherapy study with nonmyeloablative stem cell transplant, and in a clinical phase I trial other peptide antigens that are identified will be added to this approach. MY4-specific CTL will be selected and expanded ex vivo with the MY4 antigen for the production of leukemia-reactive CTL to produce a GVL effect and minimize GVHD.

A deductive strategy to identify peptide antigens from myeloperoxidase that are restricted to common HLA alleles that can be used to elicit leukemia-specific CTL responses in vitro. It has been shown, as previously discussed, in 9 HLA-A2+ AML patients who received NST that, in addition to PR1, MY4 is another new potential LAA. However, only 48% of the U.S. population has the HLA-A2 allele. Therefore, to extend any resulting immunotherapy strategies based on newly identified peptide antigens, whether each of the peptides predicted to have high binding (dissociation half-time >10) to the HLA-A3 and the HLA-B7 alleles can also elicit peptide-specific CTL will be addressed.

It will be determined whether it is possible to elicit CTL immunity against the remaining five peptides in Table 14 that are predicted to bind to HLA-A2.1. In addition, whether CTL against the predicted HLA-A3-, and HLA-B7-restricted epitopes from MPO can be elicited will be determined. Using the same method used to deduce HLA-A2-restricted peptides, several more peptides that are predicted to bind with high affinity to their respective HLA alleles have been identified. These peptides are shown in Table 16 and Table 17. As shown previously, peptides with predicted disassociation half-times of >10 are the most likely to bind to the relevant HLA alleles (Molldrem et al., 1996). Therefore, all 20 of the A3- and B7-restricted peptides will be synthesized and examined.

All peptides will be synthesized to a minimum of 95% purity. The peptides will be dissolved in a minimum of DMSO and solubility characteristics will be noted. Binding of each of the HLA-A2.1-restricted peptides will be performed using wild type T2 cells using the method of determining HLA-A2 stabilization by indirect fluorescence and flow cytometry. Relative fluorescence will be obtained using the BB7.2 hybridoma (ATCC) and compared to peptides with known binding characteristics, PR1 and pp65.

TABLE 16

MPO peptides predicted to bind HLA-A3 HLA-A3
Peptide Start (aa) Subsequence Residue
Half-Time Position Disassociation

| | | | | |
|---|---|---|---|---|
| | Starting | | SEQ | Predicted Binding |
| Peptide | Amino Acid | | ID | Half-Time |
| Number | Position | Sequence | NO | Dissociation |

| | | | | |
|---|---|---|---|---|
| 1 | 466 | VLGPTAMRK | 42 | 60.000 |
| 2 | 595 | GLPGYNAWR | 43 | 54.000 |
| 3 | 503 | TLIQPFMFR | 44 | 54.000 |
| 4 | 571 | RLFEQVMRI | 30 | 27.000 |
| 5 | 62 | VLSSMEEAK | 45 | 20.000 |
| 6 | 508 | FMFRLDNRY | 46 | 20.000 |
| 7 | 471 | AMRKYLPTY | 47 | 18.000 |
| 8 | 452 | AMVQIITYR | 48 | 13.500 |
| 9 | 663 | CIIGTQFRK | 49 | 13.500 |
| 10 | 361 | GLLAVNQRF | 50 | 13.500 |

To determine whether the remaining peptides from Tables 16 and 17 will also bind to HLA, the HLA-A3 and HLA-B7 alleles from EBV-transformed B cells derived from HLA-A3+ and HLA-B7+ normal donors were first cloned. These genes were inserted into the BirA-containing cassette that was used to construct the HLA-A2.1 tetramers and were then used to fold HLA-A3 and HLA-B7 tetramers using peptides with known high binding affinity to the respective alleles. Tetramers folded with the newly identified peptides will be used as reagents to test whether patients have evidence of circulating peptide-specific CTL.

The peptide-specific CTL lines generated in vitro from healthy donors that show peptide-specific lysis will be used as “reagents” to confirm the specificity of the tetramers. The A3 and B7 alleles have also been cloned into a mammalian vector containing the CMV promoter (Clonetech). Electroporation will be used to transduce T2 cells with these vectors. The transduced T2 cells will then be expanded for up to 1 month and sort-purified using the MoFlo high-speed cell sorter based on increased A3 or B7 surface expression after the addition of stabilizing A3- and B7-binding peptides.

The resulting T2 cells can then be used to determine whether the predicted peptides from Tables 16 and 17 bind to A3 and B7 by using A3- and B7-specific monoclonal antibodies (Immunotech and Pharmingen), and measuring surface expression. These binding results will be compared to peptides with known binding affinities to A3 and B7, such as influenza matrix and CMV pp65-derived peptides. The relative binding affinities of these peptides to the HLA allele will be determined by serial dilutions of each peptide and comparing them to PR1 after analyzing for surface HLA expression by flow cytometry. In this way, an IC₅₀ value will be determined for each peptide.

Using the methods described for PR1 and the first 5 MPO peptides examined, the resulting peptide-elicited CTL lines will be characterized for their ability to kill peptide-coated T2 cells, fresh leukemia cells and established leukemia cell lines such as U937 and K562. HLA restriction will be confirmed using targets without the relevant allele and by blocking experiments with antibodies specific for the relevant alleles. The amount of target cell killing will be determined using a standard 4-hr assay (Molldrem et al., 1996; Hensel et al., 1999) and will be correlated with target antigen expression and surface phenotype of the leukemia cells and healthy donor BM cells.

TABLE 17

MPO peptides predicted to bind HLA-B7 HLA-B7
Peptide Start (aa) Subsequence Residue
Half-Time Position Disassociation

| | | | | |
|---|---|---|---|---|
| | Starting | | SEQ | Predicted |
| Peptide | Amino Acid | | ID | Half-Time |
| Number | Position | Sequence | NO | Dissociation |

| | | | | |
|---|---|---|---|---|
| 1 | 434 | NPRWDGERL | 51 | 800.000 |
| 2 | 234 | IVRFPTDQL | 52 | 300.000 |
| 3 | 468 | GPTAMRKYL | 53 | 120.000 |
| 4 | 136 | RPFNVTDVL | 54 | 80.000 |
| 5 | 575 | QVMRIGLDL | 55 | 60.000 |
| 6 | 522 | NPRVPLSRV | 56 | 60.000 |
| 7 | 588 | MQRSRDHGL | 57 | 40.000 |
| 8 | 191 | SNRAFVRWL | 58 | 40.000 |
| 9 | 325 | TIRNQINAL | 59 | 40.000 |
| 10 | 352 | NLRNMSNQL | 60 | 40.000 |

MPO intracellular protein expression will be determined using direct intracellular FACS containing for the MPO protein with a FITC-labeled murine monoclonal antibody. This intracellular stain will be combined with surfaced antibodies for myeloid differentiation markers such as CD34, CD33, CD13, CD14, CD16 and HLA-DR to determine which stage of differentiation might be more susceptible to CTL killing. The MoFlo cytometer is capable of simultaneous 10-color analysis, which will greatly facilitate the analysis of progenitor stage of development. Both BM and PBMC will be examined similarly for MPO expression and surface phenotype and compared to determine whether there are differences in target susceptibility based on location (marrow vs. peripheral blood).

To determine whether CTL with specificity for myeloperoxidase-derived peptides can be detected in vivo in patients at diagnosis, before and after NST and after treatment with chemotherapy. Because CTL lines against both MY2 and MY4 could be elicited from normal donors and kill AML cells, it was next determined whether it was possible to detect these CTL in the peripheral blood of patients with AML. In contrast to CTL with specificity for MY4, MY2-specific CTL caused lysis of both leukemic and healthy bone marrow cells, which suggested it would be unlikely to find high circulating numbers of MY2-specific CTL since these might mediate autoimmunity in addition to anti-leukemia immunity. Previous studies using peptide/HLA-A2 tetramers to identify the similarly deduced peptide PR1 as an HLAA2.1-restricted leukemia associated antigen (LAA) (Molldrem et al., 2000) argued strongly that the same methodology could be used to determine whether MY2 and MY4 were also potential LAAs. Thus, paired samples from 90 μML patients treated using nonmyeloablative transplant regimens (up to 30 per year for the first 3 years) will be examined. Patients will be studied for immunity to the 6 peptides discussed, in addition to any peptide antigens that are found to elicit anti-leukemia immunity. This will include peptides with HLA-A3 and HLA-B7 restrictions. Patient peripheral blood samples will be obtained prior to transplant and then weekly after transplant, beginning on day 10 and continuing until day 100. Patient samples will then be examined at each follow-up in the BMT clinic, which will be monthly until 1 year post-transplant. PBMC samples will also be obtained from the donor pre-transplant. BM cells will be obtained from the donor if BM is used as the graft, and from the recipient prior to transplant and again on days 30, 100 and day 365 post-transplant. The PBMC and BM samples will be cryopreserved. PBMC samples will be used for later evaluation as more peptides are identified as potential LAAs (as discussed herein). The lymphocytes for surface expression of several markers, will be examined including CD3, CD4, CD8, CD16+56, CD45RA, CD45RO, CD57, CD28, CD27 as well as tetramer staining.

The maximum number of tetramer+ cells during the time course of study will be determined. Prior experience with viral antigen-specific CTL, with HA-1-specific CTL and with PR1-specific CTL suggest that the peak number of tetramer+ cells occurs over a 3 to 4 week period and often coincides (or may lag by a week or two) with the time of documented remission. Furthermore, the peak for other peptide-specific CTL is usually in the range of 1% to 10%. Significant increases over baseline of tetramer+ cells occurring during the study period will be verified. The study size will provide 85% power to establish a mean absolute increase of 1 percentage point (SD=3.5% based on previous data, type I error=0.05) at peak. In addition, the proportion of patients who show tetramer positivity, defined as at least 1% of cells positive at peak (SD±10% under assumed rate of 50% positivity) will be estimated. BM cells will be studied for MPO expression using intracytoplasmic staining combined with surface phenotypic makers that will allow the determination of the point of maturation of the BM cells. Expression of CD11a, CD13, CD14, CD16, CD33, CD34, CD80, CD86, HLA-ABC and HLA-DR will be examined. Because MY4 and the other peptides in this study are self-antigens, it is possible that AML patients treated with chemotherapy alone may have circulating numbers of MY4/MHC tetramer+ cells based on a previous study of CML patients using the PR1/HLA-A2 tetramer, however, this would seem unlikely. If these peptides are detected in AML patients that are in remission it may indicate that post-chemotherapy recovery of immunity is important for obtaining remission and the length of remission duration. Blood samples will be obtained from 10 consecutive AML patients receiving chemotherapy as alternative to NST, with samples obtained at the same time points after start of therapy as for the NST group. Thus, whether the proportion of patients showing tetramer positivity differs between the chemotherapy and NST groups will be addressed. It is anticipated that less than 5% of chemotherapy patients will show positivity compared to about 50% of NST patients. A chi-square test comparing the two proportions will have 92% power to detect the difference of interest (type I error=0.05), comparing the 10 chemotherapy patients to 90 NST patients. To determine the significance of circulating tetramer+ cells, it will also be assessed whether the presence of MY4 tetramer+ lymphocytes is associated with duration of disease response. All or nearly all of the 90 patients who start NST will be in complete remission or achieve it following NST therapy. From 30 to 40 relapses will have occurred at the time of data analysis, one year after the last patient is treated. Patients will be classified into two groups based on whether or not the patients have detectable tetramer+ cells. The association of response duration to tetramer positivity status will be modeled assuming proportional hazards. The study is powered to detect a tripling in risk of relapse associated with failure to detect tetramer+ lymphocytes. In addition, separate assessments of the association of tetramer positivity with duration of response determined by molecular and cytogenetic methods, will be made. It will also be important to determine whether the MY4-CTL are functional. Various methods of assessing function have been described, including cytokine secretion, CD69 upregulation, cell proliferation, and cytotoxicity. Tetramer staining and cytokine flow cytometry (CFC) will simultaneously be determined on all patients since the MoFlo will greatly facilitate these experiments. PE-labeled antibody to gamma-interferon and PECy7-labeled tetramers will be used to in these studies. Cells will first be labeled for 10 min at 37° C. with tetramer and FITC-labeled CD8 and then stimulated with MY4 peptide at 2 μg/ml. Brefeldin A will be added during culture to inhibit secretion of cytokines, and after 6 hr cells will be permeabilized and stained for interferon. This technique has been successfully used to monitor PR1-CTL responses after vaccination and it was found that CFC positivity correlates with cytotoxicity. In select patients with sufficient numbers of available PBMC and tetramer+ cells, the tetramer+ population will be purified by high-speed sorting using the MoFlo cytometer. Both the tetramer+ and tetramer-cells will be tested for cytotoxicity against cryopreserved leukemia targets prior to NST or chemotherapy. Because the MoFlo is capable of 4-way simultaneous sorting, the killing of peptide-coated target cells of the MY4-CTL will be compared to other peptide-specific CTL (i.e. pp65 in serpositive patients) to directly compare lytic potential.

To determine the optimal method to select and expand peptide-specific CTL from healthy donors that can be used for adoptive cellular immunotherapy of NST recipients. Because CTL with myeloid self-antigen specificity are present at very low precursor frequencies (Molldrem et al., 1999), these CTL will need to be expanded before they could be used as part of an adoptive transfer immunotherapy strategy. While there are currently some commercially available methods for the separation of antigen-specific CTL based on cytokine secretion, they are presently not licensed for use in patients, and the best method to separate antigen-specific CTL has not been defined. Similarly, the optimal method to expand CTL in short-term culture conditions is not yet defined, and expansions on large scale without the use of fetal calf serum remain a major obstacle to cellular immunotherapy. Two general expansion methods to elicit MY4-CTL in vitro will be compared.

In the first approach, peptide-pulsed dendritic cells (DCs) will be used to stimulate responder PBMC weekly for 4 to 6 wk. Although there are many potential sources for precursors to mature DC, including CD34+ cell-derived hematopoietic precursors, monocyte-derived DC have been chosen because they are more readily available in large numbers from donor leukapheresis products. For practical reasons, this methodology is most likely to yield the greatest potential number of DC, which will be needed to grow low precursor frequency self-antigen specific CTL. For similar practical reasons, the use of interferon (IFN) and GM-CSF to grow DC, with or without TNF-cc added to mature the cells during the last 48 to 72 hr of culture have been studied. The advantage is that both IFN and GMCSF are commercially available and have been used extensively in humans. In brief, DCs were grown using combinations of either 1,000 U/ml IL-4 plus 500 U/ml GM-CSF (termed DC/4GM) or 1,000 U/ml interferon-α2b plus 500 U/ml GM-CSF (termed DC/IGM). Previously cryopreserved PBMC from HLA-A2+healthy donors were thawed, washed and adhered to plastic flasks prior to the addition of media +10% human serum (HS) with the addition of the above cytokines. T2 cells were maintained in RPMI+10% HS prior to co-culture with donor PBMC. At the end of 7 days, DC were pulsed with 20 μg/ml PR1 peptide, irradiated and combined with fresh PBMC from the same donor at a 1:2 ratio. On day 7, the culture was restimulated with PR1-pulsed DC (or T2) and on day 8 IL-2 at 20 U/ml was added to the cultures. Restimulation and IL-2 addition was repeated weekly until day 26 through 28 when the PR1-CTL cultures were tested for their ability to lyse PR1-coated target cells or CML cells. The PR1-CTL were also evaluated for surface phenotype with the PR1/HLA-A2 tetramer and anti-CD8. In general, both DC/IGM and T2 cells elicited PR1-CTL. However, T2 were nearly twice as efficient, typically yielding 3% to 6% PR1-CTL (of the bulk culture, determined by tetramer staining) by 4 wk and DC/IGM yielding only 0.5% to 2% PR1-CTL. Since T2 cells may be difficult to use clinically for regulatory reasons, alternative sources of stimulators must be studied.

This procedure is discussed in detail herein and in Molldrem et al. (2003); (2002); (1999); (1998); (1997); each incorporated herein in their entirety by reference). Typical yields of antigen-specific tetramer+cell numbers were 3% to 5% PR1-CTL using AAPC or T2 cells, but only 0.3% to 2% when DC/IGM are used. These observations are likely to apply to MY4, but all of the comparative experiments will be repeated using this peptide. In addition, serum-free growth conditions will be compared to 10% HS and 5% albumin as a serum substitute in both the DC cultures and the CTL cultures. This will determine the optimal conditions that produce the highest numbers of functional MY4-CTL in the shortest period of time. Weekly tetramer staining during bulk culture restimulations will be used to compare numbers of MY4-CTL, and CFC and cytotoxicity experiments will be used to compare the effector function of the cells.

Additionally, two methods to select antigen-specific CTL from bulk culture will be compared. Although it is predicted that the bulk culture conditions described above will yield CTL that, when adoptively transferred to patients, will not produce significant GVHD, it is possible that GVHD occurs due to the non-specific CTL remaining in the bulk cultures. Since CTL cloning at the end bulk culture is time-consuming and would therefore need to be performed prophylactically in each patient, and because the transfer of CTL clones is not likely to yield long-lived CTL in vivo (Riddell and greenberg, 1994; ridden and Greenberg, 1995a; Riddell and Greenberg, 1995b), it is suggested that separation of the peptide-specific fraction from the bulk culture may improve the purity and thereby reduce GVHD. In the first approach to select the antigen-specific CTL, cytokine capture microbead technology developed by Miltenyi Inc., which relies on the ability of antigen-specific CTL to secrete IFN after antigen challenge will be used. These reagents are commercially available, although they are not yet approved for clinical use. MY4-CTL obtained after 4 wk of weekly restimulation will be incubated with MY4 antigen and the bi-specific antibody with anti-CD45 and anti-IFN binding will be co-incubated with the cells. A secondary antibody with anti-IFN antibody that is directly linked to microbeads (supplied by Miltenyi, Inc., Germany) will then be used to select out IFN-secreting CTL from bulk culture. In the second approach, the IFN capture method will be compared to a modified peptide/MHC-conjugated bead method developed to select antigen-specific CTL. In previous studies using the PR1 peptide, the feasibility of using PR1 peptide/HLA-A2 monomers linked to streptavidin-coated microbeads (Miltenyi Inc, Germany) to separate antigen-specific CTL from bulk cultures of mixed lymphocytes was demonstrated as described herein (see also Molldrem et al. (2003); (2002); (1999); (1998); (1997), each incorporated by reference herein in their entirety). These studies will be repeated using MY4/HLA-A2 monomers to determine whether similar yields of antigen-specific CTL can be obtained. This methodology will be compared to the standard IFN-capture methodology developed by Miltenyi. Although it is reasonable to expect that the commercial technology will produce sufficient purity of CTL, the device may not capture all CTL with the potential to recognize the cognate peptide/MHC ligand. Since it is unclear whether non-secreting antigen-specific CTL might be required to maintain the more functional fraction in vivo, or whether the non-secreting CTL later become able to express effector function, too few CTL may be selected using the commercial product. Monomer coated beads are likely to capture all of the available antigen-specific CTL from the bulk culture, as shown in previous studies.

To determine whether peptide antigen-specific CTL can be adoptively transferred to myeloid leukemia patients after T cell-depleted NST to facilitate engraftment, boost GVL and reduce GVHD. The overall treatment strategy to enhance GVL and reduce GVHD is to use a nonablative preparative regimen to achieve engraftment of allogeneic blood stem cell or bone marrow transplant and to be the platform to deliver antigen-specific CTL therapy. The goal is to determine the maximal dose of MY4-specific CTL that will provide engraftment in >80% of patients while preventing development of acute GVHD (the baseline rate of grade 3 and 4 acute GVHD <15%) and mediating a GVL effect. T-cells have been shown to facilitate engraftment and are required to prevent rejection with the established nonablative preparative regimens. Recently, the use of a nonablative preparative regimen, including fludarabine and melphalan, was demostrated that successfully allowed engraftment of an allogeneic blood stem cell or bone marrow transplant from HLA identical siblings or matched unrelated donors in patients with AML and other hematologic malignancies. This approach allows allogeneic transplantation for patients who were elderly or had co-morbidities, which would make them ineligible for high dose myeloablative regimens. Acute GVHD is a major cause of morbidity and mortality and treatment for GVHD remains unsatisfactory, particularly in older and infirm patients. Thus, this study extends this therapeutic strategy, with manipulation of donor cells to reduce alloreactive cells by enriching for MY4-CTL (and other antigen-specific CTL determined previously). This will be accomplished by selecting and adoptively transferring the MY4-CTL. These cells thus, would not be expected to react against the recipient tissues, but would still be able to mediate anti-leukemia effects. Patients with AML who have an HLA compatible related or unrelated donor who failed to respond to initial chemotherapy or after relapse will be used. Lymphocytes will be collected from the donor by apheresis with a goal of collecting 2×10⁸ CD3+ T-cells/kg. Donor type peripheral-blood mononuclear cells are plated in 75-mm flasks at 2×10⁶ cells/ml and stimulated with 2×10⁶ irradiated donor-derived DC from the patient's donor.

If other methods, as discussed above, produce superior results, the procedure will be modified to incorporate those methods. CTL bulk cultures will be restimulated weekly, as described herein. After 28 days of co-culture, the cells will be harvested and live cells will be isolated on Ficoll gradient. The cultures are treated with IL-2 20 U/mL final concentration on day 8 and weekly thereafter. All the responders and stimulators are suspended into RPMI-1640 medium with 2 mM L-glutamine, 100 U/mL penicillin, 0.1 mg/mL streptomycin, 25 mM Hepes, 1 mM sodium pyruvate, 0.1 mM non-essential amino acids and 5×10⁵ M 2-mercaptoethanol. Autologous serum at 10% will be supplemented. This will be modified if the results show that albumin or serum-free conditions can be substituted. Stem cells will be collected from the donor's blood after G-CSF administration according to standard procedures. If there is a medical contraindication to G-CSF administration, stem cells will be collected by bone marrow harvest. Apheresis will occur daily for up to a total of four procedures in order to collect >5×10⁶ CD34+ cells/kg recipient weight after positive selection of CD34+ cells. The minimal acceptable number is 2×10⁶ CD34+ cells/kg recipient weight. Cells will undergo CD34 selection using the Isolex or Miltenyi device with the final composition including >2×10⁶ CD34+ cells and <1×10⁵ T-cells/kg. The apheresis product from each day will be cryopreserved according to standard procedures. For the preparative regimen, patients will receive fludarabine 25 mg/m² intravenously daily at the same time over 30 min on days 6, 5, 4, 3, 2 and melphalan 70 mg/m² on day 3 and 2 administered following completion of the fludarabine. The allogeneic hematopoietic cell infusion consisting of >2×10⁶ CD34+ cells plus MY4-CTL is administered on day 0. MY4-CTL will be administered in a phase I study to define the maximal dose that can be administered without producing GVHD. Tetramer staining will be performed at the end of bulk culture.

Dose levels will be 1: 5×10⁷ CD8+ cells/kg; 2: 1×108 CD8+ cells/kg and 3: 5×10⁸ CD8+ cells/kg. Patients will not receive post transplant immunosuppressive therapy or growth factors, but will receive standard supportive care for prevention of infection. It is predicted, based upon previous results, that current expansion techniques will yield sufficient MY4-CTL from the collection of 2×10⁸ CD3+ T-cells/kg. Patients will be assessed for engraftment and chimerism on days 28, 56, 90, 180, 365 and yearly post transplant. Chimerism will be assessed by microsatellite STR (short tandem repeat) markers and analyzed using GeneScan software on peripheral blood, assaying both T-cells and myeloid cells separately. Engraftment is defined as documentation of >50% donor derived T-cells on day 90. Disease remission will be assessed by bone marrow evaluation on day 28, 90 and 180 and as indicated thereafter. Acute and chronic GVHD will be assessed by standard criteria. Peripheral blood will be collected weekly for tetramer staining and for surface phenotyping, as performed above.

Evaluation for Donor Leukocyte Infusion. If no GVHD is observed, and if antigen-specific CTL are no longer detectable, patients will be evaluated at day 45 for infusion of antigen specific T cells. Patients in remission and with evidence of T cell chimerism (>5% recipient) will be eligible for donor cell infusion. The same cell dose will be transferred. Primary endpoints are the achievement of durable engraftment defined as the durable presence of >50% donor T-cells and myeloid cells (in the absence of leukemia relapse) and development of grade 3 acute GVHD using standard criteria. Secondary endpoints include relapse of AML, non-relapse mortality and survival. The non-alloreactive CTL are intended to augment engraftment without inducing GVHD in the absence of leukemia relapse. Patients will be treated in cohorts of 6 patients. The starting dose level is level one. If graft failure occurs in 2 of a given group without GVHD, the dose will be considered too low to ensure engraftment and subsequent patients will be entered at the next higher dose level. If grade 3 acute GVHD does not occur in a given group, subsequent patients will receive the next higher dose level. If grade 3 acute GVHD occurs in one patient, 6 additional patients will be treated at a given level. If grade 3 acute GVHD occurs in 2 patients (out of a maximum of 12) at a given level, the rate of GVHD will be considered excessive and that level will be terminated. The MTD is the level, which allows engraftment in at least 5 of 6 patients without excessive GVHD. Ten additional patients will be treated at the MTD. Early stopping criteria will be established to close the study in the event of excess death or relapse. A phase I trial will be conducted in which a maximum of 60 patients will be assigned among the three levels of cell doses. Outcomes to be monitored are immune response and occurrence of GVHD, both defined over a 100-day period from receipt of transplant. Within each dose group, the goals are to achieve a rate of at least 20% of patients with immune response while maintaining at most a 20% rate of GVHD. Patient outcomes will be monitored and compared to stopping boundaries computed using a Bayesian method. Priors will be based on results of the PR1 peptide vaccine trial of active immunotherapy, and doses are assumed independent. The vaccine trial has shown a peak tetramer response of 2% of all CD8+ T-cells, which will provide a good starting estimate for the design of a passive adoptive transfer cell therapy protocol. Stopping boundaries will be computed using available software. This is intented to terminate a dose level if the computed probability of achieving at least a 20% immune response rate based on accumulated data is less than 10%, or the probability of 20% GVHD rate is at least 90%. Among dose levels not terminated early, the dose level having the highest observed immune response rate will be declared optimal, unless there are major differences in GVHD rates.

It is expect, based on previous data, that therapy based on peptides derived from self hematopoietic proteins will lead to advances in the treatment of myeloid leukemia. Peptide-specific adoptive T-cell immunotherapy, combined with CTL specific for other MPO epitopes may enhance the efficacy of such an approach. If the clinical study fails to convey an anti-leukemia immune response with adoptive transfer of MY4-CTL, studies involving adoptive transfer of CTL other MPO- or Pr3-derived peptides may be conducted. In addition, the use of the HLA-A2.1-transgenic mouse model may allow for investigation into mechanisms of tolerance toward MPO peptides such as, but not limited to, MY4. In addition, the possibility that the MY4/HLA-A2 tetramer could be used to select for a homogeneous population of leukemia-reactive CTL might allow for the first time the adoptive transfer of CTL across MHC barriers to treat leukemia (Toes et al., 1996). The results as disclosed herein, show the development of tolerance toward MY4.

### Example 21

### Cyclin E1

To search for other potential tumor-associated antigens, the cyclin E family of proteins were investigated, because it is well known that cyclin E is constitutively expressed in some tumor cells in dependent of the cell cycle, and aberrantly expressing cyclin E contributes to tumorigenesis as a result of chromosomal instability. Cyclin E2 is a homologue of cyclin E1 and both proteins have restricted tissue distribution. To investigate whether cyclin E1 and E2 are over-expressed in hematological malignancy, cyclin E1 and cyclin E2 mRNA expression were first analyzed in 21 patients with hematological malignancy (11 CML (CP), 5 CML (BC), 2 μML, 2 ALL, 1 NHL) and 12 normal donors by RT-PCR. PBMCs from 15 patients and 5 normal donors expressed cyclin E1 mRNA. The relative expression level of cyclin E1 mRNA standardized to β-actin was higher in patients than in normal donors (p =0.0149). Cyclin E2 mRNA was highly over-expressed in PBMCs of patients (10/21) as compared to normal donor PBMCs (0/12; p=0.0109). Next cyclin E1 protein expression was analyzed in 18 of 21 patients with hematological malignancy and 3 of 12 normal donors by western blotting. Although none of PBMCs from normal donor expressed cyclin E1 protein, except one CML (CP) patient, almost all of PBMCs from patients expressed cyclin E1 protein even if they did not express mRNA at the same time point.

Nonameric peptides derived from cyclin E1 and cyclin E2 and predicted to bind to the HLA-A2 allele have similar amino acid sequences, differing only at position 7. Thus, the binding of CCNE1₁₄₄₋₁₅₂ (cyclin E1 derived) and CCNE2₁₄₄₋₁₅₂ (cyclin E2 derived) was compared to that of PR1 by a peptide-binding assay. The ability of CCNE1₁₄₄₋₁₅₂ and CCNE2₁₄₄₋₁₅₂ to stabilize HLA-A2 on the surface of T2 cells was almost the same as that of PR1 (FI CCNE1₁₄₄₋₁₅₂/FI PR1=1.008, FI CCNE2₁₄₄₋₁₅₂/FI PR1=1.189). To determine whether CCNE1₁₄₄₋₁₅₂ and CCNE2₁₄₄₋₁₅₂ specific CTLs could be elicited in vitro, PBMCs from 7 HLA-A2 positive normal donors were stimulated with peptide-pulsed T2 cells. In 3 of 7 donors, CCNE1₁₄₄₋₁₅₂-stimulated CTL lines killed both CCNE1₁₄₄₋₁₅₂ and CCNE 2₁₄₄₋₁₅₂-pulsed T2 cells but not non-peptide-pulsed T2 cells and irrelevant peptide-pulsed T2 cells. In 4 of 7 same donors, CCNE2₁₄₄₋₁₅₂-stimulated CTL lines killed both CCNE2₁₄₄₋₁₅₂ and CCNE1₁₄₄₋₁₅₂-pulsed T2 cells but not non-peptide-pulsed T2 cells and irrelevant peptide-pulsed T2 cells.

Thus, each peptide-specific CTLs can recognize both peptides with HLA-A2, but the immunogenicity of each peptide is different between individuals. Moreover, one of 4 CCNE1₁₄₄₋₁₅₂-stimulated CTL lines which killed CCNE1₁₄₄₋₁₅₂-pulsed T2 cell killed HLA-A2 transfected K562 leukemic cell line which is over-expressing cyclin E1 protein, but not non-transfected K562 leukemic cell line. Thus, CCNE1₁₄₄₋₁₅₂-specific CTL can distinguish leukemic cell lines from normal PBMCs. From the data, it was concluded that cyclin E1/E2 derived peptides are potential tumor-antigens, because 1) cyclin E1/E2 are highly over-expressed in hematological malignancy, 2) cyclin E1/E2 peptides can sufficiently bind to HLA-A2 to stimulate CTL and 3) CCLE1₁₄₄₋₁₅₂ specific CTL preferentially kills leukemic cell lines HLA-A2 restrictively.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### XIII. REFERENCES

The following references, to the extent that they provide exemplary procedural or other details supplementary to those set forth herein, are specifically incorporated herein by reference.
U.S. Pat. No. 4,196,265
U.S. Pat. No. 4,435,386
U.S. Pat. No. 4,436,727
U.S. Pat. No. 4,436,728
U.S. Pat. No. 4,505,899
U.S. Pat. No. 4,505,900
U.S. Pat. No. 4,520,019
U.S. Pat. No. 4,554,101
U.S. Pat. No. 4,579,945
U.S. Pat. No. 4,684,611
U.S. Pat. No. 4,797,368
U.S. Pat. No. 4,866,034
U.S. Pat. No. 4,877,611
U.S. Pat. No. 4,879,236
U.S. Pat. No. 4,950,645
U.S. Pat. No. 4,952,500
U.S. Pat. No. 5,139,941
U.S. Pat. No. 5,302,523
U.S. Pat. No. 5,322,783
U.S. Pat. No. 5,384,253
U.S. Pat. No. 5,399,363
U.S. Pat. No. 5,464,765
U.S. Pat. No. 5,466,468
U.S. Pat. No. 5,538,877
U.S. Pat. No. 5,538,880
U.S. Pat. No. 5,543,158
U.S. Pat. No. 5,550,318
U.S. Pat. No. 5,563,055
U.S. Pat. No. 5,580,859
U.S. Pat. No. 5,589,466
U.S. Pat. No. 5,609,870
U.S. Pat. No. 5,610,042
U.S. Pat. No. 5,641,515
U.S. Pat. No. 5,656,610
U.S. Pat. No. 5,693,762
U.S. Pat. No. 5,702,932
U.S. Pat. No. 5,736,524
U.S. Pat. No. 5,739,169
U.S. Pat. No. 5,780,448
U.S. Pat. No. 5,785,970
U.S. Pat. No. 5,789,215
U.S. Pat. No. 5,801,005
U.S. Pat. No. 5,824,311
U.S. Pat. No. 5,830,880
U.S. Pat. No. 5,846,225
U.S. Pat. No. 5,846,233
U.S. Pat. No. 5,846,945
U.S. Pat. No. 5,861,155
U.S. Pat. No. 5,871,986
U.S. Pat. No. 5,945,100
U.S. Pat. No. 5,980,912
U.S. Pat. No. 5,981,274
U.S. Pat. No. 5,994,136
U.S. Pat. No. 5,994,624
U.S. Pat. No. 6,013,516
U.S. Pat. No. 6,020,192
U.S. Pat. No. 6,027,727
U.S. Pat. No. 6,054,297
Aichele et al., J. Exp. Med., 182:261-266, 1995.
Alam et al., Nature, 381:616-620, 1996.
Alexander-Miller et al., J. Exp. Med., 184:485-492, 1996.
Alexander-Miller et al., J. Exp. Med., 188:1391-1399, 1998.
Alexander-Miller et al., Proc. Natl. Acad. Sci. USA, 93:4102-4107, 1996.
Altman et al., Science, 274:94-96, 1996.
Anderton et al., J. Exp. Med., 193:1-11, 2001.
Antin, Blood, 82:2273-2277, 1993.
Arap et al., Cancer Res., 55(6):1351-1354, 1995.
Austin-Ward and Villaseca, Rev. Med. Chil., 126(7):838-45, 1998.
Azuma et al., Cell Immunol., 116(1):123-134, 1988.
Baichwal and Sugden, In: Gene Transfer, Kucherlapati (Ed.), NY, Plenum Press, 117-148, 1986.
Bajorin et al., J. Clin. Oncol., 6(5):786-92, 1988.
Bakhshi et al., Cell, 41(3):899-906, 1985.
Ballieux et al., Clin. Exp. Immunol., 100:186-193, 1995.
Barrett et al., Blood, 95:3323-3327, 2000.
Barrett et al., Bone Marrow Transplant., 21:543-551, 1998.
Behre et al., Methods, 17:231-237, 1999.
Benjamini et al., Adv. Exp. Med. Biol., 303:71-77, 1991.
Bernhard etal., Cancer Res., 55:1099-1104, 1995.
Blomer et al., J. Virol., 71(9):6641-6649, 1997.
Bocchia et al., Blood, 85:2680-2684, 1995.
Bocchia et al., Blood, 87:3587-3592, 1996.
Boon et al., Immunol Today, 18:267-268, 1997.
0Boon et al., Important Adv. Oncol., 53-69, 1994.
Bories et al., Cell, 59:959, 1989.
Borregaard and Cowland, Blood, 89:3503-3521, 1997.
Borrello et al., Blood, 95:3011-3019, 2000.
Bourdette et al., J. Immunol., 152:2510-2519, 1994.
Boussiotis et al., J. Exp. Med., 184:365-376, 1996.
Boussiotis et al., Res. Immunol., 146:140-149, 1995.
Braunschweig et al., Blood, 96:3291, 2000.
Brouwer et al., Clin. Exp. Immunol., 98:448-453, 1994.
Brutlag et al., Comput. Appl. Biosci., 6(3):237-245, 1990.
Bukowski et al., Clin. Cancer Res., 4(10):2337-47, 1998.
Burchert et al., Blood, 02:659, 2002.
Caldas et al., Nat. Genet., 8(1):27-32, 1994.
Campanelli et al., J. Exp. Med., 172:1709-1715, 1990.
Capaldi et al., Biochem. Biophys. Res. Comm., 74(2):425-433, 1977.
Celis et al., Proc. Natl. Acad. Sci. USA, 91:2105-2109, 1994.
Chen and Okayama, Mol. Cell Biol., 7(8):2745-2752, 1987.
Chen et al., Biochem. Biophys. Res. Commun., 200:1130-1135, 1994.
Cheng et al., Cancer Res., 54(21):5547-5551, 1994a.
Chou and Fasman, Adv. Enzymol., 47:45-148, 1978b.
Chou and Fasman, Annu. Rev. Biochem., 47:251-276, 1978a.
Chou and Fasman, Biochemistry, 13(2):211-222, 1974b.
Chou and Fasman, Biophys. J., 26(3):385-399, 1979.
Christodoulides et al., Microbiology, 144(Pt 11):3027-37, 1998.
Clark et al., Blood, 98:2887-2893, 2001.
Cleary and Sklar, Proc. Natl. Acad. Sci. USA, (21):7439-7443, 1985.
Cleary et al., J. Exp. Med., 164(1):315-320, 1986.
Collins, Jr. et al., J. Clin. Oncol., 15:433-444, 1997.
Cortes et al., Leukemia, 12:455-462, 1998.
Cotten et al., Proc. Natl. Acad. Sci. USA, 89(13):6094-6098, 1992.
Coupar et al., Gene, 68:1-10, 1988.
Culver et al., Science, 256(5063):1550-1552, 1992.
Curiel, Nat. Immun., 13(2-3):141-164, 1994.
Davidson et al., J. Immunother., 21(5):389-98, 1998.
den Haan et al., Science, 268:1476-1480, 1995.
den Haan et al., Science, 279:1054-1057, 1998.
Dengler et al., Br. J. Haematol., 89:250-257, 1995.
Dermime et al., Blood, 86, 1995.
Dermime et al., Bone Marrow Transplant., 19:989-999, 1997.
Devergie et al., Bone Marrow Transplant., 20:11-9, 1997.
Dickinson et al., Nat. Med., 8:410-414, 2002.
Dillman, Cancer Biother. Radiopharm., 14(1):5-10, 1999.
Dolstra et al., J. Immunol., 158:560-565, 1997.
Drobyski et al., Blood, 83:1980-1987, 1994.
Dudley et al., Science, 298:850-854, 2002.
Dunbar et al., Curr. Biol., 8:413-416, 1998.
Faber et al., Biol. Blood Marrow Transplant., 2:31-36, 1996.
Faber et al., Blood, 86:2821-2828, 1995.
Faber et al., J. Clin. Invest., 96:877-883, 1995.
Faderl et al., Ann. Intern. Med., 131:207-219, 1999.
Fechheimer et al., Proc. Natl. Acad. Sci. USA, 84:8463-8467, 1987.
Fetrow and Bryant, Biotechnology, 11(4):479-484, 1993.
Fraley et al., Proc. Natl. Acad. Sci. USA, 76:3348-3352, 1979.
Franssen et al., Kidney Int., 57:2195-1206, 2000.
Franssen et al., Lancet., 347:116, 1996.
Friedmann, Science, 244:1275-1281, 1989.
Gale et al., Ann. Intern. Med., 120:646-652, 1994.
Gallimore et al., J. Exp. Med., 187:1383-1393, 1998.
Gao et al., Blood, 95:2198-2203, 2000.
Garlie et al., J. Immunother., 22:336-345, 1999.
Gaschet et al., J. Clin. Invest., 98:100-107, 1996.
Gaugler et al., J. Exp. Med., 179:921-930, 1994.
Giralt and Kolb, Curr. Opin. Oncol., 8:96-102, 1996.
Gopal, Mol. Cell Biol., 5:1188-1190, 1985.
Goulmy et al., N. Engl. J Med., 334:281-285, 1996.
Graham and Van Der Eb, Virology, 52:456-467, 1973.
Grossman and Paul, Proc. Natl. Acad. Sci. USA, 89:10365-10369, 1992.
Grunhaus and Horwitz, Seminar in Virology, 3:237-252, 1992.
Hanibuchi et al., Int. J. Cancer, 78(4):480-485, 1998.
Harlan and Weintraub, J. Cell Biol., 101:1094-1099, 1985.
Harlow and Lane, Antibodies: A Laboratory Manual. Cold Spring Harbor Laboratory Press, Cold Spring harbor, N.Y., 553-612, 1988.
Hellstrand et al., Acta Oncol., 37(4):347-353, 1998.
Hensel et al., Bone Marrow Transplant., 23:71-78, 1999.
Hermonat and Muzycska, Proc. Natl. Acad. Sci. USA, 81:6466-6470, 1984.
Herr et al., J. Immunol. Methods, 191:131-142, 1996.
Herr et al., J. Immunol. Methods, 203:141-152, 1997.
Horowitz et al., Bone Marrow Transplant., 17(Suppl 3):S5-6, 1996.
Horwich et al., J. Virol., 64:642-650, 1990.
Hui and Hashimoto, Infect. Immun., 66(11):5329-36, 1998.
Husson et al., J. Bacteriol., 172(2):519-524, 1990.
Hussussian et al., Nat. Genet., 8(1):15-21, 1994.
Irie and Morton, Proc. Natl. Acad. Sci. USA, 83(22):8694-8698, 1986.
Irie et al., Lancet., 1(8641):786-787, 1989.
Jacobs et al., Nature, 327(6122):532-535, 1987.
Jameson and Wolf, Comput. Appl. Biosci., 4(1):181-186, 1988.
Janetzki et al., J. Immunol. Methods, 234:1-12, 2000.
Jennette et al., Semin. Diagn. Pathol., 18:3-13, 2001.
Johnson et al., In: Biotechnology And Pharmacy, Pezzuto et al. (Eds.), Chapman and Hall, NY, 1993.
Ju et al., J. Neuropathol. Exp. Neurol., 59(3):241-50, 2000.
Kaeppler et al., Plant Cell Reports, 9:415-418, 1990.
Kamb et al., Nat. Genet., 8(1):23-2, 1994.
Kamb et al., Science, 2674:436-440, 1994.
Kaneda et al., Science, 243:375-378, 1989.
Kato et al, J. Biol. Chem., 266:3361-3364, 1991.
Kelleher and Vos, Biotechniques, 17(6):1110-7, 1994.
Kerr et al., Br. J. Cancer, 26(4):239-257, 1972.
Klinman, Curr. Prot. Immunol., 6.19:18, 1994.
Kochenderfer and Molldrem, Curr. Oncol. Rep. 3:193-200, 2001.
Kochenderfer et al., Blood, 100:3639-3645, 2002.
Kolb and Holler, Curr. Opin. Oncol., 9:139-145, 1997.
Kolb et al., Blood, 86:2041-2050, 1995.
Kolb et al., Bone Marrow Transplant, 17:449-452, 1996.
Kwak et al., Proc. Natl. Acad. Sci. USA, 93:10972-10977, 1996.
Kyte and Doolittle, J. Mol. Biol., 57(1):105-32, 1982.
Lalvani et al., J. Exp. Med., 186:859-865, 1997.
Laughlin et al., J. Virol., 60(2):515-524, 1986.
Lebkowski et al., Mol. Cell. Biol., 8(10):3988-3996, 1988.
Lee et al., Nat. Med., 5:677-685, 1999.
Lehmann et al., Eur. J Immunol., 25:340-347, 1995.
Levine et al., J. Hematother., 7:437-448, 1998.
Lewin et al., Blood, 100:2235-2242, 2002.
Lotte et al., Adv. Tuberc. Res., 21:107-93; 194-245, 1984.
Lubbert et al., Leukemia, 13:1420-1427, 1999.
Luelmo, Am. Rev. Respir. Dis., 125(3 Pt 2):70-72, 1982.
Mackinnon et al., Bone Marrow Transplant., 15:591-594, 1995.
Maeurer et al., J. Clin. Invest., 98:1633-1641, 1996.
Mann et al., Cell, 33:153-159, 1983.
Marchand et al., Int. J. Cancer, 63:883-885, 1995.
Marijt et al., Blood 82:3778-3785, 1995.
Marincola et al., J. Immunother. Emphasis Tumor Immunol., 19:192-205, 1996.
Martin et al., Nature, 345(6277):739-743, 1990.
Matulonis et al., Blood, 85:2507-2515, 1995.
Mavroudis et al., Bone Marrow Transplant., 17:793-799, 1996.
Mayet et al., Eur. J. Clin. Invest., 27:893-899, 1997.
McLaughlin et al., J. Virol., 62(6):1963-1973, 1988.
Miller etal., Am. J. Clin. Oncol., 15(3):216-221, 1992.
Mitchell et al., Ann. NY Acad. Sci., 690:153-166, 1993.
Mitchell et al., J. Clin. Oncol., 8(5):856-869, 1990.
Molldrem et al., Blood, 88:2450-2457, 1996.
Molldrem et al., Blood, 90:1858, 1998.
Molldrem et al., Blood, 90:2529-2534, 1997.
Molldrem et al., Br. J. Haematol., 102:1314-1322, 1998.
Molldrem et al., Cancer Res., 59:2675-2681, 1999.
Molldrem et al., Curr. Opin. Hematol., 9:503-508, 2002.
Molldrem et al., J. Clin. Invest., 111:639-647, 2003.
Molldrem et al., Nat. Med., 6:1018-1023, 2000.
Molldrem et al., Oncogene, 21:8668-8673, 2002.
Morgan et al., J. Immunol., 160:643-651, 1998.
Mori et al., Cancer Res., 54(13):3396-3397, 1994.
Morton et al., Arch. Surg., 127:392-399, 1992.
Muller-Berat et al., Clin. Immunol. Immunopathol., 70:51-59, 1994.
Muzyczka, Curr. Topics Microbiol. Immunol., 158:97-129, 1992.
Nakamoto et al., J. Immunol., 158:5692-5697, 1997.
Naldini et al., Science, 272(5259):263-267, 1996.
Nanda and Sercarz, Cell, 82:13-17, 1995.
Nelson et al., Blood, 88:580-589, 1996.
Nestle et al., Nature Med., 4:328-332, 1998.
Nicolas and Rubinstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Nobri et al., Nature (London), 368:753-756, 1995.
Okamoto etal., Proc. Natl. Acad. Sci. USA, 1(23):11045-11049, 1994.
Omirulleh et al., Plant Mol. Biol., 21(3):415-28, 1993.
Orlow et al., Cancer Res, 54(11):2848-2851, 1994.
Papadopoulos et al., Blood, 90:4938-4946, 1997.
Pardoll, Current Opin. Immunol., 14:619-623, 1992.
Pardoll, Nature Rev. Immunol., 2:227-238, 2002.
Pardoll, Nature, 369:357, 1994.
Parker et al., J. Immunol., 152:163, 1994.
Parkhurst et al., J. Immunol., 157:2539-2548, 1996.
Paskind et al., Virology, 67:242-248, 1975.
PCT Appln, WO 91/16347
PCT Appln. WO 94/09699
PCT Appln. WO 95/06128
Pietras et al., Oncogene, 17(17):2235-49, 1998.
Pinilla-Ibarz et al., Blood, 95:1781-1787, 2000.
Potrykus et al., Mol. Gen. Genet., 199:183-188, 1985.
Qin et al., Proc. Natl. Acad. Sci. USA, 95(24): 14411-14416, 1998.
Rabinovich et al., Science, 265(5177):1401-1404, 1994.
Rammensee et al., Immunogenetics, 41:178-228, 1995.
Ravindranath and Morton, Intern. Rev. Immunol., 7: 303-329, 1991.
Rees et al., Proc. Natl. Acad. Sci. USA, 96:9781-9786, 1999.
Reich-Zeliger et al., Immunity, 13:507-515, 2000.
Remington's Pharmaceutical Sciences, 15^{th} ed., pages 1035-1038 and 1570-1580, Mack Publishing Company, Easton, Pa., 1980.
Restifo et al., J. Exp. Med., 177:265-272, 1993.
Riddell and Greenberg, Annu. Rev. Immunol., 13:545-586, 1995.
Riddell and Greenberg, Cancer Treat Res., 76:337-369, 1995.
Riddell and Greenberg, Curr. Top Microbiol. Immunol., 189:9-34, 1994.
Riddell et al., Science, 257:238-241, 1992.
Ridgeway, In: Vectors: A survey of molecular cloning vectors and their uses, Rodriguez and Denhardt (Eds.), Stoneham:Butterworth, 467-492, 1988.
Rippe et al., Mol. Cell Biol., 10:689-695, 1990.
Rosenberg and White, J. Immunother. Emphasis. Tumor Immunol., 19:81-84, 1996.
Rosenberg et al., Ann. Surg. 210(4):474-548, 1989.
Rosenberg et al., Nature Med., 4:321-327, 1998.
Rosenberg et al., N. Engl. J Med., 319:1676, 1988.
Salter and Cresswell, Embo. J., 5:943-949, 1986.
Santinietal., J. Exp. Med., 191:1777-1788, 2000.
Savage et al., Immunity, 10:485-492, 1999.
Savige et al., Am. J Clin. Pathol., 111:507-513, 1999.
Scheibenbogen et al., Blood, 100:2132-2137, 2002.
Scheibenbogen et al., Int. J. Cancer, 71:932-936, 1997.
Serrano et al., Nature, 366:704-707, 1993.
Serrano et al., Science, 267(5195):249-252, 1995.
Sette et al., J. Immunol., 153:5586-5592, 1994.
Shlomchik et al., Science, 285:412-415, 1999.
Sibelius et al., J. Exp. Med., 187:497-503, 1998.
Snapper et al., Proc. Natl. Acad. Sci. USA, 85(18):6987-6991, 1988.
Stark, G. R., Kerr, I. M., Williams, B. R., Silverman, R. H. & Schreiber, R. D. How cells respond to interferons. Annu Rev Biochem 67, 227-64 (1998).
Sturrock et al., J. Biol. Chem., 267:21193, 1992.
Takada et al., J. Clin. Microbiol., 33(3):658-660, 1995.
Tanaka et al., Cancer Res., 57:4465-4468, 1997.
Tanaka et al., Genes Cells, 3:29-37, 1998.
Temin, In: Gene Transfer, Kucherlapati (Ed.), NY, Plenum Press, 149-188, 1986.
Teshima et al., Nat. Med., 8:575-581, 2002.
Toes et al., Int. J. Cancer, 66:686-691, 1996.
Toes et al., J. Immunol., 156:3911-3918, 1996.
Toes et al., Proc. Natl. Acad. Sci. USA, 93:7855-7860, 1996.
Tratschin et al., Mol. Cell. Biol., 4:2072-2081, 1984.
Tsujimoto and Croce, Proc. Natl. Acad. Sci. USA, 83(14):5214-5218, 1986.
Tsujimoto et al., Science, 228(4706):1440-1443, 1985.
Tsukada et al., J. Exp. Clin. CancerRes., 16:171-176, 1997.
van der Bruggen et al., Eur. J. Immunol., 124:3038-3043, 1994.
van der Harst et al., Blood, 83:1060-1066, 1994.
van Lochem et al., Bone Marrow Transplant., 10:181-183, 1992.
van Rhee et al., Blood, 83, 1994.
Verdijk et al., J. Immunol., 163:57-61, 1999.
Wada et al., Nucleic Acids Res. 18:2367-2411, 1990.
Wang et al., Biol. Blood Marrow Transplant., 6:118, 2000.
Weinberger et al., Science, 228:740-742, 1985.
Willians et al., J. Immunol., 152:4722-4732, 1994.
Wolfet al., Comput. Appl. Biosci., 4(1):187-191, 1988.
Wong et al., Gene, 10:87-94, 1980.
Yamamoto et al., Jpn. J. Cancer Res., 79:866-873, 1988.
Yee et al., J. Immunol., 162:2227-2234, 1999.
Yin et al., J. Biol. Resp. Modif., 8:190-205, 1989.
Zarour et al., J. Invest. Dermatol., 107:63-67, 1996.
Zeh et al., J. Immunol., 162:989-994, 1999.
Zufferey et al., Nat. Biotechnol., 15(9):871-875, 1997.

What is claimed is:

1. A method for treating an autoimmune disease in a patient comprising administering to said patient a therapeutically effective amount of a vaccine comprising a HLA-A2-restricted peptide, wherein the HLA-A2-restricted peptide is a proteinase-3 peptide, a neutrophil elastase peptide, a myeloperoxidase peptide, a cyclin E1 peptide, cyclin D, or a cyclin E2 peptide.

2. The method of claim 1, wherein the cyclin D comprises the sequence LLGATCMFV (SEQ ID NO:61) or a fragment thereof, or wherein the cyclin E1 peptide comprises the sequence ILLDWLMEV (SEQ ID NO:62), or wherein the cyclin E2 peptide comprises the sequence ILLDWLLEV (SEQ ID NO:63) or a fragment thereof, or wherein the myleoperoxidase peptide may comprise the sequence of MY2 (SEQ ID NO:32) or MY4 (SEQ ID NO:30) or a fragment thereof.

3. The method of claim 1, wherein the vaccine further comprises a disease-associated HLA-A3 restricted peptide.

4. The method of claim 1, wherein the vaccine further comprises a disease-associated HLA-A11 restricted peptide.

5. The method of claim 1, wherein the vaccine further comprises a disease-associated HLA-B7 restricted peptide.

6. The method of claim 1, wherein the vaccine further comprises a disease-associated HLA-B27 restricted peptide.

7. The method of claim 1, wherein the vaccine further comprises a disease-associated HLA-B35 restricted peptide.

8. The method of claim 1, wherein the proteinase-3 peptide is selected from the group consisting of VLQELNVTV (SEQ ID NO:1), RFLPDFFTRV (SEQ ID NO:3), VLQELNVTVV (SEQ ID NO:4), NLSASVTSV (SEQ ID NO:5), IIQGIDSFV (SEQ ID NO:6), VLLALLLISGA (SEQ ID NO:7), QLPQQDQPV
(SEQ ID NO:10) and FLNNYDAENKL (SEQ ID NO:1 1) or a fragment thereof.

9. The method of claim 1, wherein the proteinase-3 peptide is a modified peptide selected from the group consisting of VLQELWTV (SEQ ID NO:26), VLQELNVKV (SEQ ID NO:27), VLQELWKV (SEQ ID NO:28) and VMQELWTV (SEQ ID NO:29) or a fragment thereof.

10. The method of claim 1, wherein the autoimmune disease is vasculitis, Wegener's granulomatosis, Addison's disease, alopecia, ankylosing spondylitis, antiphospholipid syndrome, Behcet's disease, celiac disease, chronic fatigue syndrome, Crohn's disease, ulcerative colitis, type I diabetes, fibromyalgia, autoimmune gastritis, Goodpasture syndrome, Graves' disease, idiopathic thrombocytopenic purpura (ITP), lupus, Meniere's multiple sclerosis, myasthenia gravis, pemphigus vulgaris, primary biliary cirrhosis, psoriasis, rheumatoid arthritis, rheumatic fever, sarcoidosis, scleroderma, vitiligo, vasculitis, small vessel vasculitis, hepatitis, primary biliary cirrhosis, rheumatoid arthritis, Chrohn's disease, ulcerative colitis, sarcoidosis, scleroderma, graft versus host disease (acute and chronic), aplastic anemia, or cyclic neutropenia.

11. A method of treating or preventing an infectious disease in a patient comprising administering to said patient a therapeutically effective amount of a vaccine comprising a HLA-A2-restricted peptide, wherein the HLA-A2-restricted peptide is a proteinase-3 peptide, a neutrophil elastase peptide, a myeloperoxidase peptide, a cyclin E1 peptide, cyclin D, or a cyclin E2 peptide.

12. The method of claim 11, wherein the cyclin D comprises the sequence LLGATCMFV (SEQ ID NO:61) or a fragment thereof, or wherein the cyclin E1 peptide comprises the sequence ILLDWLMEV (SEQ ID NO:62), or wherein the cyclin E2 peptide comprises the sequence ILLDWLLEV (SEQ ID NO:63) or fragment thereof, or wherein the myleoperoxidase peptide is MY2 (SEQ ID NO:32) or MY4 (SEQ ID NO:30) or a fragment thereof.

13. The method of claim 11, further comprising a disease-associated HLA-A3 restricted peptide.

14. The method of claim 11, further comprising a disease-associated HLA-A11 restricted peptide.

15. The method of claim 11, further comprising a disease-associated HLA-B7 restricted peptide.

16. The method of claim 11, further comprising a disease-associated HLA-B27 restricted peptide.

17. The method of claim 11, further comprising a disease-associated HLA-B35 restricted peptide.

18. The method of claim 11, wherein the proteinase-3 peptide is selected from the group consisting of VLQELNVTV (SEQ ID NO:1), RFLPDFFTRV (SEQ ID NO:3), VLQELNVTVV (SEQ ID NO:4), NLSASVTSV (SEQ ID NO:5), IIQGIDSFV (SEQ ID NO:6), VLLALLLISGA (SEQ ID NO:7), QLPQQDQPV
(SEQ ID NO:1O) and FLNNYDAENKL (SEQ ID NO:1 1) or a fragment thereof.

19. The method of claim 11, wherein the proteinase-3 peptide is a modified peptide selected from the group consisting of VLQELWTV (SEQ ID NO:26), VLQELNVKV (SEQ ID NO:27), VLQELWKV (SEQ ID NO:28) and VMQELWTV (SEQ ID NO:29) or fragment thereof.

20. The method of claim 11, wherein the infectious disease is a viral hepatitis.

21. The method of claim 20, wherein the viral hepatitis is hepatitis B or hepatitis C.

22. A method of generating HLA-restricted peptide specific cytotoxic T-lymphocytes (CTLs) comprising:
(a) obtaining a target peptide pulsed onto antigen-presenting cells to expand the CTL ex vivo;
(b) concentrating high affinity HLA-restricted peptide specific CTLs; and
(c) expanding the concentrated HLA-restricted peptide specific CTLs.

23. The method of claim 22, further comprising purifying the concentrated HLA-restricted peptide specific CTLs.

24. The method of claim 22, wherein step (b) may occur prior to, after, or both before and after purifying the concentrated HLA-restricted peptide-specific CTLs.

25. The method of claim 23, wherein purifying comprises high-speed flow cytometry.

26. The method of claim 22, wherein the target peptide depends upon the donor and the peptide epitope.

27. The method of claim 22, wherein concentrating high affinity HLA-restricted peptide specific CTLs comprises using microbeads.

28. The method of claim 22, wherein concentrating high affinity HLA-restricted peptide specific CTLs comprises using high speed sorting.

29. The method of claim 22, further comprising concentrating low affinity HLA-restricted peptide specific CTLs.

30. The method of claim 29, comprising expanding the concentrated low affinity HLA-restricted peptide specific CTLs.

31. The method of claim 22, further comprising adoptive T cell immunotherapy.

32. The method of claim 22, further comprising expansion of adoptively transferred peptide-specific T lymphocytes.

33. The method of claim 32, further comprising vaccinating the recipient with the specific peptide combined with an adjuvant after the transfer of the peptide-specific T lymphocytes.

34. A method for treating or preventing an autoimmune or infectious disease in a patient comprising:
(a) contacting CTLs of said patient with a HLA-A2-restricted peptide, wherein the HLA-A2-restricted peptide is a proteinase 3 peptide, a neutrophil elastase peptide, a myeloperoxidase peptide, a cyclin E1 peptide, cyclin D, or a cyclin E2 peptide; and
(b) administering a therapeutically effective amount of said CTLs to the patient.

35. The method of claim 34, further comprising expanding said CTL's by ex vivo or in vivo methods prior to administration.

36. The method of claim 34, wherein contacting comprises providing an antigen presenting cell containing said peptide.

37. The method of claim 34, further comprising providing CTLs transfected with a T cell receptor specific for the peptide/HLA-A2 ligand.

38. The method of claim 34, wherein the autoimmune disease is vasculitis, Wegner's granulomatoses Addison's disease, alopecia, ankylosing spondylitis, antiphospholipid syndrome, Behcet's disease, celiac disease, chronic fatigue syndrome, Crohn's disease, ulcerative colitis, type I diabetes, fibromyalgia, autoimmune gastritis, Goodpasture syndrome, Graves' disease, idiopathic thrombocytopenic purpura (ITP), lupus, Meniere's multiple sclerosis, myasthenia gravis, pemphigus vulgaris, primary biliary cirrhosis, psoriasis, rheumatoid arthritis, rheumatic fever, sarcoidosis, scleroderma, vitiligo, vasculitis, small vessel vasculitis, hepatitis, primary biliary cirrhosis, rheumatoid arthritis, Chrohn's disease, ulcerative colitis, sarcoidosis, scleroderma, graft versus host disease (acute and chronic), aplastic anemia, myelodysplastic syndrome, cyclic neutropenia, T-LGL (T-large granular lymphocytic) leukemia.

39. The method of claim 34, wherein the infectious disease is a viral hepatitis.

40. The method of claim 39, wherein the viral hepatitis is hepatitis B or hepatitis C.

41. The method of claim 34, wherein the therapeutically effective amount of CTL cells required to provide therapeutic benefit is from about 0.1×10⁵ to about 5×10⁷ cells per kilogram weight of the subject.

42. A method for treating or preventing an autoimmune disease in a patient comprising administering to said patient a therapeutically effective amount of a vaccine comprising an expression construct encoding a tumor associated HLA-A2-restricted peptide, wherein the HLA-A2-restricted peptide is a proteinase-3 peptide, a neutrophil elastase peptide, a cyclin E1 peptide, cyclin D, or a cyclin E2 peptide.

43. The method of claim 42, wherein said expression construct is a non-viral expression construct.

44. The method of claim 42, wherein said expression construct is a viral expression construct.

45. A method for preventing an autoimmune disease in a patient comprising (a) identifying a patient at risk of developing an autoimmune disease, and (b) administering to said patient a therapeutically effective amount of a vaccine comprising a HLA-A2-restricted peptide, wherein the HLA-A2-restricted peptide is a proteinase-3 peptide, a neutrophil elastase peptide, a myeloperoxidase peptide, a cyclin E1 peptide, cyclin D, or a cyclin E2 peptide.

46. A method of generating HLA-restricted peptide specific cytotoxic T-lymphocytes (CTLs) comprising:
(a) obtaining a target peptide pulsed onto antigen-presenting cells to expand the CTL ex vivo;
(b) concentrating low affinity HLA-restricted peptide specific CTLs; and
(c) expanding the concentrated HLA-restricted peptide specific CTLs.

47. The method of claim 46, further comprising purifying the concentrated HLA-restricted peptide specific CTLs.

48. The method of claim 46, wherein step (b) may occur prior to, after, or both before and after purifying the concentrated HLA-restricted peptide-specific CTLs.

49. The method of claim 48, wherein purifying comprises high-speed flow cytometry.

50. The method of claim 46, wherein the target peptide depends upon the donor and the peptide epitope.

51. The method of claim 46, wherein concentrating low affinity HLA-restricted peptide specific CTLs comprises using microbeads.

52. The method of claim 46, wherein concentrating low affinity HLA-restricted peptide specific CTLs comprises using high speed sorting.

53. The method of claim 46, further comprising concentrating high affinity HLA-restricted peptide specific CTLs.

54. The method of claim 53, comprising expanding the concentrated high affinity HLA-restricted peptide specific CTLs.

55. The method of claim 46, further comprising adoptive T cell immunotherapy.

56. The method of claim 46, further comprising expansion of adoptively transferred peptide-specific T lymphocytes.

57. The method of claim 56, further comprising vaccinating the recipient with the specific peptide combined with an adjuvant after the transfer of the peptide-specific T lymphocytes.

58. A method of generating HLA-restricted peptide specific regulatory T-cells comprising:
(a) obtaining a target peptide pulsed onto antigen-presenting cells to expand the T-cells ex vivo;
(b) concentrating HLA-restricted peptide specific regulatory T-cells; and
(c) expanding the concentrated HLA-restricted peptide specific regulatory T-cells.


SEQUENCE LISTING (63 sequences)

SEQ ID NO: 1 - protein, 9 aa, Artificial Sequence
  Description of Artificial Sequence  Synthetic Peptide
VLQELNVTV

SEQ ID NO: 2 - protein, 9 aa, Artificial Sequence
  Description of Artificial Sequence  Synthetic Peptide
NLVPMVATV

SEQ ID NO: 3 - protein, 10 aa, Artificial Sequence
  Description of Artificial Sequence  Synthetic Peptide
RLFPDFFTRV

SEQ ID NO: 4 - protein, 10 aa, Artificial Sequence
  Description of Artificial Sequence  Synthetic Peptide
VLQELNVTVV

SEQ ID NO: 5 - protein, 9 aa, Artificial Sequence
  Description of Artificial Sequence  Synthetic Peptide
NLSASVTSV

SEQ ID NO: 6 - protein, 9 aa, Artificial Sequence
  Description of Artificial Sequence  Synthetic Peptide
IIQGIDSFV

SEQ ID NO: 7 - protein, 11 aa, Artificial Sequence
  Description of Artificial Sequence  Synthetic Peptide
VLLALLLISGA

SEQ ID NO: 8 - protein, 10 aa, Artificial Sequence
  Description of Artificial Sequence  Synthetic Peptide
KLNDILLIQL

SEQ ID NO: 9 - protein, 10 aa, Artificial Sequence
  Description of Artificial Sequence  Synthetic Peptide
KLNDVLLIQL

SEQ ID NO: 10 - protein, 9 aa, Artificial Sequence
  Description of Artificial Sequence  Synthetic Peptide
QLPQQDQPV

SEQ ID NO: 11 - protein, 11 aa, Artificial Sequence
  Description of Artificial Sequence  Synthetic Peptide
FLNNYDAENKL

SEQ ID NO: 12 - DNA, 20 bp, Artificial Sequence
  Description of Artificial Sequence  Synthetic Primer
ctggaccccaccatggctca

SEQ ID NO: 13 - DNA, 20 bp, Artificial Sequence
  Description of Artificial Sequence  Synthetic Primer
cgccacagtgttcggggaag

SEQ ID NO: 14 - DNA, 18 bp, Artificial Sequence
  Description of Artificial Sequence  Synthetic Primer
tgtaaaacgacggccagt

SEQ ID NO: 15 - DNA, 18 bp, Artificial Sequence
  Description of Artificial Sequence  Synthetic Primer
caggaaacagctatgacc

SEQ ID NO: 16 - DNA, 20 bp, Artificial Sequence
  Description of Artificial Sequence  Synthetic Primer
actcaactccgtctggcatt

SEQ ID NO: 17 - DNA, 20 bp, Artificial Sequence
  Description of Artificial Sequence  Synthetic Primer
tgatgacctcgtggtggata

SEQ ID NO: 18 - DNA, 20 bp, Artificial Sequence
  Description of Artificial Sequence  Synthetic Primer
gctccctgacgcctggactc

SEQ ID NO: 19 - DNA, 20 bp, Artificial Sequence
  Description of Artificial Sequence  Synthetic Primer
acggggcttagctgggtcct

SEQ ID NO: 20 - DNA, 20 bp, Artificial Sequence
  Description of Artificial Sequence  Synthetic Primer
ccggggaggacccagctaag

SEQ ID NO: 21 - DNA, 20 bp, Artificial Sequence
  Description of Artificial Sequence  Synthetic Primer
ggtcgtggcccggtatacag

SEQ ID NO: 22 - DNA, 20 bp, Artificial Sequence
  Description of Artificial Sequence  Synthetic Primer
tttgaggtggtgggtgtggt

SEQ ID NO: 23 - DNA, 20 bp, Artificial Sequence
  Description of Artificial Sequence  Synthetic Primer
aggcacagcatgaagccaca

SEQ ID NO: 24 - DNA, 20 bp, Artificial Sequence
  Description of Artificial Sequence  Synthetic Primer
tcaggtggccctgatgggtg

SEQ ID NO: 25 - DNA, 20 bp, Artificial Sequence
  Description of Artificial Sequence  Synthetic Primer
tcgagggtttggagccaggc

SEQ ID NO: 26 - protein, 8 aa, Artificial Sequence
  Description of Artificial Sequence  Synthetic Peptide
VLQELWTV

SEQ ID NO: 27 - protein, 9 aa, Artificial Sequence
  Description of Artificial Sequence  Synthetic Peptide
VLQELNVKV

SEQ ID NO: 28 - protein, 8 aa, Artificial Sequence
  Description of Artificial Sequence  Synthetic Peptide
VLQELWKV

SEQ ID NO: 29 - protein, 8 aa, Artificial Sequence
  Description of Artificial Sequence  Synthetic Peptide
VMQELWTV

SEQ ID NO: 30 - protein, 9 aa, Artificial Sequence
  Description of Artificial Sequence  Synthetic Peptide
RLFEQVMRI

SEQ ID NO: 31 - protein, 9 aa, Artificial Sequence
  Description of Artificial Sequence  Synthetic Peptide
SLWRRPFNV

SEQ ID NO: 32 - protein, 9 aa, Artificial Sequence
  Description of Artificial Sequence  Synthetic Peptide
KLLLALAGL

SEQ ID NO: 33 - protein, 9 aa, Artificial Sequence
  Description of Artificial Sequence  Synthetic Peptide
LLSYFKQPV

SEQ ID NO: 34 - protein, 9 aa, Artificial Sequence
  Description of Artificial Sequence  Synthetic Peptide
LIQPFMFRL

SEQ ID NO: 35 - protein, 9 aa, Artificial Sequence
  Description of Artificial Sequence  Synthetic Peptide
RVFFASWRV

SEQ ID NO: 36 - protein, 9 aa, Artificial Sequence
  Description of Artificial Sequence  Synthetic Peptide
VLGEVDTSL

SEQ ID NO: 37 - protein, 9 aa, Artificial Sequence
  Description of Artificial Sequence  Synthetic Peptide
LLLREHNRL

SEQ ID NO: 38 - protein, 9 aa, Artificial Sequence
  Description of Artificial Sequence  Synthetic Peptide
VLTPAQLNV

SEQ ID NO: 39 - protein, 9 aa, Artificial Sequence
  Description of Artificial Sequence  Synthetic Peptide
YLHVALDLL

SEQ ID NO: 40 - DNA, 23 bp, Artificial Sequence
  Description of Artificial Sequence  Synthetic Primer
catctgcttcggagactcaggtg

SEQ ID NO: 41 - DNA, 18 bp, Artificial Sequence
  Description of Artificial Sequence  Synthetic Primer
tcagggaaaaggcgggtg

SEQ ID NO: 42 - protein, 9 aa, Artificial Sequence
  Description of Artificial Sequence  Synthetic Peptide
VLGPTAMRK

SEQ ID NO: 43 - protein, 9 aa, Artificial Sequence
  Description of Artificial Sequence  Synthetic Peptide
GLPGYNAWR

SEQ ID NO: 44 - protein, 9 aa, Artificial Sequence
  Description of Artificial Sequence  Synthetic Peptide
TLIQPFMFR

SEQ ID NO: 45 - protein, 9 aa, Artificial Sequence
  Description of Artificial Sequence  Synthetic Peptide
VLSSMEEAK

SEQ ID NO: 46 - protein, 9 aa, Artificial Sequence
  Description of Artificial Sequence  Synthetic Peptide
FMFRLDNRY

SEQ ID NO: 47 - protein, 9 aa, Artificial Sequence
  Description of Artificial Sequence  Synthetic Peptide
AMRKYLPTY

SEQ ID NO: 48 - protein, 9 aa, Artificial Sequence
  Description of Artificial Sequence  Synthetic Peptide
AMVQIITYR

SEQ ID NO: 49 - protein, 9 aa, Artificial Sequence
  Description of Artificial Sequence  Synthetic Peptide
CIIGTQFRK

SEQ ID NO: 50 - protein, 9 aa, Artificial Sequence
  Description of Artificial Sequence  Synthetic Peptide
GLLAVNQRF

SEQ ID NO: 51 - protein, 9 aa, Artificial Sequence
  Description of Artificial Sequence  Synthetic Peptide
NPRWDGERL

SEQ ID NO: 52 - protein, 9 aa, Artificial Sequence
  Description of Artificial Sequence  Synthetic Peptide
IVRFPTDQL

SEQ ID NO: 53 - protein, 9 aa, Artificial Sequence
  Description of Artificial Sequence  Synthetic Peptide
GPTAMRKYL

SEQ ID NO: 54 - protein, 9 aa, Artificial Sequence
  Description of Artificial Sequence  Synthetic Peptide
RPFNVTDVL

SEQ ID NO: 55 - protein, 9 aa, Artificial Sequence
  Description of Artificial Sequence  Synthetic Peptide
QVMRIGLDL

SEQ ID NO: 56 - protein, 9 aa, Artificial Sequence
  Description of Artificial Sequence  Synthetic Peptide
NPRVPLSRV

SEQ ID NO: 57 - protein, 9 aa, Artificial Sequence
  Description of Artificial Sequence  Synthetic Peptide
MQRSRDHGL

SEQ ID NO: 58 - protein, 9 aa, Artificial Sequence
  Description of Artificial Sequence  Synthetic Peptide
SNRAFVRWL

SEQ ID NO: 59 - protein, 9 aa, Artificial Sequence
  Description of Artificial Sequence  Synthetic Peptide
TIRNQINAL

SEQ ID NO: 60 - protein, 9 aa, Artificial Sequence
  Description of Artificial Sequence  Synthetic Peptide
NLRNMSNQL

SEQ ID NO: 61 - protein, 9 aa, Artificial Sequence
  Description of Artificial Sequence  Synthetic Peptide
LLGATCMFV

SEQ ID NO: 62 - protein, 9 aa, Artificial Sequence
  Description of Artificial Sequence  Synthetic Peptide
ILLDWLMEV

SEQ ID NO: 63 - protein, 9 aa, Artificial Sequence
  Description of Artificial Sequence  Synthetic Peptide
ILLDWLLEV